(19) United States
(12) Patent Application Publication
(10) Pub. No.: US 2007/0118398 A1
(43) Pub. Date: May 24, 2007

(54) System and method for estimating life expectancy and providing customized advice for improving life expectancy

(75) Inventor: Thomas T. Perls, Weston, MA (US)

Correspondence Address: ROBERT PLOTKIN, PC, 91 MAIN STREET, SUITE 204, CONCORD, MA, 01742-2527, US

(73) Assignee: Flicker Technologies, LLC

(21) Appl. No.: 11/283,469

(22) Filed: Nov. 18, 2005

(51) Int. Cl. G06Q 10/00; A61B 5/00

(52) U.S. Cl. 705/2; 600/300

(57) ABSTRACT

A system is disclosed for estimating a person's life expectancy and for providing customized advice for improving the person's life expectancy. The system may, for example, be accessible to the person through a website, which may provide the person with a questionnaire containing questions to which the person responds. The system calculates and provides the person with an estimate of the person's life expectancy based on the person's responses to the questionnaire. The system may provide the person with the estimated life expectancy free of charge, while requiring the person to pay a fee for health-related feedback that is tailored to the person and intended to enable the person to increase his or her life expectancy. The system may be customized for entities such as insurance companies and corporate human resources departments so that their clients and employees may receive feedback that is specific to each user.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

1. Field of the Invention

The present invention relates to techniques for evaluating a person's health and to providing health advice to the person based on the evaluation.

2. Related Art

Life expectancy in the United States currently is approximately 77 years. Based upon findings from the Seventh Day Adventist Study based at Loma Linda University in Loma Linda, Calif., the average American should be able to live more than 10 years longer than he or she does now, and in much better health. With health habits including a vegetarian diet, frequent exercise, not smoking, not drinking alcohol (though a small amount of wine every day is likely beneficial) and the ability to handle stress well, Seventh Day Adventists have an average life expectancy of 88 years. G. E. Fraser and D. J. Shavlik, Ten years of life: Is it a matter of choice?, Arch Intern Med 161 (2001), no. 13, 1645-1652.

Furthermore, data from the Adventist study and the New England Centenarian Study (led by Dr. Thomas Perls) and other studies support the conclusion that living to this older age (88 and older), rather than to the current life expectancy, entails better health and compresses the time people are sick into a shorter period of time near the ends of their lives. R. Hitt, Y. Young-Xu, M. Silver and T. Perls, Centenarians: The older you get, the healthier you have been, Lancet 354 (1999), no. 9179, 652.

Instead of the old inaccurate adage of “the older you get, the sicker you get,” it is much more likely that “the older you get, the healthier you've been.” Thus living to significantly older age can be a huge advantage both in quantity and quality of years lived. Furthermore, health care costs are much lower when people are able to live to their true life expectancy potential (which again is, for most people, the mid to late eighties). T. Perls and E. Wood, Acute care costs of the oldest old: They cost less, their care intensity is less and they go to non-teaching hospitals, Archives of Internal Medicine 156 (1996), 754-760.

Unfortunately, a relatively small portion of the health care dollar is dedicated to educating the public about the dramatic impact the improvement of a relatively few, but very important, health related behaviors can have on the life expectancy and quality of life of individuals and the public as a whole. Although a great deal of health-related information has become available on the Internet, the majority of this information focuses on the diagnosis and treatment of specific diseases. Other websites, in a display that is closer to hucksterism and quackery than to valid health information, focus on selling products that purportedly cure diseases, syndromes, or even aging that have little or no valid scientific bases.

In addition to such websites, various patents and published patent applications are related to techniques for assessing human health and for providing health-related information. Examples of such patents and patent applications include the following. U.S. Pat. No. 5,937,387 to Summerell et al. discloses a system and method for developing and selecting a customized wellness plan by determining a user's “physiological age.” According to the patent, “physiological age is equal to the chronologic age at which the user's survival probability rate equals the average survival probability rate of an individual of the same gender. Stated in another way, a user's physiological age is equal to the calendar age of an average person of the same gender with a comparable risk stratification level. Thus, a user's physiological age is a metric; that is, it is a meaningful number used to measure wellness.” The various techniques described in the patent, such as those for choosing wellness options and for determining the potential effect that implementing the options could have on the user, are all based on the concept of “physiological age.”

U.S. Pat. No. 6,206,829 B1 to Iliff discloses techniques for providing differential diagnoses and subsequent medical advice via the telephone using voice recognition software and the Internet based upon a user's responses to specific questions. The system is also meant to replace the need to see a physician in order to make initial diagnoses and treatment plans.

U.S. Pat. No. 6,334,192 B1 to Karpf discloses a computerized system for assessing risk for certain diseases based upon the user's responses to specific questions. The example of assessing for risk of myocardial infarction is used in the patent. The invention also uses a number of complex statistical tools including logistic regression to provide the risk assessment.

U.S. Pat. App. Pub. No. US 2003/0074226 A1 to Rostron et al. discloses techniques for using standardized actuarial data tables in a computerized system to determine a client's life expectancy ultimately followed by a calculation of premiums. The purpose of the disclosed calculations is to assess insurability of these insurance products.

U.S. Pat. No. 6,584,446 B1 to Buchanan et al. discloses techniques for using a set of risk algorithms to assess married couples for insurability for joint life and long term life insurance policies. The purpose of the life expectancy calculations that are performed is to assess insurability of these insurance products.

U.S. Pat. App. Pub. No. 2003/0101075 A1 to Hideyuki et al. discloses techniques for using health screening data and a medical payment prediction knowledge data base (medical fee bill data records) to predict the number of healthy years that a client still has ahead of him or her. This calculation, which takes into account age, gender, smoking history, alcohol use, exercise and the absence or presence of obesity, hypertension, hyperlipidemia, hyperglycemia, and hyperuricemia, is in turn used to support medical insurer management of the client such as recommendations for frequency of subsequent screening and treatment and/or prevention strategies.

U.S. Pat. App. Pub. No. 2003/0208385 A1 to Zander et al. discloses techniques for assisting underwriters in evaluating life insurance applications using ratings chosen by the insurer based upon their review of the client's insurance application. The calculated life expectancy is provided as well as the impact of various determinants (e.g., smoking) upon the client's life expectancy.

These and other prior art techniques are limited in their ability to evaluate a person's health and to provide health advice to the person based on the evaluation. What is needed, therefore, are improved techniques for providing such services.

### SUMMARY

A system is disclosed for estimating a person's life expectancy and for providing customized advice for improving the person's life expectancy. The system may, for example, be accessible to the person through a website. The website may provide the person with a questionnaire containing questions to which the person responds. The selection and sequence of questions posed to the person may vary depending upon factors such as the person's expressed preferences and answers provided by the person to previous questions. Once the person completes the questionnaire, the system identifies an estimate of the person's life expectancy and provides the person with the estimated life expectancy.

The system allows the person, such as by using hyperlinks or referrals to other websites, to obtain more detailed information or access to other resources that are pertinent to specific health behavior or medical issues that apply to or are specific to the person based upon the person's answers to the life expectancy questionnaire. The person may be provided with the opportunity to retake the questionnaire and answer the questions differently in order to see the impact of a certain behavior or medical issue upon his or her calculated life expectancy. The person may be provided with the option of storing his or her data on the system, changing previous answers to the questionnaire at any time, and keeping track of how such changes impact upon the estimated life expectancy.

In one embodiment, the system provides the person with the estimated life expectancy free of charge, while requiring the person to pay a fee for health-related feedback that is tailored to the person and intended to enable the person to increase his or her life expectancy. The system may provide the opportunity for advertisers or sponsors to advertise their products and/or services using the system (such as through a website) for a fee. The system may be tailor-made for insurance companies, human resources departments of businesses, and other entities so that their clients and employees may receive feedback that includes information, resources and links uniquely available to the clients and/or employees.

For example, in one embodiment of the present invention, a computer-implemented method is provided that includes: (A) providing health-related questions to a user; (B) receiving answers to the questions from the user; (C) identifying an estimate of a life expectancy of the user based on the received answers without charging the user a fee; and (D) providing advice to the user for increasing the life expectancy based on the received answers in exchange for a fee paid by the user.

In another embodiment of the present invention, a computer-implemented method is provided that includes: (A) providing health-related questions to a user; (B) receiving answers to the questions from the user; (C) identifying an estimate of a life expectancy of the user based on the received answers; (D) identifying an age of the user; (E) identifying a gender of the user; and (F) based on the received answers, the identified age, and the identified gender, providing advice to the user for increasing the life expectancy by following a plan of prevention to be carried out with a health-care provider of the user.

In yet another embodiment of the present invention, a method is provided that includes: (A) charging an organization a fee to obtain access to a computer-implemented life expectancy system; (B) providing the organization with access to the life expectancy system, wherein the life expectancy system: (1) provides health-related questions to a member of the organization; (2) receives answers to the questions from the member; (3) identifies an estimate of a life expectancy of the member based on the received answers; and (4) provides advice to the member for increasing the life expectancy based on the received answers; wherein (B)(1)-(B)(4) are performed without charging an additional fee to the member.

In yet a further embodiment of the present invention, a method is provided that includes: (A) charging an entity a fee to obtain access to a computer-implemented life expectancy system; (B) providing the entity with access to the life expectancy system, wherein the life expectancy system: (1) provides health-related questions to a person selected by the entity; (2) receives answers to the questions from the person; (3) identifies an estimate of a life expectancy of the person based on the received answers; and (4) recommends advice to provide to the person for increasing the life expectancy based on the received answers.

Other features and advantages of various aspects and embodiments of the present invention will become apparent from the following description and from the claims.

### BRIEF DESCRIPTION OF THE DRAWINGS

FIG. 1 is a data flow diagram of a life expectancy system according to one embodiment of the present invention; and

FIG. 2 is a flow chart of a method performed by the system of FIG. 1 according to one embodiment of the present invention.

### DETAILED DESCRIPTION

Embodiments of the present invention relate generally to techniques for: (1) estimating the life expectancy of a person based on factors such as the person's health habits, family history of illnesses and longevity, medical history, and medical characteristics, as indicated by the person in responses to a questionnaire; and (2) providing the person with automated feedback based upon the person's answers to the questionnaire. One example of a questionnaire that is suitable for use with various embodiments of the present invention may be found in the book Living to 100: Lessons in Living to Your Maximum Potential at any Age, written by T. Perls, J. Lauerman, and M. Silver (New York: Basic Books, 1999), the copyright in which is owned by T. Perls, and which is hereby incorporated by reference herein. Other examples of questionnaires suitable for use with various embodiments of the present invention may be found on the website www.livingto100.com.

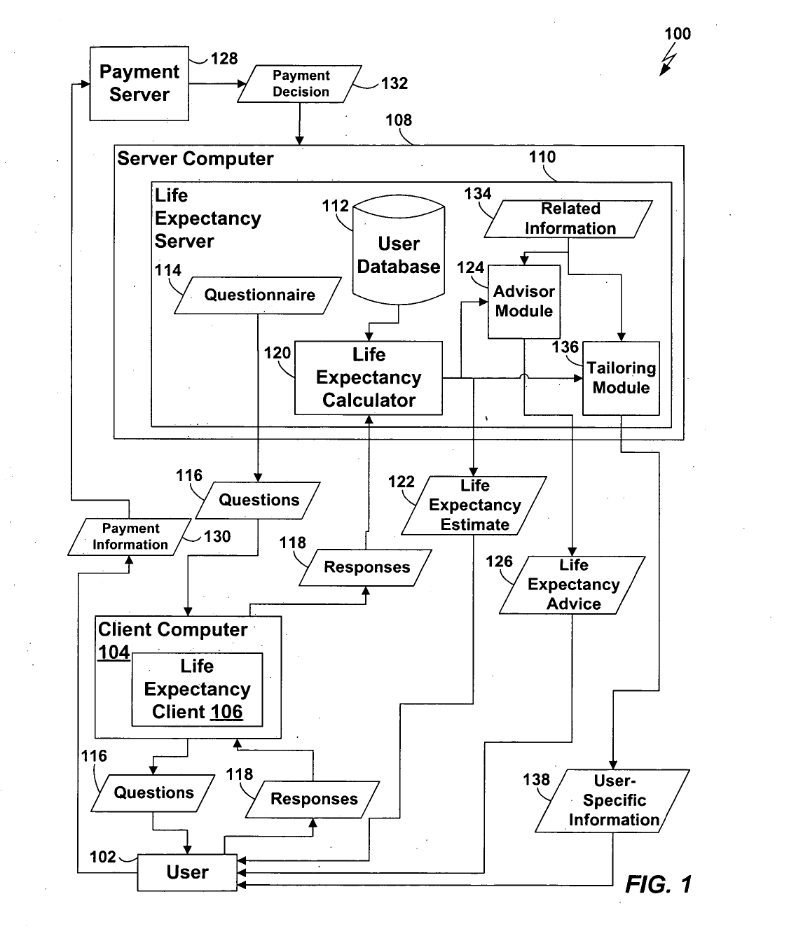

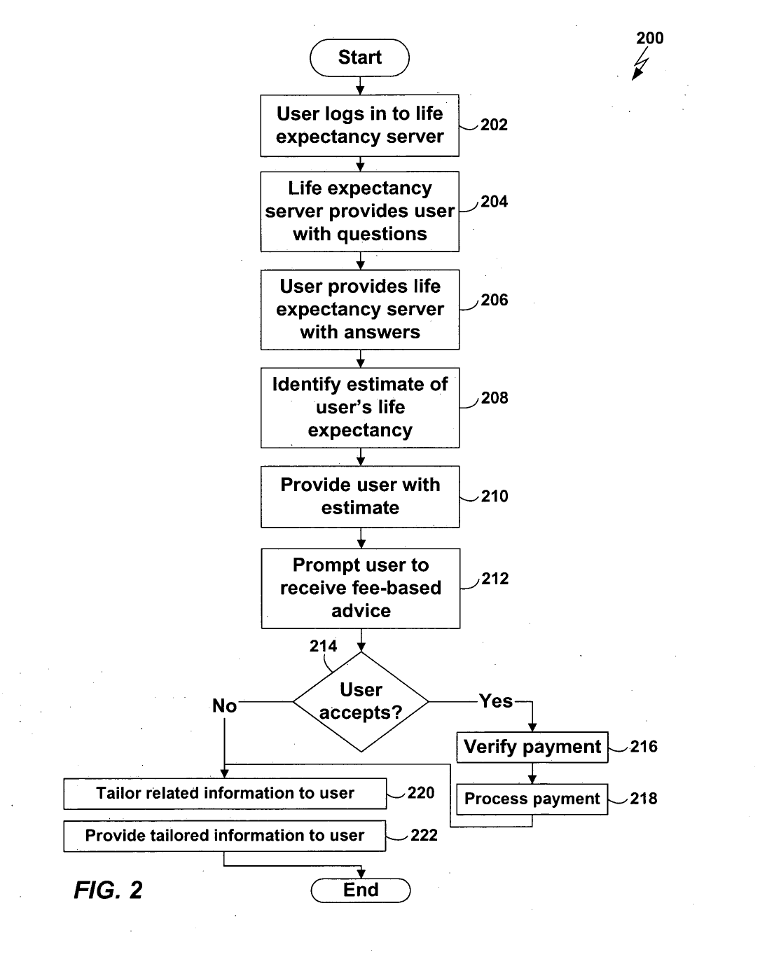

For example, referring to FIG. 1, a data flow diagram of a system 100 according to one embodiment of the present invention is shown. For ease of illustration and explanation, elements in FIG. 1 are shown as black boxes. Those having ordinary skill in the art, however, will understand how to implement elements of FIG. 1 using appropriate technology in accordance with the description provided herein. Referring to FIG. 2, a flowchart is shown of a method 200 that is performed by the system 100 of FIG. 1 according to one embodiment of the present invention.

The system 100 is shown as having a single user 102, although in practice any number of users may use the system 100. The system 100 includes a client computer 104 with which the user 102 interacts. The client computer 104 may, for example, be any conventional computer, such as a desktop computer, laptop computer, Personal Digital Assistant (PDA), or Internet-enabled cell phone. Note, however, that these and other particular examples of technologies provided herein are merely examples and do not constitute limitations of the present invention.

The client computer 104 includes a life expectancy client 106 which may, for example, be implemented using standalone software or a web browser. The user 102 may interact with the life expectancy client 106 using any suitable input devices, such as a mouse or keyboard, and using any software interface.

The system 100 also includes a server computer 108 having a life expectancy server 110. The life expectancy server 110 may be implemented, for example, in any kind of software executing on the server computer 108. The life expectancy server 110 may, for example, be implemented as or include a web server for providing content to the life expectancy client 106 in the form of web pages. The life expectancy client 106 acts as an intermediary between the user 102 and the life expectancy server 110 in ways that will be described in more detail below. The client computer 104 and server computer 108 may communicate over any kind of connection, such as the public Internet or a private intranet. Furthermore, those having ordinary skill in the art will appreciate that the functions performed by the client computer 104 and server computer 110 may be further subdivided into further computers or combined into a single computer.

The life expectancy system 100 may be implemented in any of a variety of ways. For example, in one embodiment of the present invention, a MySQL backend database at the server computer 110 drives PHP files that display content to the user 102. The backend database stores all of the questions in the questionnaire 114, the related information 134, and all other information not related to individual users. Another database stores user-specific information, such as the information stored in the user database 112, which may include, for example, the responses 118 provided by users.

The following description involves an example in which the user 102 maintains an account with the life expectancy server 110 and in which the life expectancy server 110 provides the user 102 with access to the account through a web-based interface. The life expectancy server 110 includes a user database 112 that includes account information for all users, such as usernames, passwords, names, and addresses. Note, however, that the use of user accounts is not a requirement of the present invention. Rather, the techniques disclosed herein may be implemented in systems in which users do not have, or are not required to have, accounts.

Assuming that the user 102 already has an account with the life expectancy server 110, the user 102 begins by logging in to his or her account (FIG. 2, step 202). The user 202 may log in by directing the life expectancy client 106 to the web site of the life expectancy server 110 (e.g., livingto100.com) and providing the user's username and password. The life expectancy server 110 may use well-known techniques to authenticate the user 102 and retrieve information about the user 102 from the user database 112.

The life expectancy server 110 includes a questionnaire 114 containing questions for soliciting health-related information from the user 102. Examples of the questionnaire were provided above, but the invention is not limited to use with those particular examples. The life expectancy server 110 provides the user 102 with individual questions 116 from the questionnaire 114, through the intermediary of the life expectancy client 106 (step 204). The user 102 provides the life expectancy server 110 with responses 118 to the questions 116, again through the intermediary of the life expectancy client 106 (step 206).

The life expectancy client 106 may provide the questions 116 to the user 102 in any way, such as by displaying each of them to the user 102 on an individual web page. Such web pages may include any mechanisms for receiving responses 118 from the user 102, such as text boxes for receiving typed text from the user 102, radio boxes, check boxes, or drop-down menus.

In the simplest case, questions 116 in the questionnaire 114 are provided to the user 102 sequentially. The questionnaire 114 may, however, be structured in any way to enable the questions 116 to be provided to the user 102 in any order. The life expectancy server 110 may, for example, choose which of the questions in the questionnaire 114 to provide next to the user 102 based on one or more of the user's previous responses 118. Furthermore, the life expectancy server 110 may choose questions to provide to the user 102 based on information about the user 102 stored in the user database 112. For example, the user database 112 may include previously-obtained health-related information about the user 102, such as the user's age and/or gender, which the server 110 may use to select questions to provide to the user 102.

As a result, the life expectancy server 110 need not provide all of the questions in the questionnaire 114 to the user 102. Rather, the questions 116 provided to the user may constitute a subset of the questions in the questionnaire 114. As this implies, different users may be provided with different questions from the questionnaire 114, and if the same user 102 responds to the questionnaire 114 multiple times, the user 102 may be provided with different questions each time.

Note further that although the questionnaire 114 is illustrated in FIG. 1 as a single questionnaire, this is not a requirement of the present invention. Rather, the life expectancy server 110 may include or have access to multiple questionnaires. For example, there may be distinct questionnaires tailored to different classes of users, such as users of different ages, occupations, or risk categories.

The life expectancy server 110 also includes a life expectancy calculator 120. Once the user 102 has responded to the questionnaire 114, the life expectancy calculator 120 identifies an estimate 122 of the user's life expectancy (step 208) and provides the user 122 with the estimate 122, through the intermediary of the life expectancy client 106 (step 210). Examples of techniques that may be used by the life expectancy calculator 120 to identify the life expectancy estimate are disclosed in the above-referenced book entitled Living To 100. For ease of illustration, the life expectancy estimate 122 and further information provided by the life expectancy server 110 are shown in FIG. 1 as being provided directly to the user 102, even though such information may be provided to the user 102 indirectly through the life expectancy client 106.

The life expectancy server 110 may further include an advisor module 124 that provides the user 102 with advice 126 for improving (increasing) the user's life expectancy based on the user's responses 118 to the questionnaire 114. In one embodiment of the present invention, the user 102 is provided with the life expectancy estimate 122 for free but is required to pay a fee to receive the life expectancy advice 126. This dual pricing structure provides the life expectancy calculator 120 with a practical commercial utility that is beneficial to the operator of the life expectancy server 110 because the free provision of the life expectancy estimate 122 may entice the user 102 to pay the required fee for the additional health-related advice 126, whereas the user 102 may not have been interested or willing to pay for such advice 126 absent the free life expectancy estimate 122.

Upon providing the user 102 with the life expectancy estimate 122, for example, the life expectancy server 110 may prompt the user 102 to provide payment to receive the life expectancy advice 126 (step 212). The prompt may, for example, take the form of a message explaining to the user 102 that the advice 126 is available for an additional fee, and provide the user 102 with a means (such as “accept” and “reject” buttons) for accepting or rejecting the fee-based advice 126.

If the user 102 indicates a desire to receive the advice 126 (step 214), the life expectancy server 110 may verify that the user 102 has provided sufficient payment (step 216) in any of a variety of ways that are well-known to those having ordinary skill in the art. For example, the life expectancy server 110 may access an external payment server 128, such as those provided by commercial services such as Verisign and PayPal, to process the user's payment. The user 102 may, for example, be redirected to a web page through which the user 102 provides payment information 130, such as a name, credit card number, credit card expiration, and payment amount. The payment server 128 performs all necessary functions, such as authenticating the user 102 transferring funds from the user 102 to the operator of the life expectancy server 110 (step 218).

The payment server 128 notifies the life expectancy server of a payment decision 132 indicating whether the user's payment has succeeded. If the user 102 chose not to receive the fee-based advice 126 or the user's payment failed, the life expectancy server 110 does not provide the user 102 with the life expectancy advice 126. Those having ordinary skill in the art will appreciate that payment from the user 102 to the operator of the life expectancy server 110 may be performed in any of a variety of other ways.

The life expectancy server 110 may include or have access to additional related information 134 that the life expectancy server 110 may provide to the user 102. Such information may, for example, include advertisements provided by third party businesses. As will be described in more detail below, such businesses may pay a fee to the operator of the life expectancy server 110 for the privilege of having such advertisements provided to the user 102 and to other users of the system 100. Although the life expectancy server 110 may be configured to provide such related information 134 to the user 102 in exchange for a fee paid by the user 102, in the embodiment illustrated in FIGS. 1 and 2, the related information 134 is provided to the user 102 for free.

More specifically, the life expectancy server 110 includes a tailoring module 136 that tailors some or all of the related information 134 for the user 102 based on the responses 118 provided by the user 102 to the questionnaire 114 (step 220), and provides the tailored information 138 to the user 102 (step 222). The tailored information 138 may, for example, be advertisements that were chosen based on a conclusion that such advertisements are likely to appeal to the user 102 based on the user's life expectancy, current age, current health conditions, or current activities. This provides the life expectancy calculator 120 with a further practical commercial utility.

If, for example, the advisor module 124 advises the user 102 to change to a vegetarian diet, the tailoring module 136 may provide user-specific information 138 to the user 102 containing advertisements for vegetarian food products and vegetarian supermarkets. Such targeted advertising may command premium rates due to its higher expected rate of success in producing customers for advertisers. Similarly, the tailoring module 136 may provide links to the web sites of businesses for a fee, instead of or in addition to providing advertisements directly on the website of the life expectancy system 100.

As the foregoing examples illustrate, embodiments of the present invention may be used to: (1) analyze various medical information, health-related behavior, and family history for various illnesses and longevity data in order to determine the user's life expectancy; (2) based upon the person's answers to the questionnaire 114, provide the user 102 with tailor-made feedback (in the form of the advice 126) about what the user 102 is already doing right (i.e., to increase his or her life expectancy), what he or she is already doing wrong (i.e., to decrease his or her life expectancy), and which determinants of life expectancy they cannot modify (such as family history); (3) provide (as part of or in addition to the tailor-made feedback) user-specific information 138, such as advertisements and links to other websites, to help the user 102 improve upon the factors for which a difference can be made; and (4) provide the user 102 with the ability to store his or her responses 118 and life expectancy 122 in the database 112, and compare their estimated life expectancy results from one time period of their choosing to another. For example, the system 100 may send a reminder (such as by email) to the user 102 periodically (e.g., every six months) to remind the user 102 to generate an updated life expectancy estimate, which may be stored in the system 100. The user 100 may then compare the estimated life expectancy results produced at these different points in time.

The system 100 may be provided directly over the public Internet for use by individual users. The system 100 may also, however, be provided and optionally customized for use by entities such as insurance companies, corporate human resources departments, health maintenance organizations (HMOs), physician provider organizations (PPOs), or other health care provider (HCP) to provide for use by their members or other internal users, such as their employees and customers. The terms “members” and “internal users” are used interchangeably herein to refer to any users, such as employees, customers, or patients of an entity to whom the entity has the authority to grant access to the system 100. Such authority may, for example, be granted to the entity by agreement with the operator of the system.

Such entities may either incorporate the system 100 into their own web sites or provide their internal users with access to the system 100 on an external web site. The operator of the system 100 may charge a fee to the entity and allow internal users of the entity to use the system 100 without paying additional individual fees. Such entities may thereby use the system 100 to provide their internal users with feedback that is specific to each user and also to provide such users with information and links for and to services and resources that the entity would like its users to have access to and to use. The payment scheme just described provides a practical commercial utility to the operator of the system 100 because transaction costs are lowered by charging a single (large) fee to the entity rather than charging multiple (small) fees to internal users of the entity.

In the embodiment just described, the user-specific information 138 may include information related to the entity; hyperlinks to external information; incentives and reimbursements regarding services such as specific health-care providers and pharmacies; alternative health resources such as massage, physical, psychological and occupational therapy, smoking, alcohol and illicit drug cessation services; exercise programs and facilities; and stress management programs. The system 100 may also provide the entity with the ability to keep track of which websites users of the system 100 visit while using the system 100.

Furthermore, the life expectancy system 100 may be adapted to the needs of specific subgroups of users, such as those with specific illnesses (e.g., diabetes, heart disease, stroke, Alzheimer's disease, or chronic kidney disease), or be combined with other calculators, such as financial expectancy calculators that assist people in predicting how long their financial resources will last and provide financial advice.

Various embodiments of the present invention, in summary, provide a lay-person-friendly technology that promotes healthy behaviors in a user-specific and interesting manner in such a way that enables users to actually effect changes that may lead to improved health and life expectancy. Furthermore, such a system may be constructed in a manner that provides monetary incentives to the developer to maintain the website, expand its utility to its users, and regularly update its content.

The life expectancy web site may be a standalone website associated with the operator of the life expectancy system 100. Alternatively, however, the life expectancy system 100 may be provided as a branded web site bearing the brand of a business that pays a fee to the owner of the life expectancy system 100 for such branding privileges. A business may find it commercially beneficial to use such a branded web site, for example, to draw customers to the business' primary commercial web site. Alternatively, the business may find it beneficial to provide such a branded web site to its clients and/or employees to use for the purpose of generating goodwill. The owner of the life expectancy system 100 may find a practical commercial utility in the ability to generate revenue from providing branded life expectancy web sites to multiple other businesses.

In another embodiment of the present invention, the owner of the life expectancy system 100 charges a fee to entertainment entities such as television, radio, documentary, and movie production companies or book publishers and the like to use the life expectancy system 100 in the production of programs and other media in which the calculation of life expectancy with or without the provision of feedback plays a role. For example, a television production company may pay a licensing fee to the life expectancy system owner for the right to use the life expectancy system in a reality television show in which contestants are provided with their estimated life expectancy (which for some, because of poor health habits, may be quite low) and then. The show may then use the advice and other information provided by the system 100 to help the contestants attempt to improve their health habits and life expectancy. The life expectancies of the contestants may then be re-estimated. Such entertainment may result in related products and services, such as self-help guides, books about the contents, DVDs containing software with the life expectancy calculator, and so on.

In general, various embodiments of the present invention have the benefit of providing the user 102 not only with an estimate 122 of the user's life expectancy, but also with comprehensive advice and information intended to be helpful to the user 102 in increasing the user's life expectancy 122. The system 100 is further beneficial to the system's operator because it enables the operator to obtain revenue from various parties, such as the user 102 and entities providing the related information 134 (e.g., advertising), as part of mutually-beneficial relationships.

More specifically, it was described above that the techniques disclosed in U.S. Pat. Nos. 5,937,387 and 6,269,339 calculate a user's “physiological age” and provide the user with various forms of feedback based on that physiological age. Physiological age, however, has no known well-accepted scientific basis, and there are no known dependable biomarkers of aging or physiologic age. Life expectancy, in contrast, is a scientifically valid and well-accepted demographic measure. Embodiments of the present invention, therefore, which use estimates of life expectancy, have practical utility in assisting health care providers and the lay public to gauge the impact of a person's health-related behaviors, environment, and other factors that they have no control of (such as familial patterns of disease and longevity) upon their life expectancy.

Unlike the techniques disclosed in U.S. Pat. No. 6,206,829, embodiments of the present invention do not attempt to diagnose or treat users of the system, nor are they meant to replace in-person assessment and advice from a health-care professional. Embodiments of the present invention, ratheri estimate a user's life expectancy and provide suggestions for how the user can improve that life expectancy.

Unlike the techniques disclosed in U.S. Pat. No. 6,334,192, embodiments of the present invention do not attempt to provide a level of risk for specific diseases. Rather, embodiments of the present invention provide an estimate of the user's age of death based upon the user's responses to a questionnaire.

Unlike the techniques disclosed in U.S. Pat. No. US 2003/0074226 A1 and U.S. Pat. No. 6,584,446, which discloses calculating life expectancy to assess insurability of insurance products, embodiments of the proposed invention predict life expectancy for the purpose of providing health-related advice intended to increase the user's life expectancy.

Unlike the techniques disclosed in U.S. Pat. No. 2003/0101075 A1, the purpose of which are to assist the care provider with provision of care, embodiments of the present invention provide advice to the user directly. Furthermore, the former utilizes medical bill data, while embodiments of the present invention rely upon the responses of the user. Moreover, the former provides algorithms of care plans to the health care provider, while embodiments of the present invention provide advice that includes referrals to other resources that may augment the advice provided. Still further, the former calculates remaining years of healthy life, while embodiments of the present invention calculates life expectancy (the same as age of death).

Unlike U.S. Pat. No. 2003/0208385 A1, the purpose of which is to provide advice for insurance underwriting purposes, embodiments of the present invention are used to provide health related advice directly to the user.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, Elements and components described herein may be further divided into additional components or joined together to form fewer components for performing the same functions.

Although certain embodiments of the present invention may be described as being implemented in a networked computer environment, this is not a requirement of the present invention. Rather, embodiments of the present invention may be implemented using a standalone computer or any configuration of any number of computers connected together in any way. For example, although certain embodiments of the present invention may be described herein as being implemented using the public Internet, this is not a requirement of the present invention. Embodiments of the present invention may, for example, be implemented using private internets or intranets. Although a client-server architecture is shown and described with respect to FIG. 1, embodiments of the present invention may use other architectures, such as providing a DVD that contains the necessary programming and feedback and thus negating the need for access to the internet or other network.

Various references are made herein to the “operator” of the life expectancy system 100 as the entity that charges a fee for use of the system 100. The term “operator” is used generally herein to refer to any entity (e.g., individual person or corporate entity) that has legal authority to charge a fee for use of the system 100 or any part thereof. The “operator” of the system 100 may, therefore, be the developer, owner, licensee, or purchaser of the system 100, by way of example.

The life expectancy calculator 120 is not limited to identifying the life expectancy estimate 122 by “calculating” the estimate 122. Rather, the calculator 120 may identify the estimate 122 in any way, such as by using lookup tables, rules, calculations, or any combination thereof.

The responses 118 provided by the user 102 may include any combination of health-related information, such as the user's behavior, environment, social relationships, demographic characteristics, psychological characteristics, and familial relations.

The advice 126 provided by the advisor module 124 is not limited solely to specific actions for the user 102 to take, but more generally may include any advice and/or information that is intended to help the user 102 to understand factors that may increase or decrease the user's life expectancy. The advice 126 may, for example, be age- and gender-specific advice regarding a plan of prevention and health screening to be carried out with the user's health-care provider. Such advice 126 may address individual ones of the user's responses 118 or any combination of such responses. For example, if the user 102 provides a response indicating that the user 102 smoke, the advice 126 may include a specific recommendation to the user 102 not to smoke. The advice 126 may, however, include more general advice in response to multiple ones of the responses 118.

The related information 134 may include any information related to the life expectancy 122 of the user 102, such as hyperlinks to related websites.

The advice 126 may be one-time advice provided to the user 102 in exchange for a fee. The advice 126 may, however, be recurring advice provided to the user 102 over time in exchange either for a one-time fee or a recurring fee. For example, the user 102 may pay a monthly or other periodic subscription fee to receive periodic updates to the advice 126 by email or other communications means. The advice 126 may be updated in any of a variety of ways, for example based on new medical developments, findings about life expectancy, or changes in the user's age or other characteristics. For example, the advice 126 may be updated when the user 102 reaches age 65 or some other milestone relevant to life expectancy.

The techniques described above may be implemented, for example, in hardware, software, firmware, or any combination thereof. The techniques described above may be implemented in one or more computer programs executing on a programmable computer including a processor, a storage medium readable by the processor (including, for example, volatile and non-volatile memory and/or storage elements), at least one input device, and at least one output device. Program code may be applied to input entered using the input device to perform the functions described and to generate output. The output may be provided to one or more output devices.

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by a computer processor executing a program tangibly embodied on a computer-readable medium to perform functions of the invention by operating on input and generating output. Suitable processors include, by way of example, both general and special purpose microprocessors. Generally, the processor receives instructions and data from a read-only memory and/or a random access memory. Storage devices suitable for tangibly embodying computer program instructions include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive programs and data from a storage medium such as an internal disk or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

The following is a description of techniques that may be used to implement a web site that generates an estimate of life expectancy and provides health-related advice based on that estimate according to one embodiment of the present invention. The following description is copyright 2005 by Thomas Perls MD, MPH, and is subject to the copyright notice above.

Content:
1. Intro
2. Questionnaire (Total of ˜50 questions)

CATEGORIES:
A: Personal (11 questions)
B: Lifestyle Habits & Environment (10 questions)
C: Nutrition & Exercise (10 questions)
D: Medical Check-up (7 questions)
E: Family History (4 questions+a 5^{th} question added for women age 35 and older)
3. Rules/Answer Key
4. Feedback
5. About the calculator

[Introduction]

Good health is an essential part of good living. An important step in keeping your body healthy and functional is to regularly evaluate how you are doing. Developed by Thomas Perls MD, MPH, Associate Professor of Medicine and Director of the New England Centenarian Study (www.bumc.bu.edu/centenarian) at Boston University Medical Center, The Livingto100 Life Expectancy Calculator is a great tool to quickly assess what you are doing right and what you are doing wrong and to show where your biggest health risks are. In terms of living to older age in good health, there is actually a great deal you can do! This tool will tell you your approximate life expectancy, and it will give you the opportunity to go back and change your answers to see how changes in your behavior might affect your life expectancy.

The information found on this website is designed to support, not replace the relationship between patient and physician

Take a few minutes and get an update on your health!

The Livingto100 Life Expectancy Calculator Web Site is committed to protecting your privacy. Please read our privacy statement.

By checking this box, I indicate that I have read and understand the Livingto100 Web Site privacy statement and that I wish to proceed with providing my private information in order to take advantage of the various features and services provided by this Web Site □

Home Page|Privacy Statement|Legal Statement|Disclaimer Advertising Policy

A. PERSONAL
1. What is your gender?
   [Checkboxes: male/female]
   Male (0)
   Female (0)
2. Enter your birth date:
   [Month/day/year]*
      * [The calculator should calculate the age]
3. What is your marital status?

[Dropdown: single, married, divorced/widowed, I feel I'm too young to get married]

If Male:
single (−5)
married (+7)
widowed/divorced (−5)
I feel I'm too young to get married (0)

If Female:
single (+3)
married (0)
widowed/divorced (0)
I feel I'm too young to get married (0)
4. Do your family members—other then your spouse and children—live near enough to spontaneously drop by?
a. Yes (+5)
b. No (−4)
c. I don't have any family (−2)
5a. How do you evaluate your current stress level (within the last 12 months)?
a. Low (0)
b. Medium (−2)
c. High (−4)
5b. How do you usually cope with your stress?
a. Very well! It helps me to get motivated. (+5)
b. Good! I can shed stress by using techniques that reduce stress (meditation, exercise, etc.) (+5)
c. I am doing all right! I am trying to find ways to protect myself from it. (−3)
d. Not very good! Stress eats away at me and I can't seem to shake it off. (−7)

6. Below is a list of typical sources of stress. Check all that you feel are stressful to you.

[of course, don't include table format or 2^{nd} column which is only for scoring purposes]

| | Score |
|---|---|
| excessive worrying (what if I have a car | −1 |
| accident and die, what if my home burns | |
| down, what if my daughter fails school etc) | |
| crime or victim-related | −1 |
| Illness or death of a family member or | −1 |
| loved one | |
| A loved one currently in harm's way (e.g. | −1 |
| war, fighting crime etc) | |
| death or illness of a pet | −1 |
| marital stress | −1 |
| school | −1 |
| community or global social concerns (e.g. | −1 |
| poverty, war, environment, religion etc) | |
| feeling underpaid/undervalued | −1 |
| getting laid off or fired from a job | −1 |
| legal troubles | −1 |
| new responsibilities (baby, job-related, | −1 |
| caring for someone etc) | |
| health problems | −1 |
| living conditions | −1 |
| regular conflict(s) with people other than | −1 |
| family (e.g. colleagues, neighbors) | |
| regular family-related conflicts | −1 |
| running late | −1 |
| job | −1 |
| traffic jams | −1 |
| financial insecurity | −1 |
| hormonal fluxes | −1 |
| other that we missed but that you find | −1 |
| significant | |

Scoring: Determine the sum of the above scores and multiply times 6. The maximum score however can be only −24. Even if they check all of these off. ADDITIONALLY, IF THEY ANSWERED “A” TO QUESTION 5b (stress motivates them), THEN ANY NEGATIVE SCORE FROM THIS QUESTION BECOMES “0”
7a. How many hours of sleep do you usually get per night?
   a. 3 to 5 hours (−8)
   b. 5 to 8 hours (0)
   c. Over 8 hours (0)

7b. How would you characterize your sleep habits?
a. Very good! I sleep enough and through the night. (+5)
b. It varies. I have sleepless nights once in while. (0)
c. It's okay! My sleep could certainly be better. (−2)
d. Very bad! Every night. I have problems falling or staying asleep. (−4)

8. How many years of formal education have you had?
a. >18 years (University degree and higher) (+8)
b. 13-16 years (+5)
c. 8-12 years (+2)
d. <8 years (−5)

9. Number of Hours Per Week That You Work at Your Job(s) (including your commute)
a. 40 hours or less
b. 41-60 hours (more than 9 hours per day up to 12 hours per day)
c. 61-80 hours per week (more than 12 hours per day up to 16 hours per day)
d. More than 80 hours per week (more than 16 hours per day)
e. I am retired or I am not working

Scoring:
a. 0
b. −6
c. −6
d. −12
e. 0

10. Number of Days Per Week You Work
a. 5 days or less per week
b. 6 days
c. 7 days
d. I am retired or not working

Scoring:
a. 0
b. −12
c. −24
d. 0

11. Are you optimistic about your aging, or pessimistic?
a. I feel I am aging well and my older years will be fulfilling ones
b. I am dreading my older years
c. Something in between the above tow options

Scoring:
a. +24
b. −4
c. 0

B. Habits/Environmental Circumstances
1. How is the air quality (air pollution) where you live?
a. Very bad! (Industrial area/lots of smog) (−5)
b. Average! (Urban area/medium smog) (0)
c. Okay! (Suburbs/low smog) (+1)
d. Very good! (Country side/no smog) (+2)
2. How often do you put your seat belt on when you are in a car (either as driver or passenger)?
e. Always (0)
f. About 80% of the time (0)
g. Half of the time (−5)
h. Less than half of the time (−7)
3. How many cups of caffeinated coffee do you drink per day?
a. None (0)
b. 1 to 2 cups (−1)
c. Over 3 cups (−3)
4. How many cups of tea do you drink per day?
   A. NONE (0)
   B. 1-2 CUPS PER DAY (+2)
   C. 3-5 CUPS PER DAY (+5)
   D. 6-10 CUPS PER DAY (−3)

e. greater than 10 cups per day (−6)
5a. Do you smoke or are you exposed to second-hand smoke?
a. Yes (0)*
b. No (0)*
   *[We don't need a score for the above . . . use the below scores for smoking. It is a good question to set the stage]
5b. How often do you smoke or chew tobacco (cigarettes, cigars, pipe, chewing tobacco)?
   A. DAILY (−2)
   B. LESS THAN DAILY (−1)
   C. RARELY OR NEVER (0)

5c. How many cigarettes do you smoke a day?
a. none (0)
b. 1 cigarette to half a pack 6
c. 1 pack 36
d. 1 and a half packs 48
e. 2 packs 60
f. 3 or more packs 108

FOR SCORING MULTIPLY THE SCORES FOR THE ANSWERS IN 5b and 5c

5D. WHAT IS YOUR EXPOSURE TO CLOSE-PROXIMITY SECOND-HAND SMOKE? ANSWER EVEN IF YOU ALSO SMOKE.
a. Daily and prolonged (−5)
b. Not daily, but often and prolonged (−2)
c. At least once a week and prolonged (−1)
d. Rarely or never (0)
5e. If you smoked tobacco in the past, but no longer smoke:
(a) I did not smoke in the past (0)
(b) I have chronic lung disease from my past history of smoking (for example, resulting in shortness of breath with minimal exertion, requiring supplemental oxygen, or a diagnosis of emphysema or chronic obstructive lung disease (COPD) (−12)
(c) I quite and seem to have minimal or no lung problems (0)
6.a) How many days per week do you usually consume alcohol?*
a. I don't drink alcohol (0)
b. 1 or 2 days per week (1)
c. 3 to 5 days per week (3)
d. Every day of the week (5)
6.b) On the days when you drink alcoholic beverages (beer, wine, liquor and mixed drinks) how many glasses do you usually drink?*
a. I don't drink (0)
b. 1-2 (+2)
c. 3-4 (−5)
d. 5 or more (−10)
   [For this scoring, please multiply the score from 6a with the score in 6b.]
7. How often do you take an aspirin (eg. 81-325 mg)?
a. Never (0)
   B. OCCASIONALLY (0)
   C. FREQUENTLY (+2)
   D. EVERY DAY (+10)
8. Do you wear sunscreen (at least SPF 30) or protective clothing when you spend time in the sun?
a. Rarely or never (−3)
b. Sometimes (−1)
c. Most of the time (+1)
d. Always (+3)
9. Do you engage in risky sexual (unprotected) behavior and/or do you inject illegal drugs?
a. Never (0)
b. rarely (once a year or less) (−3)
c. Sometimes (few times a year) (−6)
d. Often (every few months) (−12)
e. Very often (once or more a month) (−18)
10. Besides brushing your teeth, do you also floss your teeth every day?
   A. YES (+5)
   B. NO (−7)

c. I have dentures (0)
C. Nutrition & Exercise

1. What's your physical constitution?
A. HOW MUCH DO YOU WEIGH?

[Text entry: pounds or kg]
B. HOW TALL ARE YOU?

[Text entry: feet & inches or centimeters]

c. Do you body build or strength train to the degree that your body mass index is high because of muscle, not because of fat?
(a) yes (+10)
(b) no (0)
   *[if they answer “yes” to 1c, then use the (+10) and do not use the score resulting from the BMI calculation (the calculation resulting from the numbers provided for 1a and 1b). If they answer “no” to 1c, then use the BMI calculation]
   Rule: Give option for pounds and feet/inches as well as kilograms and centimeters. Convert the lbs to kg (divide by 2.2) and the feet and inches to inches and then to meters . . . multiply inches by 0.0254 to get meters. Once you have Kg and meters, divide Kg body weight by meters height, squared.)
For BMI . . .
   BMI <17, (−5)
   BMI 17 to <25 (+10)
   BMI 25 to <26 (−5)
   BMI 26 to <29 (−7)
   BMI 29 to <34 (−10)
   BMI 34 to <39 (−24)
   BMI >39 (−60)

2. How many servings of processed meats or fast foods (like hot dogs, burgers, bologna, etc.) do you eat a week?
a. None (0)
b. 1-2 servings (−1)
c. 3-4 servings (−3)
d. Over 5 servings (−8)
2b. How do you barbecue fish, poultry, or meat?
a. I am a vegetarian. (0)
b. I never barbecue. (0)
c. I put aluminum foil on the grill. (0)
d. Lightly grilled (0)
e. Almost charred or charred. (−4)
3. How many servings of dairy products (milk, cheese, yogurt, etc.) do you eat per day? (Example for 1 serving: 1 cup of milk, 1 cup of yogurt, or ½ oz. cheese). Choose all that apply!
a. 0-1 servings (−2)
b. 2-3 servings (+3)
c. More than 3 servings (+3)
d. I also take supplemental calcium (+3)

IT IS OK FOR THEM TO CHOOSE BOTH C AND D BUT WHEN IT IS SCORED, THEY SHOULD RECEIVE ONLY 3 POINTS NOT 6. If they choose d and any other answer, they should get +3.
4a. Do you snack between meals?
a. Yes=0
b. No=0
4b. If you do snack between meals, generally which of the following are your snacks? Choose all that apply!
a. Fruit (+5)
b. Yogurt (+5)
c. Veggies (+5)
d. sweets (−5)
e. Bagels/chips (−5)
f. Crackers (−3)
g. Popcorn (+2)
h. “Healthy” drinks (e.g. fruit based, teas, etc.). (+3)
i. “Unhealthy” drinks (e.g. popular sodas) (−1)

They should be able to choose all that apply. Add the highest negative score to the highest positive score to come up with a net score. [After summing the above answers, the maximum negative score can be −8 (even if they score −20) and the maximum positive score can be +8 (even if they score +20)].
5. How often per week do you have red meat as your main course?
a. I don't eat red meat (+8)
b. I eat red meat 1-2 days per week (+4)
c. I eat red meat 3-5 days per week (−12)
d. I eat red meat 6-7 days per week (−24)
   [So, if they answered “vegetarian” or (a) to question 2b, they should not receive the 16 points here at this question]

6. How often to you eat sweets such as ice cream, cake/pie/pastry, or candy bars?
a. I avoid sweets (0)
b. 1-2 days per week (0)
c. 3-5 days per week (−6)
d. Once a day (−15)
e. More than once per day (−20)

7. What about carbohydrates like white bread or rolls, potatoes, French fries, pasta, white rice (basically anything white)? If one meal's worth of each one of these counted as 1 serving of simple carbohydrate, how many servings of these do you have?
a. 3 or more servings a day (−6)
b. 1-2 servings a day (−3)
c. 1 serving every other day (0)
d. 1 serving twice a week (+2)
e. 1 or fewer servings a week (+5)
8. Do you believe you have a diet that leads to weight gain?
a. I eat too much everyday, making it easy for me to stay overweight or to gain even more weight. (−15)
b. I have a diet in which I am loosing weight with a target of a healthy weight. (+5)
c. I am maintaining a healthy weight with the diet I currently have. (+5)

9. Do you take iron either as a supplement or part of a multivitamin?

a. Yes (−8)

b. No (0)

c. Yes, but it relieves symptoms related to my anemia (low blood count) or I am taking it temporarily after surgery (0)

10. How many days a week do you exercise (strength training, aerobic exercises or activities such as swimming, running, strenuous walking and biking) for at least 30 minutes a day?
a. 7 days per week=(+25)
b. 6 days per week=(+18)
c. 5 days per week=(+15)
d. 3 days per week=(+10)
e. 2 days per week=(+4)
f. 1 day per week=(+1)
g. I don't exercise=(−12)

D. Medical Check-up

[The following is not a question . . . it is where we provide the user with an assessment of their weight based upon their answer to questions 1a-1c from the previous section and if it poses a risk to their health]

1. Based on the height and weight that you have provided earlier, we have calculated your body mass index (an indicator of whether or not you are overweight) to be: ## KG/m2 This BMI indicates (for most people) that you are: [fill in the applicable word from right column of the table below]
##[The above answer comes from the calculation made from questions la and b from the previous section of the calculator. Scores assessed according to BMI were already taken into account in that section] Also note that I have added a beice that incorporates strength training/muscle building

| **Options: | You are: |
|---|---|
| User answered | “A healthy weight” because you |
| “yes” to question 1c | indicate that because of strength training |
| in previous section, | you are relatively lean. |
| indicating that | |
| their BMI is high | |
| because of weight | |
| training or body | |
| building | |
| BMI <17 | “Too thin”. |
| BMI 17 to <25 | “At a healthy weight”. |
| BMI 25 to <26 | “Moderately overweight”. |
| BMI 26 to <29 | “Significantly overweight, meaning |
| | that you are at significant risk for |
| | obesity-related illnesses such as heart |
| | disease, high blood pressure, stroke, |
| | diabetes and cancer”. |
| BMI 29 to <34 | “In a high risk overweight category, |
| | meaning you are at high risk for obesity- |
| | related illnesses such as heart disease, |
| | high blood pressure, stroke and cancer”. |
| BMI 34 to <39 | “In an extremely high risk overweight |
| | category, meaning you are at extremely |
| | high risk for obesity-related illnesses |
| | such as heart disease, high blood |
| | pressure, stroke and cancer”. |
| BMI >39 | “In the highest risk category, |
| | meaning you are at severely high risk for |
| | obesity-related illnesses such as heart |
| | disease, high blood pressure, stroke and |
| | cancer”. |

1. Do you have a bowel movement at least once every two days?
a. Yes (0)
b. No, I have a bowel movement less frequently then every two days (−4)
2. Female: Do you regularly screen for cancer (skin and breasts) by doing self-examinations?
2. Male: Do you regularly screen for cancer (skin and testicles) by doing self-examinations?
a. Yes (+8)
b. No (−8)
c. I am 20 years old or younger (0)
3. What is your cholesterol level?
a. HDL cholesterol (good cholesterol):
   I. Lower than 40 mg/dl (1.0 mmol/L) (−10)
   II. Higher than 40 mg/dl (1.0 mmol/L) (+10)
   III. I haven't checked it in the last 3 years (−2)
   IV. I have had the test done within the past 3 years but don't remember the results (−1)

b. LDL cholesterol (bad cholesterol):
I. Lower than 100 mg/dl (2.6 mmol/L) (+7)
II. Higher than 100 mg/dl (2.6 mmol/L) (−7)
III. I haven't checked it in the last 3 years (−2)
IV. I have had the test done within the past 3 years but don't remember the results (−1_)

c. Total Cholesterol level:
I. Lower than 180 mg/dl or 5 mmol/l (+3)
II. Higher than 180 mg/dl or 5 mmol/l (−8)
III. I haven't checked it in the last 3 years (−2)
IV. I have had the test done within the past 3 years but don't remember the results (−1)
4. What is your systolic and diastolic blood pressure? (The systolic pressure is always stated first and the diastolic pressure second. For example: 122/76 (122 over 76); systolic=122, diastolic=76). If you don't your blood pressure, go to option c.
a. Systolic blood pressure (higher value):**
   I. Lower than 85 (−5)
   II. 86-100 (+6)
   III. 101-129 (0)
   IV. 130-139 (−5)
   V. 140-189 (−12)
   VI. 190-210 (−24)
   VII. 211-230 (−48)
   VIII. Higher than 230 (−72)
   IX. I don't know (0)
b. Diastolic blood pressure (lower value):**
   I. Lower than 80 (0)
   II. 80-89: (−1)
   III. 90-105: (−6)
   IV. 106-115: (−12)
   V. Higher than 116: (−24)
   VI. I don't know (0)
      **MAXIMUM NEGATIVE SCORE FOR A and B CAN'T BE HIGHER THAN −72
c. I have not had my blood pressure checked in the past year (−6)
   [note that they should have checked options IX and VI for a and b above if they answer this one]
5. Do you know whether you have diabetes? What is your fasting blood sugar level?
   a. I have not had it checked in the past 3 years) (−2)
   b. No diabetes (<120) (0)
   c. 120-200 (−6)
   d. >200 (−18)

6. Have you had a heart attack (answer only one)?
a. No heart attack (0)
b. Yes, I had a heart attack more than 2 years ago, and I took action to reduce my risk factors for another one (regular exercise, stop smoking, lose weight, changed diet) (−4)
c. Yes, I had a heart attack within the past 2 years, and I took action to reduce my risk factors for another one (regular exercise, stop smoking, lose weight, changed diet) (−7)
d. Yes, I had a heart attack within the past 2 years, and I HAVE NOT taken action to reduce my risk factors for another one (regular exercise, stop smoking, lose weight, changed diet) (−15)

7. When did you have your last doctor's appointment for your regular medical check-up (which includes blood pressure check, age- and gender-appropriate screenings, immunizations, review of medical history, and analysis about your risk factors)?
a. My last preventive care visit was over 3 years ago. −5.
b. My last preventive care visit was between 1-3 years ago.
c. My last preventive care visit was between within the past year. +2

E. FAMILY HISTORY
1. HOW MANY MEMBERS OF YOUR IMMEDIATE FAMILY (PARENTS AND SIBLINGS) HAVE DIABETES OR HAVE HAD A HEART ATTACK?
   A. NONE (0)
   B. ONE (−2)
   C. TWO (−4)
   D. THREE AND MORE: (−6)
   e. Don't know (0)
2. Does cancer run in your family? How many non-smoking members of your immediate family (grandparents, parents, siblings) were diagnosed with cancer (other than benign skin cancers)?
   A. NONE (+5)
   B. ONE (−4)
   C. TWO (−6)
   D. THREE AND MORE: (−12)
   e. don't know (0)
3. How old and how healthy are/were your parents? For your mother and father give one answer each.
a) Mother:
   1. Under the age of 80/healthy (0)
   2. Age 80-89 and healthy (+6)
   3. AGE 90-94 AND HEALTHY (+12)
      4. AGE 95-99 NO MATTER WHAT HEALTH (+24)
      5. AGE100+, NO MATTER WHAT HEALTH (+60)
   6. MOTHER HAS OR DIED OF A SMOKING RELATED (CANCER, HEART ATTACK, STROKE,
      EMPHYSEMA) ILLNESS OR TRAUMA PRIOR TO AGE 80 (−1)
         7. Under the age of 80 and dependent on others (−8)
   8. Age 81-89 and dependent on others (0)
   9. Age 90-94, dependent on others (+5)
   10. Died before age 80 (not from trauma or accident, see option 6) (−12)
      11. Died age 80-89 (0)
      12. Died age 90-94 (+5)
      13. Died age 95-99 (+24)
      14. Died age 100 or older (+60)
      15. Adopted, don't know (0)
b) Father
   4. Under the age of 80/healthy (0)
   5. Age 80-89 and healthy (+6)
   6. AGE 90-94 AND HEALTHY (+12)
      4. AGE 95-99, NO MATTER WHAT HEALTH (+24)
      5. AGE 100+, NO MATTER WHAT HEALTH (+60)
   6. FATHER HAS OR DIED OF A SMOKING RELATED (CANCER, HEART ATTACK, STROKE,
      EMPHYSEMA) ILLNESS OR TRAUMA PRIOR TO AGE 80 (−1)
         7. Under the age of 80 and dependent on others (−8)
   8. Age 81-89 and dependent on others (0)
   9. Age 90-94, dependent on others (+5)
   10. Died before age 80 (not from trauma or accident . . . see option 6) (−12)
      11. Died age 80-89 (0)
      12. Died age 90-94 (+5)
      13. Died age 95-99 (+24)
      14. Died age 100 or older (+60)
      15. Adopted, don't know (0)

4. Grandparents: Did any of your grandparents or great-grandparents live to age 98 years or older?
a. Yes (+36)
b. No (0)

NOTE!!! THE MAXIMUM SCORE ATTAINABLE FOR THE SUM OF QUESTIONS 2 through 4 is +120 (even if they got 161!) THE MAXIMUM NEGATIVE SCORE IS −12
5. Women only: Regarding how old you were when you last had a child . . .
a. I have not had any children and am still having my periods (0)
b. How old were you when you last had a child without fertility technology assistance? ______ years

RESULTS:
If they answer age 35-39 or older, give (+5)
If they answer age 40-45, give (+10)
If they answer age >45, give (+15)
If they answer age 34 or younger, give (0)

c. I had no children, but I had my last period (or had a hysterectomy) at age ______ years

RESULTS:
If they answer age <40, give (−5)
If they answer age 40-55, give (0)
If they answer age 56-59, give (+10)
If they answer age 60 or greater, give (+15)

Livingto100 Life Expectancy Calculator: Rules for Health Quotient/Live Expectancy

Calculating the Health Score FOR MEN:

| | Min and | |
|---|---|---|
| | max age | Chance for improvement |
| Health Score for A: | | High −101 to +9 |
| (personal/psychol) | | Med +10 to +20 |
| | | Low +31 to +64 |
| Health Score for B: | | High −346 to −75 |
| (Habits/environ) | | Med −74 to −1 |
| | | Low 0 to +35 |
| Health Score for C: | | High −167 to −35 |
| (Nutrition/exercise) | | Med −34 to −1 |
| | | Low +0 to +48 |
| Health Score for D: | | High −137 to −50 |
| (Medical checkup) | | Med −49 to +9 |
| | | Low +10 to +36 |
| Health Score for E: | | High −30 to 0 |
| (Family History) | | Med +1 to +29 |
| | | Low +30 to +120 |
| Health Score Total: | 20 years | High −781 to −151 |
| | | Med −150 to +59 |
| | 110 years | Low +60 to +303 |

Calculating the Health Score FOR WOMEN:

| | Score | |
|---|---|---|
| Health Score for A: | | High −96 to +9 |
| (personal/psychol) | | Med +10 to +20 |
| | | Low +31 to +64 |
| Health Score for B: | | High −346 to −75 |
| (Lifestyle/environ) | | Med −74 to −1 |
| | | Low 0 to +35 |
| Health Score for C: | | High −167 to −35 |
| (Nutrition/exercise) | | Med −34 to −1 |
| | | Low +0 to +48 |
| Health Score for D: | | High −137 to −50 |
| (Medical checkup) | | Med −49 to + 9 |
| | | Low +10 to +36 |
| Health Score for E: | | High −35 to 0 |
| (Family History) | | Med +1 to +29 |
| | | Low +30 to +120 |
| Health Score Total: | | High −781 to −151 |
| | | Med −150 to +59 |
| | | Low +60 to +303 |

RULE/CALCULATION FOR LIVE EXPECTANCY:

LIVE EXPECTANCY: Calculating the score for the live expectancy:

When all the scores are inputted, the computer should add the negatives and positives together to get a net score (e.g. −30 plus +45=+15). Then that sum should be divided by 12 (e.g.=+1.25). That final quotient, negative or positive is then added to 85 years if the person is a man and 89 years if they are female. So at the end, a Life expectancy or health span (in years) is calculated.

Therefore:

If you are a man, the lowest calculated age would be 85 yrs−(781/12)=85-65 =20 yrs (yikes!), and the oldest would be 85 yrs +(303/12)=85+25=110 yrs

If you are a woman, the lowest calculated age would be 89 yrs−(781/12) yrs=89-65=24 years, and the oldest would be 89 yrs+(303/12)=89+25 yrs=114 yrs

LIVE EXPECTANCY: Calculated healthspan ends up being less than actual age:

I would like the person to have a spot to put in their actual age. If the calculator produces an age that is less than the actual age of the person, the calculator should give the message, along with the calculated age, stating that:

“If your calculated life expectancy falls below your actual age, then you must have spectacular genes that make up for some of the problem areas you noted in the quiz. Congratulations!”

LIVE EXPECTANCY: Once The Calculation Is Made:

Here is your calculated health span. Below, please find the reasoning behind the questions, which you answered that made your score less than it should be.

IT IS VERY IMPORTANT THAT THE PROGRAM GIVES THE PARAGRAPH TO MATCH THE USER'S RESPONSE!

<<IN GIVING THE FEEDBACK, USE THE SUGGESTED HEADINGS. THERE IS NO NEED TO STATE THEIR ANSWER AGAIN AND DO NOT GIVE THE POINT VALUE. I PLACE THOSE THERE ONLY FOR OUR REFERENCE AND TO BE SURE THAT THE FEEDBACK SECTION MATCHES THE QUIZ SECTION>>

A. PERSONAL
1. What is your gender? [HEADING SHOULD BE: “Gender”]

Male: As a man, compared to women, you likely need to be more diligent about good health habits. If they develop heart attack or stroke, men tend to do so about ten years earlier than women. The reason for how and why women have this advantage is unclear. One possibility is that women make much more estrogen than men and this hormone might be associated with some protective effect, though this has in no way been proven. Another possibility is that chronic iron deficiency (due to menstruation) gives a woman her advantage. Iron is critical to our cells' ability to produce age-accelerating free radicals that also predispose to heart disease, stroke, Alzheimer's disease and cancer. Men can “menstruate” every eight months by donating a pint of blood at their local hospital or other blood bank center. Eight weeks is the recommended period (no pun intended) of time between donations. Donating blood has certainly not been proven to improve cardiovascular risk, though the downside, while performing a good deed, would seem to be minimal.

Female: Being female gives you a head start in the longevity marathon. Women usually have heart attack or stroke about 10 years later than men and 85% of centenarians are women and 15% are men. After menopause (average age 51 years), this advantage seems to diminish and therefore some scientists speculate that estrogen plays a role in this advantage. Another theory holds that chronic iron deficiency (due to menstruation) gives a woman her advantage. Iron is critical to our cells' ability to produce age-accelerating free radicals that also predispose to heart disease, stroke, Alzheimer's disease and cancer.
2. Enter your birth date:
   [Month/day/year]*

If the user indicates an age within one of the following ranges, use the corresponding advice paragraph:

The following text should appear before the recommendations:

The following actions and potential problems to be checked for are general guidelines for the general public. There may be instances where a particular recommendation does not pertain to a particular patient because of their unusual circumstances. Furthermore, in some cases something might need to be checked or examined at a younger than usually recommended age because of a familial predisposition or predisposing environmental exposure or behavior. The following is not meant to be an all-inclusive list and it is wise to have a detailed discussion with your health care provider to come up with a plan of prevention and screening that fits your particular circumstances.

If male and age 12 to 18 years:

A teenager eh? Well, kudos to you that you have taken this quiz and are interested in how your day to day health behaviors can dramatically impact upon your current and long term health and quality of life! Your teenage years are a pretty vulnerable time. Some habits developed during this period of your life are likely to have a dramatic impact upon your future health and wellbeing. These are:

1. Have a healthy diet and level of exercise that keeps you lean. Being overweight or obese now markedly increases the chance that you will be the same for the rest of your life. Being overweight markedly increases your risk for most age-related diseases including heart disease and diabetes. Being lean actually decreases your risk!

2. Don't drink and drive or get in a car where there is a possibility the driver has been drinking or is high. Just call a taxi or a friend. The inconvenience is far better than being dead or in a wheelchair for the rest of your life. You do have a choice! Don't take a chance.

3. Unprotected sex. Are you beginning to see a theme here? The idea is to just get through these early teens and twenties without getting killed or causing irreparable changes to your life. At your age, those events would be AIDS, hepatitis C, and pregnancy. If you are thinking “what are the chances? It won't happen to me”, you might as well approach a cliff and ask yourself, “just how close can I get before I fall?”

3. Stay in school. Years of education have a dramatic impact upon current and future survival and life expectancy.

4. Don't smoke! While it may be hard to resist tobacco because smoking runs in your family or because your friends smoke, remember that smoking makes you age much faster, it can markedly impair your athletic performance, it is a major cause of lung cancer, heart disease and stroke, and it burns a hole in your wallet.

5. Address depression. The teens are a tough time . . . You are trying to find out who you are. When who you are and who you want to be and what your family, school and environment don't necessarily jive with that wish, it can all feel pretty awful. But did you know that these feelings, sometimes fleeting, sometimes constant, are really common. And there are people you can turn to help you get out of the rut.

A. Each year, with your heath care provider, be sure to cover the following.
Medical history and physical exam
Tobacco use
Diet and exercise counseling
Alcohol and substance abuse
Sex-related concerns
Behavioral/Developmental assessment
Eating disorders
Depression screen
Self examination counseling (e.g. skin and testicular exam)
Driver safety counseling (e.g. seat belt use, designated driver, assessment of driving safety record once you have your drivers license)

B. And, have the following checked by physical examination and/or laboratory evaluation annually:
Height
Hearing and vision
Scoliosis screen
Obesity screening and counseling (body mass index and waist size)
Blood pressure
Urine glucose (for diabetes)
From age 13 and on, examination for hernia and testicular cancer

C. Be sure that the following is performed regularly at the recommended intervals:
Tuberculin skin test (PPD) every 1-3 years depending upon your risk of being exposed to tuberculosis
Total blood cholesterol tested once in your teenage years, unless there is a family history of elevated cholesterol, which then might need to be tested and managed more frequently
Appropriate immunizations (besides childhood immunizations, hepatitis B series (needed once) and tetanus every 5 years)
Screen for sexually transmitted diseases as warranted depending upon behavior and exposures

If female and age 12-18 years:

A teenager eh? Well, kudos to you that you have taken this quiz and are interested in how your day to day health behaviors can dramatically impact upon your current and long term health and quality of life! Your teenage years are a pretty vulnerable time. Some habits developed during this period of your life are likely to have a dramatic impact upon your future health and wellbeing. These are:

1. Have a healthy diet and level of exercise that keeps you lean. Being overweight or obese now markedly increases the chance that you will be the same for the rest of your life. Being overweight markedly increases your risk for most age-related diseases including heart disease and diabetes. Being lean actually decreases your risk!

2. Don't drink and drive or get in a car where there is a possibility the driver has been drinking or is high. Just call a taxi or a friend. The inconvenience is far better than being dead or in a wheelchair for the rest of your life. You do have a choice! Don't take a chance.

3. Unprotected sex. Are you beginning to see a theme here? The idea is to just get through these early teens and twenties without getting killed or causing irreparable changes to your life. At your age, those events would be AIDS, hepatitis C, and pregnancy. If you are thinking “what are the chances? It won't happen to me”, you might as well approach a cliff and ask yourself, “just how close can I get before I fall?”

3. Stay in school. Years of education have a dramatic impact upon current and future survival and life expectancy.

4. Don't smoke! While it may be hard to resist tobacco because smoking runs in your family or because your friends smoke, remember that smoking makes you age much faster, it can markedly impair your athletic performance, it is a major cause of lung cancer, heart disease and stroke, and it burns a hole in your wallet.

5. Address depression. The teens are a tough time . . . You are trying to find out who you are. When who you are and who you want to be and what your family, school and environment don't necessarily jive with that wish, it can all feel pretty awful. But did you know that these feelings, sometimes fleeting, sometimes constant, are really common. And there are people you can turn to help you get out of the rut.

A. Each year, with your heath care provider, be sure to cover the following:
History and physical exam
Tobacco use
Diet and exercise counseling
Alcohol and substance abuse
Sex-related concerns
Behavioral/Developmental assessment
Eating disorders
Depression screen
Self examination counseling (e.g. skin and testicular exam)
Driver safety counseling (e.g. seat belt use, designated driver, assessment of driving safety record once you have your drivers license)

B. And, have the following checked by physical examination and/or laboratory evaluation annually:
Height
Hearing and vision
Scoliosis screen
Obesity screening and counseling (body mass index and waist size)
Blood pressure
Urine glucose (for diabetes)
Chlamydia, if sexually active
Once menses has begun, check hemoglobin or hematocrit for anemia and treat with supplemental iron if having significant symptoms (e.g. unusual exertion intolerance, shortness of breath or fatigue)

C. Be sure that the following is performed regularly at the recommended intervals:
Tuberculin skin test (PPD) every 1-3 years depending upon your risk of being exposed to tuberculosis
Total blood cholesterol tested once in your teenage years, unless there is a family history of elevated cholesterol, in which case, it then might need to be tested and managed more frequently
Appropriate immunizations (besides childhood immunizations, hepatitis B series (needed once) and tetanus every 5 years)
Screen for sexually transmitted diseases as warranted depending upon behavior and exposures

If male and age 19 to 30 years:

A. Each year, with your heath care provider, be sure to cover the following:
History and physical exam
Tobacco use
Diet and exercise counseling
Alcohol and substance abuse
Sex-related concerns
Vision screen and hearing test
Depression screen
Self examination counseling (e.g. skin and testicular exam)
Driver safety counseling (e.g. seat belt use, assessment of driving safety record)

B. And, have the following checked by physical examination and/or laboratory evaluation annually:
Obesity screening and counseling (body mass index and waist size)
Blood pressure
Total blood cholesterol if there is a family history of elevated cholesterol, in which case, it then might need to be tested and managed on a regular basis with diet, exercise and/or medication
Blood glucose (for diabetes)

C. Be sure that the following is performed regularly at the recommended intervals:
Tuberculin skin test (PPD) every 1-3 years depending upon your risk of being exposed to tuberculosis
Screen for sexually transmitted diseases (STD) if you are at risk. Risk factors include history of prior STD, new or multiple sex partners, inconsistent use of barrier contraceptives, use of injection drugs. STD tests may include HIV, syphilis, and gonorrhea.
Tetanus vaccination every five years and be sure you have had your hepatitis B series

If female and age 19 to 30:

A. Each year, with your heath care provider, be sure to cover the following:
History and physical exam
Tobacco use
Diet and exercise counseling
Alcohol and substance abuse
Sex-related concerns
Vision screen and hearing test
Depression screen
Driver safety counseling (e.g. seat belt use, assessment of driving safety record)
Self examination counseling (e.g. skin and breast exam)

B. And have the following checked by physical examination and/or laboratory evaluation annually:
Obesity screening and counseling (body mass index and waist size)
Blood pressure
Breast exam and mammogram
Total blood cholesterol if there is a family history of elevated cholesterol, which then might need to be tested and managed on a regular basis with diet, exercise and/or medication
Pap (Papanicolaou) smear for women who have a cervix (testing can be less than annual if recommended by physician). Yearly check for Chlamydia if you are sexually active.
Screen for sexually transmitted diseases (STD) if you are at risk. Risk factors include history of prior STD, new or multiple sex partners, inconsistent use of barrier contraceptives, use of injection drugs. STD tests may include HIV, syphilis, and gonorrhea.

C. Be sure that the following is performed regularly at the recommended intervals:
Tuberculin skin test (PPD) every 1-3 years depending upon your risk of being exposed to tuberculosis
Screen for ovarian cancer using transvaginal ultrasound and rectovaginal pelvic exam for women who are at risk for ovarian cancer (At risk for ovarian cancer means either (a) having a family history with at least one first-degree relative with ovarian cancer; a second relative, either first-degree or second-degree, with breast, ovarian, or non-polyposis colorectal cancer; or (b) Testing positive for a hereditary ovarian cancer syndrome.
Tetanus vaccination every five years and be sure you have had your hepatitis B series

If male and age 31-49 years

A. Each year, with your heath care provider, be sure to cover the following:
History and physical exam
Tobacco use
Diet and exercise counseling
Alcohol and substance abuse
Sex-related concerns
Vision screen and hearing test
Depression screen
Self examination counseling (e.g. skin and testicular exam)
Driver safety counseling (e.g. seat belt use, assessment of driving safety record)

B. And, have the following checked by physical examination and/or laboratory evaluation annually:
Obesity screening and counseling (body mass index and waist size)
Blood pressure
Prostate exam and serum prostatic specific antigen or PSA, after age 40, if there is a family history of prostate cancer, being of African-American descent, consumption of a high-fat diet, or having had a vasectomy.
Stool for any blood (requires a special test to detect trace, invisible amounts) after age 40 if there is any family history of colon cancer
Total blood cholesterol
Blood glucose (for diabetes)
Electrocardiogram (ECG) if you are at increased risk for heart disease (increased risk would be the case if you have two or more of the following: a family history of heart attack, elevated cholesterol, high blood pressure, diabetes or smoking)

C. Be sure that the following is performed regularly at the recommended intervals:
Tuberculin skin test (PPD) every 1-3 years depending upon your risk of being exposed to tuberculosis
Exercise treadmill test (ETT) if at increased risk for heart disease (increased risk would be the case if you have two or more of the following: a family history of heart attack, elevated cholesterol, high blood pressure, diabetes or smoking)
Tetanus vaccination every five years

If female and age 31-49 years

A. Each year, with your heath care provider, be sure to cover the following:
History and physical exam
Tobacco use
Diet and exercise counseling
Alcohol and substance abuse
Sex-related concerns
Vision screen and hearing test
Depression screen
Osteoporosis prevention counseling and calcium intake
Self examination counseling (e.g. skin and breast exam)
Driver safety counseling (e.g. seat belt use, assessment of driving safety record)

B. And have the following checked by physical examination and/or laboratory evaluation annually:
Obesity screening and counseling (body mass index and waist size)
Blood pressure
Breast exam and mammogram
Total blood cholesterol
Stool for any blood (requires a special test to detect trace, invisible amounts) after age 40 if there is any family history of colon cancer
Pap (Papanicolaou) smear for women who have a cervix (testing can be less than annual if recommended by physician)
Screen for sexually transmitted diseases (STD) if you are at risk. Risk factors include history of prior STD, new or multiple sex partners, inconsistent use of barrier contraceptives, use of injection-drugs. STD tests may include HIV, syphilis, and gonorrhea.

C. Be sure that the following is performed regularly at the recommended intervals:
Tuberculin skin test (PPD) every 1-3 years depending upon your risk of being exposed to tuberculosis.
Screen for ovarian cancer using transvaginal ultrasound and rectovaginal pelvic exam for women who are at risk for ovarian cancer (At risk for ovarian cancer means either (a) having a family history with at least one first-degree relative with ovarian cancer; a second relative, either first-degree or second-degree, with breast, ovarian, or nonpolyposis colorectal cancer; or (b) Testing positive for a hereditary ovarian cancer syndrome.
Exercise treadmill test (ETT) if at increased risk for heart disease (increased risk would be the case if you have two or more of the following: a family history of heart attack, elevated cholesterol, high blood pressure, diabetes or smoking)
Tetanus vaccination every five years

If male and age 50-64 years:

A. Each year, with your heath care provider, be sure to cover the following:
History and physical exam
Tobacco use
Diet and exercise counseling
Alcohol and substance abuse
Sex-related concerns
Vision screen and hearing test
Depression screen
Self examination counseling (e.g. skin exam)
Driver safety counseling (e.g. seat belt use, assessment of driving safety record)

B. And, have the following checked by physical examination and/or laboratory evaluation annually:
Obesity screening and counseling (body mass index and waist size)
Blood pressure
Prostate exam (and serum prostatic specific antigen or PSA, if there is a family history of prostate cancer, being of African-American descent, consumption of a high-fat diet, or having had a vasectomy).
Stool for any blood (requires a special test to detect trace, invisible amounts)
Total blood cholesterol (and specifically HDL and LDL cholesterol and triglyceride levels)
Blood glucose (for diabetes)
Electrocardiogram (ECG) if you are at increased risk for heart disease (increased risk would be the case if you have two or more of the following: a family history of heart attack, elevated cholesterol, high blood pressure, diabetes or smoking)

C. Be sure that the following is performed regularly at the recommended intervals:
Colorectal cancer screening
   Flexible Sigmoidoscopy, every five years
   Complete colon examination by colonoscopy, every 10 years (no need to perform sigmoidoscopy in the tenth year when colonoscopy also examines the sigmoid colon)
Tuberculin skin test (PPD) every 1-3 years depending upon your risk of being exposed to tuberculosis
Tetanus vaccination every five years
Exercise treadmill test (ETT) if at increased risk for heart disease or if otherwise felt to be warranted by your health care provider

If female and age 50-64 years:

A. Each year, with your heath care provider, be sure to cover the following:
History and physical exam
Tobacco use
Diet and exercise counseling
Alcohol and substance abuse
Sex-related concerns
Vision screen and hearing test
Depression screen
Osteoporosis prevention counseling and calcium and vitamin D intake
Self examination counseling (e.g. skin and breast exam)
Driver safety counseling (e.g. seat belt use, assessment of driving safety record)

B. And have the following checked by physical examination and/or laboratory evaluation annually:
Obesity screening and counseling (body mass index and waist size)
Blood pressure
Breast exam and mammogram
Stool for any blood (requires a special test to detect trace, invisible amounts)
Total blood cholesterol (and specifically HDL and LDL cholesterol and triglyceride levels)
Electrocardiogram (ECG) if you are at increased risk for heart disease (increased risk would be the case if you have two or more of the following: a family history of heart attack, elevated cholesterol, high blood pressure, diabetes or smoking)
Pap (Papanicolaou) smear for women who have a cervix (testing can be less than annual if recommended by physician)
Screen for sexually transmitted diseases (STD) if you are at risk. Risk factors include history of prior STD, new or multiple sex partners, inconsistent use of barrier contraceptives, use of injection drugs. STD tests may include HIV, syphilis, and gonorrhea.

C. Be sure that the following is performed regularly at the recommended intervals:
Colorectal cancer screening
   Flexible Sigmoidoscopy, every five years
   Complete colon examination by colonoscopy, every 10 years (no need to perform sigmoidoscopy in the tenth year when colonoscopy also examines the sigmoid colon)
Tuberculin skin test (PPD) every 1-3 years depending upon your risk of being exposed to tuberculosis
Exercise treadmill test (ETT) if at increased risk for heart disease or if otherwise felt to be warranted by your health care provider
Check for osteoporosis with urine test and DEXA scan. Determine the frequency of this test with your physician
Screen for ovarian cancer using transvaginal ultrasound and rectovaginal pelvic exam for women who are at risk for ovarian cancer (At risk for ovarian cancer means either (a) having a family history with at least one first-degree relative with ovarian cancer; a second relative, either first-degree or second-degree, with breast, ovarian, or nonpolyposis colorectal cancer; or (b) Testing positive for a hereditary ovarian cancer syndrome.

If male and age 65 years and older:

A. Each year, with your heath care provider, be sure to cover the following:
History and physical exam
Tobacco use
Diet and exercise counseling
Alcohol and substance abuse
Sex-related concerns
Vision screen and hearing test
Depression screen
Screen for cognitive function impairment
Screen for urinary incontinence
Screen for polypharmacy (multiple prescribed and over the counter medications and herbs)
Osteoporosis prevention counseling and calcium intake
Self examination counseling (e.g. skin exam)
Driver safety counseling (e.g. seat belt use, assessment of driving safety record)

B. And, have the following checked by physical examination and/or laboratory evaluation annually:
Obesity screening and counseling (body mass index and waist size)
Blood pressure
Prostate exam (and serum prostatic specific antigen or PSA, if there is a family history of prostate cancer, being of African-American descent, consumption of a high-fat diet, or having had a vasectomy).
Stool for any blood (requires a special test to detect trace, invisible amounts)
Total blood cholesterol (and specifically HDL and LDL cholesterol and triglyceride levels)
Blood glucose (for diabetes)
Electrocardiogram (ECG) if you are at increased risk for heart disease (increased risk would be the case if you have two or more of the following: a family history of heart attack, elevated cholesterol, high blood pressure, diabetes or smoking)

C. Be sure that the following is performed regularly at the recommended intervals:
Colorectal cancer screening
   Flexible Sigmoidoscopy, every five years
   Total colon examination by colonoscopy, every 10 years (no need to perform sigmoidoscopy in the tenth year when colonoscopy also examines the sigmoid colon)
Tuberculin skin test (PPD) every 1-3 years depending upon your risk of being exposed to tuberculosis
Tetanus vaccination every five years
Exercise treadmill test (ETT) if at increased risk for heart disease or if otherwise felt to be warranted by your health care provider
Obtain a flu shot during flu season (October-February)
Every ten years, receive a pneumococcal vaccination (against pneumonia)

If female and age 65 years and older:

A. Each year, with your heath cares provider, be sure to cover the following:
Medical history and physical exam
Tobacco use
Diet and exercise counseling
Alcohol and substance abuse
Sex-related concerns
Vision screen and hearing test
Depression screen
Screen for cognitive function impairment
Screen for urinary incontinence
Screen for polypharmacy (multiple prescribed and over the counter medications and herbs)
Osteoporosis prevention counseling and calcium and vitamin D intake
Self examination counseling (e.g. skin and breast exam)
Driver safety counseling (e.g. seat belt use, assessment of driving safety record)

B. And have the following checked by physical examination and/or laboratory evaluation annually:
Obesity screening and counseling (body mass index and waist size)
Blood pressure
Breast exam and mammogram
Stool for any blood (requires a special test to detect trace, invisible amounts)
Total blood cholesterol (and specifically HDL and LDL cholesterol and triglyceride levels)
Electrocardiogram (ECG) if you are at increased risk for heart disease (increased risk would be the case if you have two or more of the following: a family history of heart attack, elevated cholesterol, high blood pressure, diabetes or smoking)
Pap (Papanicolaou) smear for women who have a cervix (testing can be less than annual if recommended by physician)
Screen for sexually transmitted diseases (STD) if you are at risk. Risk factors include history of prior STD, new or multiple sex partners, inconsistent use of barrier contraceptives, use of injection drugs. STD tests may include HIV, syphilis, and gonorrhea.

C. Be sure that the following is performed regularly at the recommended intervals:
Check for osteoporosis with urine test and DEXA scan
Colorectal cancer screening
   Flexible Sigmoidoscopy, every five years
   Total colon examination by colonoscopy, every 10 years (no need to perform sigmoidoscopy in the tenth year when colonoscopy also examines the sigmoid colon)
Screen for ovarian cancer using transvaginal ultrasound and rectovaginal pelvic exam for women who are at risk for ovarian cancer (At risk for ovarian cancer means either (a) having a family history with at least one first-degree relative with ovarian cancer; a second relative, either first-degree or second-degree, with breast, ovarian, or nonpolyposis colorectal cancer; or (b) Testing positive for a hereditary ovarian cancer syndrome.
Tuberculin skin test (PPD) every 1-3 years depending upon your risk of being exposed to tuberculosis
Tetanus vaccination every five years
Exercise treadmill test (ETT) if at increased risk for heart disease or if otherwise felt to be warranted by your health care provider
Obtain a flu shot during flu season (October-February)
Every ten years, receive a pneumococcal vaccination (against pneumonia)
3. Marital Status

If male, single (−5), Interestingly, New England Centenarian Study data suggest that marriage or having a partner in your life has different effects upon your life expectancy depending upon whether you are a man or woman. As a man, being married will likely improve your chances of living to 100. Findings from other studies indicate that being single is not such a big deal, but if you were recently divorced, that

If male, married (+7), New England Centenarian Study data suggest that in the case of men, being married provides a survival advantage.

If male, widowed/divorced (−5) New England Centenarian Study data suggest that in the case of men, being married provides a survival advantage.

If male, I'm too young to get married (0) Too young to get married? Not too much to be said here regarding the impact of this on survival except that it is important to once again stress the dangers of unprotected sex. Otherwise, take advantage of this time to explore what are the characteristics in a life-long partner that are a good fit for you and of course, good luck in finding just the right person!

If female, single Interestingly, New England Centenarian Study data suggest that marriage or having a partner in your life has different effects upon your life expectancy depending upon whether you are a man or woman. In the case of a woman, being or having been married does tot provide a distinct survival advantage, at least in the ability to achieve exceptional old age. An unusual proportion of the female centenarians never married (about 15%). Perhaps personality features that lead to perseverance, independence, and assertiveness provide a survival advantage. Such women may also be particularly good at managing stress. On the other hand, nearly 100% of the male centenarians are married or were only recently widowed.

If female, married Interestingly, New England Centenarian Study data suggest that marriage or having a partner in your life has different effects upon your life expectancy depending upon whether you are a man or woman. In the case of a woman, being or having been married does not provide a distinct survival advantage, at least in the ability to achieve exceptional old age. An unusual proportion of the female centenarians never married (about 15%). Perhaps personality features that lead to perseverance, independence, and assertiveness provide a survival advantage. Such women may also be particularly good at managing stress. On the other hand, nearly 100% of the male centenarians are married or were only recently widowed.

If female, widowed/divorced Interestingly, New England Centenarian Study data suggest that marriage or having a partner in your life has different effects upon your life expectancy depending upon whether you are a man or woman. In the case of a woman, being or having been married does not provide a distinct survival advantage, at least in the ability to achieve exceptional old age. An unusual proportion of the female centenarians never married (about 15%). Perhaps personality features that lead to perseverance, independence, and assertiveness provide a survival advantage. Such women may also be particularly good at managing stress. On the other hand, nearly 100% of the male centenarians are married or were only recently widowed.

If female, I'm too young to get married Don't feel any pressure to get married with regard to improving your chances of living to 100. Interestingly, New England Centenarian Study data suggest that marriage or having a partner in your life has different effects upon your life expectancy depending upon whether you are a man or woman. In the case of a woman, being or having been married does not provide a distinct survival advantage, at least in the ability to achieve exceptional old age. An unusual proportion of the female centenarians never married (about 15%). Perhaps personality features that lead to perseverance, independence, and assertiveness provide a survival advantage. Such women may also be particularly good at managing stress. On the other hand, nearly 100% of the male centenarians are married or were only recently widowed. Otherwise, it is also important to once again stress the dangers of unprotected sex.
4. Proximity of Family
a. Yes (+5) Having reasonably frequent contact with family or friends who are like family to you can be an important feature of your ability to manage stress well and is probably a life expectancy extender.
   Good thing that you have family nearby. Extended family cohesiveness and frequent contact is a notable feature of centenarian families. Researchers have noted that people who do not belong to cohesive families have fewer coping resources and increased levels of social and psychological stress. Psychological stress is associated with heart disease, various cancers and increased mortality risk.

b. No (−4) Do what you can to foster meaningful relationships with family and/or friends who live near enough to you that they can spontaneously drop by. Having such people in your lives is conducive to a longer, healthier life and probably helps you manage stress better and enjoy life more.
Extended family cohesiveness and frequent contact is a notable feature of centenarian families. Researchers have noted that people who do not belong to cohesive families have fewer coping resources and increased levels of social and psychological stress. Psychological stress is associated with heart disease, various cancers and increased mortality risk. Moving close to family might not be feasible for you. Do what you can to increase the frequency of your and their visits and develop close and mutually supportive relationships with nearby friends.
c. I don't have any family (−2)

Extended family cohesiveness and frequent contact is a notable feature of centenarian families. Researchers have noted that people who do not belong to cohesive families have fewer coping resources and increased levels of social and psychological stress. Psychological stress is associated with heart disease, various cancers and increased mortality risk. Moving close to family might not be feasible for you. Do what you can to develop close and mutually supportive relationships with nearby friends.
5a. How do you evaluate your current stress level (within the last 12 months)?
a. Low (0)
b. Medium (−2)
c. High (−4)

No feedback given for the above question (5a)
5b. How Do You Cope With Stress?

a. Very well! It helps me to get motivated. (+5)

Excellent! Managing your stress in such a way that it doesn't eat away at you, and in your case actually acts as a motivator is tremendous. Your ability to manage stress effectively will substantially improve your life expectancy and increase the number of healthy years ahead of you.

b. Good! I can shed stress by using techniques that reduce stress (meditation, exercise, etc.) (+5)

Keep up the good work. Doing your best to better manage your stress will positively impact on many different aspects of your emotional and physical health. Consider numerous options in better managing your stress. Take a deep breath next time you are stressed and step back for a moment knowing that shedding the stress will be a much better immediate way of dealing with the matter and long term it will help you delay or even avoid illnesses such as heart disease, stroke, cancer, and Alzheimer's disease. Try to learn methods (Tai Chi, breathing techniques, meditation) or activities (physical exercise, prayer) that help with shedding stress instead of internalizing it.
Centenarians shed emotional stress exceptionally well. Their stress-shedding personalities and the familial support, which they receive and contribute to are important stress-reducing mechanisms. Refer to the Mind Body Institute for more information [http://www.mindbody.harvard.edu]

c. I am doing all right! I am trying to find ways to protect myself from it. (−3)

Keep up the good work. Doing your best to better manage your stress will positively impact on many different aspects of your emotional and physical health. Consider numerous options in better managing your stress. Take a deep breath next time you are stressed and step back for a moment knowing that shedding the stress will be a much better immediate way of dealing with the matter and long term it will help you delay or even avoid illnesses such as heart disease, stroke, cancer, and Alzheimer's disease. Try to learn methods (Tai Chi, breathing techniques, meditation) or activities (physical exercise, prayer) that help with shedding stress instead of internalizing it.
Centenarians shed emotional stress exceptionally well. Their stress-shedding personalities and the familial support, which they receive and contribute to are important stress-reducing mechanisms. Refer to the Mind Body Institute for more information [http://www.mindbody.harvard.edu]

d. Not very good! Stress eats away at me and I can't seem to shake it off. (−7)

Please make better management of your stress a top priority. Doing your best to better manage your stress will positively impact on many different aspects of your emotional and physical health. Consider numerous options in better managing your stress. Take a deep breath next time you are stressed and step back for a moment knowing that shedding the stress will be a much better immediate way of dealing with the matter and long term it will help you delay or even avoid illnesses such as heart disease, stroke, cancer, and Alzheimer's disease. Try to learn methods (tai chi, breathing techniques, meditation) or activities (physical exercise, prayer) that help with shedding stress instead of internalizing it.
Centenarians shed emotional stress exceptionally well. Their stress-shedding personalities and the familial support, which they receive and contribute to are important stress-reducing mechanisms. Refer to the Mind Body Institute for more information [http://www.mindbody.harvard.edu]6. Number of Stressors
FEEDBACK FOR: Number of Stressors In Your Life

If score is: >−30

You have a lot of significant stressors in your life that likely negatively impact upon your life expectancy and certainly your current quality of life. Of course, where you can, decrease or take steps to eventually remove the stress. Additionally, try a number of strategies that can reduce the adverse effect stress can have, on you. The key here is that you perform these regularly:
Take time out to take one or two deep breaths
Leave earlier to get to appointments on time
Plan awards for yourself when you accomplish things
Plan regular events that you can look forward to such as a sporting event, a concert series, a date and/or massage with your loved one
Regularly take time out for yourself for an activity that is not stressful or reduces stress even once a week (exercise, yoga, stretching, going for a hike or walk)
Don't be hard on yourself. You are your best friend.
Participate in activities that will lead to your laughing
Go to bed earlier, not later
Try something new and get good enough at it to the point that you enjoy it (art, a game, line dancing, a sport or recreation)
Stop behaviors that you personally have control of and ultimately make you feel bad (binge eating, smoking, etc)
Demonstrate and receive love for and from someone (including a pet)
Share your troubles and concerns with someone who you trust and who listens and do the same for them

FEEDBACK FOR: Number of Stressors In Your Life

If score is: −6 to −29

Thankfully, the number of major stressors in your life are minimal, though understandably, the few that you have may still have a significant impact upon your well-being. Of course, where you can, decrease or take steps to eventually remove the stress. Additionally, try a number of strategies that can reduce the adverse effect stress can have on you. The key here is that you perform these regularly:
Take time out to take one or two deep breaths
Leave earlier to get to appointments on time
Plan awards for yourself when you accomplish things
Plan regular events that you can look forward to such as a sporting event, a concert series, a date and/or massage with your loved one
Regularly take time out for yourself for an activity that is not stressful or reduces stress even once a week (exercise, yoga, stretching, going for a hike or walk)
Don't be hard on yourself. You are your best friend.
Participate in activities that will lead to your laughing
Go to bed earlier, not later
Try something new and get good enough at it to the point that you enjoy it (art, a game, line dancing, a sport or recreation)
Stop behaviors that you personally have control of and ultimately make you feel bad (binge eating, smoking, etc)
Demonstrate and receive love for and from someone (including a pet)
Share your troubles and concerns with someone who you trust and who listens and do the same for them

FEEDBACK FOR: Number of Stressors In Your Life

If score is: 0-5

It is wonderful that you have not noted significant stressors in your life. Alternatively, perhaps there are what would normally be regarded as stressors in your life, but you find they don't adversely affect you. Nonetheless, consider the below strategies for reducing the effect of stress. Adopting one or more of these that fit well for you might even add years to your life.
Take time out to take one or two deep breaths
Leave earlier to get to appointments on time
Plan awards for yourself when you accomplish things
Plan regular events that you can look forward to such as a sporting event, a concert series, a date and/or massage with your loved one
Regularly take time out for yourself for an activity that is not stressful or reduces stress even once a week (exercise, yoga, stretching, going for a hike or walk)
Don't be hard on yourself. You are your best friend.
Participate in activities that will lead to your laughing
Go to bed earlier, not later
Try something new and get good enough at it to the point that you enjoy it (art, a game, line dancing, a sport or recreation)
Stop behaviors that you personally have control of and ultimately make you feel bad (binge eating, smoking, etc)
Demonstrate and receive love for and from someone (including a pet)
Share your troubles and concerns with someone who you trust and who listens and do the same for them
7a. Hours of Sleep

a. 3 to 5 hours (−12)

Three to five hours of sleep a night is simply not enough sleep. Several studies, particularly a large study performed in Japan, indicates increased mortality and heart disease associated with an average of less than 6 hours of sleep a night.

b. 5 to 8 hours (0)

You are getting a reasonable amount of sleep. Several studies, particularly a large study performed in Japan, indicates increased mortality and heart disease associated with an average of less than 6 hours of sleep a night.

c. Over 8 hours (0)

You may be getting too much sleep. Interestingly, several studies, particularly those conducted in Japan, indicate an increased mortality among people getting, on average, more than 9 hours of sleep each night. This is a controversial enough finding that your life expectancy hasn't been shortened as a result of your answer. Perhaps your increased sleep duration is associated with an illness, medications or drugs and/or alcohol, some or all of which could be the cause of increased mortality risk. It may be worthwhile to discuss this issue with your doctor, especially if this is a relatively new phenomenon.

7b. Sleep Habits
a. Very good! I sleep enough and through the night. (+5)
It is terrific that you are getting adequate and quality sleep. Adequate sleep is also a sign of good health.

b. It varies. I have sleepless nights once in while. (0)

Not getting enough sleep or experiencing ineffective sleep is common. There are many causes of sleep-related disorders and of not getting enough effective sleep.
Not getting enough sleep or experiencing ineffective sleep is common. There are many causes of sleep-related disorders and not getting enough effective sleep. There are also numerous good approaches and treatments to reverse these problems. Unfortunately, there are ineffective and even harmful ways of attempting to cure sleep problems. Two helpful sites are the Stanford University Center for the Center of Excellence for the Diagnosis and Treatment of Sleep Disorders [http://www.med.stanford.edu/school/psychiatry/coe/] and sleepnet.com [http://www.sleepnet.com/].

c. It's okay! My sleep could certainly be better. (−2)

Not getting enough sleep or experiencing ineffective sleep is common. In your case however, trouble with sleep every night could indicate a serious but perhaps curable problem. You will probably have to go beyond your primary care physician for help with your sleep disorder.
Not getting enough sleep or experiencing ineffective sleep is common. There are many causes of sleep-related disorders and not getting enough effective sleep. There are also numerous good approaches and treatments to reverse these problems. Unfortunately, there are ineffective and even harmful ways of attempting to cure sleep problems. Two helpful sites are the Stanford University Center for the Center of Excellence for the Diagnosis and Treatment of Sleep Disorders [http://www.med.stanford.edu/school/psychiatry/coe/] and sleepnet.com [http://www.sleepnet.com/].

d. Very bad! Every night I have problems falling or staying asleep. (−4)

Not getting enough sleep or experiencing ineffective sleep is common. In your case however, trouble with sleep every night could indicate a serious but perhaps curable problem. You will probably have to go beyond your primary care physician for help with your sleep disorder.
Not getting enough sleep or experiencing ineffective sleep is common. There are many causes of sleep-related disorders and not getting enough effective sleep. There are also numerous good approaches and treatments to reverse these problems. Unfortunately, there are ineffective and even harmful ways of attempting to cure sleep problems. Two helpful sites are the Stanford University Center for the Center of Excellence for the Diagnosis and Treatment of Sleep Disorders [http://www.med.stanford.edu/school/psychiatry/coe/] and sleepnet.com [http://www.sleepnet.com/].

8. Education

a. >18 years (University degree and higher) (+8)

All those years of education will likely increase your life expectancy for obvious reasons such as your being a more informed consumer of health care and your being more likely to partake in healthy behaviors such as not smoking and having a healthy diet. Regarding your brain health, continue to participate in cognitively challenging activities, exercising parts of your brain that haven't been used so much new activities that are difficult (what neuropsychologist Paul Naussbaum terms “novel and complex”). Learn a new language or musical instrument; if you don't have time for these most potent activities, try crosswords, Scrabble, bridge, sculpture or paining . . . but when you get good at something, move on to another cognitively challenging activity. Cognitively challenging activities as an adult, have been shown to delay the onset of Alzheimer's disease and to slow its progression.

b. 13-16 years (+5)

Those added years of education will likely increase your life expectancy for obvious reasons such as your being a more informed consumer of health care and your being more likely to partake in healthy behaviors such as not smoking and having a healthy diet. Regarding your brain health, continue to participate in cognitively challenging activities, exercising parts of your brain that haven't been used so much new activities that are difficult (what neuropsychologist Paul Naussbaum terms “novel and complex”). Learn a new language or musical instrument; if you don't have time for these most potent activities, try crosswords, Scrabble, bridge, sculpture or paining . . . but when you get good at something, move on to another cognitively challenging activity. Cognitively challenging activities as an adult, have been shown to delay the onset of Alzheimer's disease and to slow its progression.

c. 8-12 years (+2)

Your education will likely increase your life expectancy for obvious reasons such as your being a more informed consumer of health care and your being more likely to partake in healthy behaviors such as not smoking and having a healthy diet. Regarding your brain health, continue to participate in cognitively challenging activities, exercising parts of your brain that haven't been used so much new activities that are difficult (what neuropsychologist Paul Naussbaum terms “novel and complex”). Learn a new language or musical instrument; if you don't have time for these most potent activities, try crosswords, Scrabble, bridge, sculpture or paining . . . but when you get good at something, move on to another cognitively challenging activity. Cognitively challenging activities as an adult, have been shown to delay the onset of Alzheimer's disease and to slow its progression.

d. <8 years (−5)

Years of education likely increases your life expectancy because more educated persons are generally more informed consumers of health care and more likely to partake in healthy behaviors such as not smoking and having a healthy diet. Despite the few years of education that you have had there are important things that you can do as an adult to delay or even escape age-related diseases that effect the brain. You should participate in cognitively challenging activities, exercising parts of your brain that haven't been used so much new activities that are difficult (what neuropsychologist Paul Naussbaum terms “novel and complex”). Learn a new language or musical instrument; if you don't have time for these most potent activities, try crosswords, Scrabble, bridge, sculpture or paining . . . but when you get good at something, move on to another cognitively challenging activity. Cognitively challenging activities as an adult, have been shown to delay the onset of Alzheimer's disease and to slow its progression.
9. Hours on the Job
   a. 40 hours or less (0)

A recent Japanese study of the relationship between work hours and heart attack risk reveled that men who worked, on average, 11 hours or more a day had twice the risk of heart attack. Interestingly though, is that those who worked less than 7 hours a day were also at increased risk. If you find your current workload comfortable and that you are taking advantage of your leisure time to perform healthy and enjoyable activities, good for you and keep up the good work!
b. 41-60 hours (more than 9 hours per day up to 12 hours per day) (−6)

A recent Japanese study of the relationship between work hours and heart attack risk reveled that men who worked, on average, 11 hours or more a day had twice the risk of heart attack. Interestingly though, is that those who worked less than 7 hours a day were also at increased risk. The number of hours you are working might translate into poorer health over a long period of time. Try to take steps to cut back. If you can't cut back, attempt to find time during the day for meaningful breaks.
c. 61-80 hours per week (more than 12 hours per day up to 16 hours per day) (−6)

A recent Japanese study of the relationship between work hours and heart attack risk reveled that men who worked, on average, 11 hours or more a day had twice the risk of heart attack. Interestingly though, is that those who worked less than 7 hours a day were also at increased risk. The number of hours you are working is way out of the ordinary. If you continue to work at this rate, you may be really setting yourself up for chronic medical problems. Try to take steps to cut back. If you can't cut back, attempt to find time during the day for meaningful breaks.
d. More than 80 hours per week (more than 16 hours per day) (−12)

A recent Japanese study of the relationship between work hours and heart attack risk reveled that men who worked, on average, 11 hours or more a day had twice the risk of heart attack. Interestingly though, is that those who worked less than 7 hours a day were also at increased risk. The number of hours you are working is way out of the ordinary. If you continue to work at this rate, you may be really setting yourself up for chronic medical problems. Try to take steps to cut back. If you can't cut back, attempt to find time during the day for meaningful breaks.

e. I am retired or not working (0)

You indicated that you are not employed. Hopefully that means you are retired and pursuing what makes you happy and fulfilled. Otherwise, being unemployed may be a significant stress for you that the quiz attempts to capture in its question about typical stressors.
10. Number of Days Per Week You Work
   a. 5 days or less per week

You are working the usual number of days per week. Those who regularly work 6 or 7 days a week probably set themselves up for stress-related chronic illness(es)
b. 6 days

Working 6 days a week is too much and is likely setting you up for stress-related chronic illness. Do what you can to back off to 5 days a week.
c. 7 days

Working 6 days a week is too much and is likely setting you up for stress-related chronic illness. Do what you can to back off to 5 days a week.
d. I am retired or not working

[IF A PERSON SELECTS THIS OPTION, DON'T PROVIDE THE ABOVE HEADING AND FEEDBACK]
11. Optimism
   a. I feel I am aging well and my older years will be fulfilling ones

Your optimism likely has a real impact upon our longevity! According to AARP and Dutch investigators who, over a decade, tracked 1,000 people ages 65 to 85, people who are open to opportunities and possibilities have a 55 percent lower risk of death; in doing so, you end up less stressed, happier, healthier and more long-lived.

b. I am dreading my older years

Your pessimism about your aging might independently decrease your longevity. Maybe your pessimism is based upon some inaccurate assumptions about aging. Ideas like “the older you get, the sicker you get”, or “it's all downhill from here” are simply false. And, as discussed elsewhere in this feedback, there is a lot you can do about how you age. One of the things you can do, is alter your attitude. According to AARP and Dutch investigators who, over a decade, tracked 1,000 people ages 65 to 85, people who are open to opportunities and possibilities have a 55 percent lower risk of death; in doing so, you end up less stressed, happier, healthier and more long-lived.

B. Lifestyle Habits/Environmental circumstances 1. Smog

a. Very bad! (Industrial area/lots of smog) (−5)

The good news is that cities are cleaner now than they were even ten years ago. The bad news is that you are still exposed to significant air pollution. If you are experiencing new respiratory symptoms and you don't smoke, consider air pollution as a potential cause and discuss this with your physician or a specialist. Even if you must work in the polluted city, give serious thought to moving somewhere removed from all that pollution.

b. Average! (Urban area/medium smog) (0)

The good news is that cities are cleaner now than they were even ten years ago. The bad news is that you are still exposed to air pollution. If you are experiencing new respiratory symptoms and you don't smoke, consider air pollution as a potential cause and discuss this with your physician or a specialist. Don't go out exerting yourself when smog alerts are in effect.
c. Okay! (Suburbs/low smog) (+1)

The good news is that cities are cleaner now than they were even ten years ago. The bad news is that you are still exposed to air pollution. If you are experiencing new respiratory symptoms and you don't smoke, consider air pollution as a potential cause and discuss this with your physician or a specialist. Don't go out exerting yourself when smog alerts are in effect.

d. Very good! (Country side/no smog) (+2) Ahhh. Take a deep breath of that clean fresh air! It is wonderful and life expectancy-enhancing that you live in a smog-free environment.
2. Seat Belts

a. Always (+2)

You obviously know the statistics about survival from a car crash with a seat belt versus without one. Continue to be diligent in wearing your seat belt.
Wearing a seatbelt, even in the presence of an airbag, dramatically increases your chances of minimizing injury or surviving a serious car accident.

b. About 80% of the time (0)

Good job with wearing your seatbelt most of the time. Many people don't put their seat belts on because they assume nothing will happen if it's just a short drive. The statistics clearly tell a different story. When you feel it is too much of a bother to put on your seatbelt, really consider how much effort it really takes to snap that belt in. Then consider how much effort it would take you to recover from a concussion or even worse.
Wearing a seatbelt, even in the presence of an airbag, dramatically increases your chances of minimizing injury or surviving a serious car accident.

c. Half of the time (−5)

The majority of lethal car accidents occur within 5 miles of the driver's home. Many people don't put their seat belts on because they assume nothing will happen if it's just a short drive. The statistics clearly tell a different story. Many lethal accidents, including those that involve deployed air bags, would not be lethal if the person had been wearing their seat belts.
Wearing a seatbelt, even in the presence of an airbag, dramatically increases your chances of minimizing injury or surviving a serious car accident.
d. Less than half of the time (−7)

The majority of lethal car accidents occur within 5 miles of the driver's home. Many people don't put their seat belts on because they assume nothing will happen if it's just a short drive. The statistics clearly tell a different story. Many lethal accidents, including those that involve deployed air bags, would not be lethal if the person had been wearing their seat belts.
Wearing a seatbelt, even in the presence of an airbag, dramatically increases your chances of minimizing injury or surviving a serious car accident.
3. Coffee
a. None (0)
   Whatever your reason for not drinking coffee, it definitely has you on the right track. Don't start because it is much harder to stop the habit once you pick it up.

Excessive coffee can be a sign of increased stress. Stress can lead to a hormonal imbalance, which can physically stress and age numerous organs. In addition, coffee predisposes the stomach to chronic inflammation of the stomach and ulcers. Such chronic inflammation leads to release of substances that raise the risk of heart disease. Tea, and especially green tea, on the other hand, has been noted for its significant antioxidant content, and tea drinkers in general appear to be healthier.

b. 1 to 2 cups (−1)
One to two cups of coffee are certainly better than drinking more than that. You are doing a good job keeping your coffee consumption down to a minimum. If you can now take those 1-2 cups of coffee and convert them to tea, even better!

Excessive coffee can be a sign of increased stress. Stress can lead to a hormonal imbalance, which can physically stress and age numerous organs. In addition, coffee predisposes the stomach to chronic inflammation of the stomach and ulcers. Such chronic inflammation leads to release of substances that raise the risk of heart disease. Tea, and especially green tea, on the other hand, has been noted for its significant antioxidant content, and tea drinkers in general appear to be healthier.

c. Over 3 cups (−3)
You are drinking too much coffee. You may have many reasons for this degree of consumption including trouble with sleep and staying awake, withdrawal headaches, the taste, and so on. Gradually replacing your coffee with tea is one method of weaning yourself off. There are effective methods for the majority of people and the effort is worthwhile in improving quality and quantity of life.

Excessive coffee can be a sign of increased stress. Stress can lead to a hormonal imbalance, which can physically stress and age numerous organs. In addition, coffee predisposes the stomach to chronic inflammation of the stomach and ulcers. Such chronic inflammation leads to release of substances that raise the risk of heart disease. Tea, and especially green tea, on the other hand, has been noted for its significant antioxidant content, and tea drinkers in general appear to be healthier.
4. Tea

a. None (0) Try to give tea a chance. You may find that it grows on you. Regular tea consumption is a healthy habit that may actually be life extending. The antioxidants in tea may decrease your risk of heart disease and cancer.
Tea contains a powerful class of antioxidants known as polyphenols. It is controversial whether green tea has more bioavailable polyphenols than black tea. Either way however, you can't go wrong.

b. 1-2 cups per day (+2) Good job on the tea drinking! Regular tea consumption is a healthy habit that may actually be life extending. The antioxidants in tea may decrease your risk of heart disease and cancer.
Tea contains a powerful class of antioxidants known as polyphenols. It is controversial whether green tea has more bioavailable polyphenols than black tea. Either way however, you can't go wrong.

c. 3-5 cups per day (+5) You are a tea connoisseur! This is a very healthy habit and because of the antioxidants in tea, it may well be life extending as well.
Tea contains a powerful class of antioxidants known as polyphenols. It is controversial whether green tea has more bioavailable polyphenols than black tea. Either way however, you can't go wrong.

d. 6-10 cups per day (−3) While tea is generally good for you, 6-10 cups a day seems like a bit much. The concern isn't so much that this much tea is bad for you, bit rather, all that tea drinking might be a marker of stress. If you back down on the things that are stressful for you, then your tea (and coffee?) consumption might come down as well.

e. Greater than 10 cups per day (−6) While tea is generally good for you, more than 10 cups a day seems like a bit much. The concern isn't so much that this much tea is bad for you, bit rather, all that tea drinking might be a marker of stress. If you back down on the things that are stressful for you, then your tea (and coffee?) consumption might come down as well.
5a. Do you smoke or are you exposed to second-hand smoke? [Do Not Provide a heading or feedback for this question]
a. Yes (0)*
b. No (0)*
   *[We don't need a score for the above . . . use the below scores for smoking. It is a good question to set the stage.]
5a-c. If your answer was YES, what is your daily tobacco exposure (cigarettes, cigars, pipe, chewing tobacco)? THE FEEDBACK HERE IS FOR A COMBINATION OF QUESTIONS 5a-c AND THE SCORE IS THE PRODUCT OF 5b and 5c. [Heading should be: “Smoking”]

a. Score: −216 to −48
Of all the issues that you can address as a result of taking this healthspan calculator, this is the one to tackle! Nothing, short of extreme obesity, will shorten your health span more and cause you to have a long period of chronic illness and decreased quality of life than your smoking habit. You are exposing yourself to a prevalent and important cause of people dying in their 70s and earlier. 400,000 deaths per year are attributed to smoking and many more people suffer from smoking-related and debilitating diseases each year. Please try to stop smoking for yourself, your family and your friends. You will be helping society by cutting down on the high cost of caring for people with smoking-related illnesses . . . perhaps the highest single health-related expense in this country today.
Cigarette smoke contains toxins, which directly damage DNA and subsequently cause cancer. Cigarettes are the biggest direct source of nitro amines humans are exposed to. These substances along with other constituents of cigarette smoke are potent oxidants and carcinogens that lead to accelerated aging, and diseases associated with aging. Each day, nearly 5,000 adolescents (aged 11-17) smoke their first cigarette. Almost two million teens annually, and approximately one-third of those that become smokers will eventually die of smoking-related illnesses. Helpful internet sites: Quitnet [http://www.quitnet.org/qn_main.jtml] and the American Lung Association [http://www.lungusa.org/]

b. Score −47 to −36
Of all the issues that you can address as a result of taking this healthspan calculator, this is the one to tackle! Nothing, short of extreme obesity, will shorten your health span more and cause you to have a long period of chronic illness and decreased quality of life than your smoking habit. You are exposing yourself to a prevalent and important cause of people dying in their 70s and earlier. 400,000 deaths per year are attributed to smoking and many more people suffer from smoking-related and debilitating diseases each year. Please try to stop smoking for yourself, your family and your friends. You will be helping society by cutting down on the high cost of caring for people with smoking-related illnesses . . . perhaps the highest single health-related expense in this country today.
Cigarette smoke contains toxins, which directly damage DNA and subsequently cause cancer. Cigarettes are the biggest direct source of nitro amines humans are exposed to. These substances along with other constituents of cigarette smoke are potent oxidants and carcinogens that lead to accelerated aging, and diseases associated with aging. Each day, nearly 5,000 adolescents (aged 11-17) smoke their first cigarette. Almost two million teens annually, and approximately one-third of those that become smokers will eventually die of smoking-related illnesses. Helpful internet sites: Quitnet [http://www.quitnet.org/qn_main.jtml] and the American Lung Association [http://www.lungusa.org/]

c. Score −36 to −6
Any smoking is harmful and shortens your life. Those who smoke sometimes or often are more likely to expose themselves to second hand smoke as well which is even more toxic. You are exposing yourself to a prevalent and important cause of people dying in their 70s and earlier. 400,000 deaths per year are attributed to smoking and many more people suffer from smoking-related and debilitating diseases each year. Please try to stop smoking for yourself, your family and your friends. You will be helping society by cutting down on the high cost of caring for people with smoking-related illnesses . . . perhaps the highest single health-related expense in this country today.
Cigarette smoke contains toxins, which directly damage DNA and subsequently cause cancer. Cigarettes are the biggest direct source of nitro amines humans are exposed to. These substances along with other constituents of cigarette smoke are potent oxidants and carcinogens that lead to accelerated aging, and diseases associated with aging. Each day, nearly 5,000 adolescents (aged 11-17) smoke their first cigarette. Almost two million teens annually, and approximately one-third of those that become smokers will eventually die of smoking-related illnesses. Helpful internet sites: Quitnet [http://www.quitnet.org/qn_main.jtml] and the American Lung Association [http://www.lungusa.org/]

d. Score 0-5
By not smoking or being exposed to substantial second hand smoke, you are avoiding a prevalent and important cause of people dying in their 70s and earlier. 400,000 deaths per year are attributed to smoking and many more people suffer from smoking-related and debilitating diseases each year.
Cigarette smoke contains toxins, which directly damage DNA and subsequently cause cancer. Cigarettes are the biggest direct source of nitro amines humans are exposed to. These substances along with other constituents of cigarette smoke are potent oxidants and carcinogens that lead to accelerated aging, and diseases associated with aging. Each day, nearly 5,000 adolescents (aged 11-17) smoke their first cigarette. Almost two million teens annually, and approximately one-third of those that become smokers will eventually die of smoking-related illnesses. Helpful internet sites: Quitnet [http://www.quitnet.org/qn_main.jtml] and the American Lung Association [http://www.lungusa.org/]
   5D. SECOND-HAND SMOKE

a. Daily (−5)

Do everything you can to end your exposure to tobacco smoke. If it is a loved one or someone else that you live with, encourage them to stop smoking, if not for them, then for their family and their friends. Avoiding second hand smoke is a very important habit. Because second hand smoke is even more toxic than the filtered smoke that smokers inhale, it takes less of a “dose” or exposure to be toxic to your lungs and your body in general. Do what you can to stop exposing yourself to this very harmful smoke.
Second hand smoke is more toxic than what the smoker gets because it is unfiltered. Such exposure is clearly a substantial cause of cancer, heart disease, asthma and other lung diseases.

b. Not daily, but often (−2)

Do everything you can to end your exposure to tobacco smoke. If it is a loved one or someone else that you live with, encourage them to stop smoking, if not for them, then for their family and their friends. Avoiding second hand smoke is a very important habit. Because second hand smoke is even more toxic than the filtered smoke that smokers inhale, it takes less of a “dose” or exposure to be toxic to your lungs and your body in general. Do what you can to stop exposing yourself to this very harmful smoke.
Second hand smoke is more toxic than what the smoker gets because it is unfiltered. Such exposure is clearly a substantial cause of cancer, heart disease, asthma and other lung diseases.

c. Sometimes (−1)

Avoiding second hand smoke is a very important habit. Because second hand smoke is even more toxic than the filtered smoke that smokers inhale, it takes less of a “dose” or exposure to be toxic to your lungs and your body in general. Do what you can to stop exposing yourself to this very harmful smoke. Second hand smoke is more toxic than what the smoker gets because it is unfiltered. Such exposure is clearly a substantial cause of cancer, heart disease, asthma and other lung diseases.

d. Rarely or never (0)

Avoiding second hand smoke is a very important habit. Because second hand smoke is even more toxic than the filtered smoke that smokers inhale, it takes less of a “dose” or exposure to be toxic to your lungs and your body in general. Thus, keep avoiding the secondhand smoke as you have been doing.
Second hand smoke is more toxic than what the smoker gets because it is unfiltered. Such exposure is clearly a substantial cause of cancer, heart disease, asthma and other lung diseases.
5e. Past History of Smoking
(a) I did not smoke in the past (0)
No comment needed [IF THEIR ANSWER WAS THIS OPTION, THEN DO NOT PROVIDE THIS HEADING OR ANY FEEDBACK HERE]
(b) I have chronic lung disease from my past history of smoking (for example, resulting in shortness of breath with minimal exertion, requiring supplemental oxygen, or a diagnosis of emphysema or chronic obstructive lung disease (COPD) (−12)

It is difficult to gauge how much less your risk of vascular disease (stroke, heart attack) or cancer is once you are five years away from smoking compared to if you continued to smoke. The fact that you have sustained at least some damage from smoking would unfortunately indicate that you are likely at some increased risk compared to if you had never smoked. There are a number of intangibles that make it difficult to further estimate this risk. At the least, be extra diligent with your health care provider in screening for other smoking-related illnesses.
(c) I quite and seem to have minimal or no lung problems (0)

Thank goodness you quit smoking before you sustained functionally significant lung disease. It is difficult to gauge how much less your risk of vascular disease (stroke, heart attack) or cancer is once you are five years away from smoking compared to if you continued to smoke. There are a number of intangibles that make it difficult to further estimate this risk.

At the least, be extra diligent with your health care provider in screening for smoking-related illnesses.
6.a) HEADING FOR THE COMBINATION OF 6a and b: “Alcohol”
a. I don't drink alcohol (0)
b. 1 or 2 days per week (1)
c. 3 to 5 days per week (3)
d. Every day of the week (5)
6.b) On days when you drink alcoholic beverages (beer, wine and mixed drinks) how many glasses do you usually drink?
a. I don't drink (0)
b. 1-2 (+2)
c. 3-4 (−5)
d. 5 or more (−10)
   [For this scoring, please multiply the score from 6a with the score in 6b.]:

a. No alcohol (sum=0)
There is substantial evidence inferring that mild to moderate alcohol consumption can be good for you. Some people cannot tolerate even a small amount of alcohol for medical or other reasons. If you like the idea of a glass of wine or beer with dinner, know that it may be good for you as well. Moderate alcohol consumption has been associated with decreased heart disease risk. This may be one explanation for the “French paradox”, in which the French are known for their love of high saturated fat foods, and yet their heart disease risks may be lower (except in the case of those who smoke cigarettes), perhaps because of the higher consumption of wine in that country. Significant controversy revolves around what type of alcohol (wine, beer or liquor) helps.
Remember that on the other hand, excessive alcohol is a toxin, which damages the liver and the mitochondria within most cells of the body. This leads to acceleration of aging and increased susceptibility to many diseases associated with aging.

b. Product=+1 to +10)
A moderate amount of alcohol consumption as you have indicated may be healthy for you and possibly reduces the risk of cardiovascular disease and stroke. There is substantial evidence inferring that mild to moderate alcohol consumption can be good for you. One study suggests that even a little more frequent consumption (one glass a day) might be good for you. However, some people cannot tolerate even a small amount of alcohol for medical or other reasons. Discuss your intake with your health care provider.
Moderate alcohol consumption has been associated with decreased heart disease risk. This may be one explanation for the “French paradox”, in which the French are known for their love of high saturated fat foods, and yet their heart disease risks may be lower (except in the case of those who smoke cigarettes), perhaps because of the higher consumption of wine in that country. Significant controversy revolves around what type of alcohol (wine, beer or liquor) helps.
Remember that on the other hand, excessive alcohol is a toxin, which damages the liver and the mitochondria within most cells of the body. This leads to acceleration of aging and increased susceptibility to many diseases associated with aging.

c. Product=−1 to −15
Moderate alcohol consumption has been associated with decreased heart disease risk. This may be one explanation for the “French paradox”, in which the French are known for their love of high saturated fat foods, and yet their heart disease risks may be lower (except in the case of those who smoke cigarettes), perhaps because of the higher consumption of wine in that country. Significant controversy revolves around what type of alcohol (wine, beer or liquor) helps.
However your answer regarding alcohol consumption suggests that you consume too much alcohol. Excessive alcohol is a toxin, which damages the liver and the mitochondria within most cells of the body. This leads to acceleration of aging and increased susceptibility to many diseases associated with aging. More information: http://www.niaaa.nih.gov/publications/booklet.htm

d. Product=−16 to −30

Moderate alcohol consumption has been associated with decreased heart disease risk. This may be one explanation for the “French paradox”, in which the French are known for their love of high saturated fat foods, and yet their heart disease risks may be lower (except in the case of those who smoke cigarettes), perhaps because of the higher consumption of wine in that country. Significant controversy revolves around what type of alcohol (wine, beer or liquor) helps.
However your answer regarding alcohol consumption suggests that you consume too much alcohol. Excessive alcohol is a toxin, which damages the liver and the mitochondria within most cells of the body. This leads to acceleration of aging and increased susceptibility to many diseases associated with aging.

Alcohol, consumed in the amount that you have indicated, can be toxic to your liver and your nervous system. For some amounts such as this could mean you are addicted to alcohol. Ask yourself the following 4 questions, known as the CAGE questions:

1. Have you tried to CUT down in your drinking?

2. Are you ANNOYED with criticisms about drinking?

3. Do you sometimes feel GUILTY about drinking?

4. Do you sometimes drink alcohol in the morning or as an “EYE-OPENER”?

If you answer “yes” to at least 2 of these questions, there is a 75% chance that you are addicted to alcohol. On the other hand, if you answer yes to just one question or to none of them, there is a 96% chance that you are not. Having a problem with alcohol will shorten your health span and will certainly decrease your quality of life. Seeking assistance and treatment is critical.
Excessive alcohol is a toxin, which damages the liver and the mitochondria within most cells of the body. This leads to acceleration of aging and increased susceptibility to many diseases associated with aging. Moderate alcohol consumption has been associated with decreased heart disease risk. This may be one explanation for the “French paradox”, in which the French are known for their love of high saturated fat foods, and yet their heart disease risks may be lower (except in the case of those who smoke cigarettes), perhaps because of the higher consumption of wine in that country. More information: http://www.niaaa.nih.gov/publications/booklet.htm

e. Product=−31 to −50 Alcohol, consumed in the amount that you have indicated, is toxic to your liver and your nervous system. You are at significant risk of developing liver disease and eventually liver failure. Consumption in the amounts you have indicated likely indicated you are addicted to alcohol. Ask yourself the following 4 questions, known as the CAGE questions:

1. Have you tried to CUT down in your drinking?

2. Are you ANNOYED with criticisms about drinking?

3. Do you sometimes feel GUILTY about drinking?

4. Do you sometimes drink alcohol in the morning or as an “EYE-OPENER”?

If you answer “yes” to at least 2 of these questions, there is a 75% chance that you are addicted to alcohol. On the other hand, if you answer yes to just one question or to none of them, there is a 96% chance that you are not. Having a problem with alcohol will shorten your health span and will certainly decrease your quality of life. Seeking assistance and treatment is critical.
Excessive alcohol is a toxin, which damages the liver and the mitochondria within most cells of the body. This leads to acceleration of aging and increased susceptibility to many diseases associated with aging. Moderate alcohol consumption has been associated with decreased heart disease risk. This may be one explanation for the “French paradox”, in which the French are known for their love of high saturated fat foods, and yet their heart disease risks may be lower (except in the case of those who smoke cigarettes), perhaps because of the higher consumption of wine in that country. More information: http://www.niaaa.nih.gov/publications/booklet.htm
7. Aspirin
a. Never (0)
   Perhaps you cannot tolerate an aspirin a day because it hurts your stomach or you have a propensity for bleeding. On the other hand, if you really don't have a reason to not take an aspirin a day, consider taking one daily.
   81 mg of Aspirin per day has been noted to significantly decrease heart disease risk. This benefit may be due to the anti-blood clotting effects of aspirin. Chronic inflammation may also play a role in heart disease (see 11, below) and therefore, aspirin's effect on inflammation may also be helpful.
      For more information go to: American Heart Association's findings [http://www.americanheart.org/]

b. Occasionally (0)

Taking an aspirin daily has been shown to decrease the risk of heart attack by half. Try to increase your intake to daily.
81 mg of Aspirin per day has been noted to significantly decrease heart disease risk. This benefit may be due to the anti-blood clotting effects of aspirin. Chronic inflammation may also play a role in heart disease (see 11, below) and therefore, aspirin's effect on inflammation may also be helpful.
   For more information go to: American Heart Association's findings [http://www.americanheart.org/]

c. Frequently (+2)

Good for you! An aspirin a day has been shown, particularly among men, to cut the risk of heart attack in half.
81 mg of Aspirin per day has been noted to significantly decrease heart disease risk. This benefit may be due to the anti-blood clotting effects of aspirin. Chronic inflammation may also play a role in heart disease (see 11, below) and therefore, aspirin's effect on inflammation may also be helpful.
   For more information go to: American Heart Association's findings [http://www.americanheart.org/]

d. Every day (+10)

Good for you! An aspirin a day has been shown, particularly among men, to cut the risk of heart attack in half.
81 mg of Aspirin per day has been noted to significantly decrease heart disease risk. This benefit may be due to the anti-blood clotting effects of aspirin. Chronic inflammation may also play a role in heart disease (see 11, below) and therefore, aspirin's effect on inflammation may also be helpful.
   For more information go to: American Heart Association's findings [http://www.americanheart.org/]
8. Sunscreen

a. Rarely or never (−3)

You are doing an poor job of protecting yourself from the sun and therefore from accelerated aging of your skin as well as from deadly skin cancers such as melanoma. But you could do better. The payoff could be big! Especially when you are not as diligent as you should be in protecting your skin, you should have a regular skin (dermatology) check up and perform a monthly self-examination of your skin.
The association between sun exposure and accelerated skin aging are clear. The ultraviolet rays in sunlight directly damages DNA. More sun means more wrinkles sooner. It also means a higher risk of deadly skin cancer. Excessive sun exposure may also have toxic consequences for the body in general. For guidance on a self-exam, see skincheck.com [http://www.skincheck.com/]

b. Sometimes (−1)

You are doing an ok job of protecting yourself from the sun and therefore from accelerated aging of your skin as well as from deadly skin cancers such as melanoma. But you could do better. The payoff could be big! Especially when you are not as diligent as you should be in protecting your skin, you should have a regular skin (dermatology) check up and perform a monthly self-examination of your skin.
The association between sun exposure and accelerated skin aging are clear. The ultraviolet rays in sunlight directly damages DNA. More sun means more wrinkles sooner. It also means a higher risk of deadly skin cancer. Excessive sun exposure may also have toxic consequences for the body in general. For guidance on a self-exam, see skincheck.com [http://www.skincheck.com/]

c. Most of the time (+1)

You are doing a good job protecting yourself from the sun and therefore from accelerated aging of your skin as well as deadly skin cancers such as from melanoma. Just because you do protect yourself, does not mean you should not have a regular skin (dermatology) check up and perform a monthly self-examination of your skin.
The association between sun exposure and accelerated skin aging are clear. The ultraviolet rays in sunlight directly damages DNA. More sun means more wrinkles sooner. It also means a higher risk of deadly skin cancer. Excessive sun exposure may also have toxic consequences for the body in general. For guidance on a self-exam, see skincheck.com [http://www.skincheck.com/]

d. Always (+3)

You are doing a good job protecting yourself from the sun and therefore from accelerated aging of your skin as well as deadly skin cancers such as from melanoma. Just because you do protect yourself, does not mean you should not have a regular skin (dermatology) check up and perform a monthly self-examination of your skin.

The association between sun exposure and accelerated skin aging are clear. The ultraviolet rays in sunlight directly damages DNA. More sun means more wrinkles sooner. It also means a higher risk of deadly skin cancer. Excessive sun exposure may also have toxic consequences for the body in general. For guidance on a self-exam, see skincheck.com [http://www.skincheck.com/]
9. Sex and Drugs

a. Never (0)

You are exercising good judgment.
Viruses such as HIV and others, which are transmitted by risky behavior not only cause AIDS but also various cancers including lymphoma. AIDS and Hepatitits of course can be lethal. These viruses can also change DNA and probably also, as a result, influence aging as well. For more information, go to: http://www.advocatesforyouth.org/youth/index.htm

b. Rarely (once a year or less) (−3)

Be careful! Assumptions about the infectious status of your partner or that needle might end up being a deadly assumption. It is much wiser to be safe and not sorry.
Viruses such as HIV and others, which are transmitted by risky behavior not only cause AIDS but also various cancers including lymphoma. AIDS and Hepatitits of course can be lethal. These viruses can also change DNA and probably also, as a result, influence aging as well. For more information, go to: http://www.advocatesforyouth.org/youth/index.htm

c. Sometimes (few times a year) (−6)

Be careful! Assumptions about the infectious status of your partner or that needle might end up being a deadly assumption. It is much wiser to be safe and not sorry. Viruses such as HIV and others, which are transmitted by risky behavior not only cause AIDS but also various cancers including lymphoma. AIDS and Hepatitits of course can be lethal. These viruses can also change DNA and probably also, as a result, influence aging as well. For more information, go to: http://www.advocatesforyouth.org/youth/index.htm
d. Often (every few months (−12)
Your sex and/or drug related behavior is critically putting your life and others' lives at risk.
   Viruses such as HIV and others, which are transmitted by risky behavior not only cause AIDS but also various cancers including lymphoma. AIDS and Hepatitits of course can be lethal. These viruses can also change DNA and probably also, as a result, influence aging as well. For more information, go to: http://www.advocatesforyouth.org/youth/index.htm
d. Very often (once or more a month) (−18)
Your sex and/or drug related behavior is critically putting your life and other's lives at risk.
   Viruses such as HIV and others, which are transmitted by risky behavior not only cause AIDS but also various cancers including lymphoma. AIDS and Hepatitits of course can be lethal. These viruses can also change DNA and probably also, as a result, influence aging as well. For more information, go to: http://www.advocatesforyouth.org/youth/index.htm
10. Flossing

a. Yes (+5)

Keep flossing away! Not only will it improve the chances of longer kisses, but it improves your heart health as well.
Recent scientific evidence reveals that chronic gum disease leads to the release of inflammatory, toxic substances and certain bacteria into the blood stream which potentiate plaque formation in arteries and ultimately lead to heart disease. This process probably also increases the risk of stroke and accelerated aging. For more information, go to: American Dental Association's findings [http://www.ada.org/].
B. NO (−7)

YOU NEED TO FLOSS YOUR TEETH. THERE ARE BENEFITS THAT GO FAR BEYOND BETTER BREATH. BUT DON'T UNDERESTIMATE THE BENEFITS OF GOOD BREATH! DILIGENT AND REGULAR FLOSSING MEANS KEEPING YOUR TEETH AND VERY POSSIBLY REDUCING YOUR RISK OF HEART ATTACK.
Recent scientific evidence reveals that chronic gum disease leads to the release of inflammatory, toxic substances and certain bacteria into the blood stream which potentiate plaque formation in arteries and ultimately lead to heart disease. This process probably also increases the risk of stroke and accelerated aging. For more information, go to: American Dental Association's findings [http://www.ada.org/].

c. I have dentures (0)

[No need to provide feedback] {IF THEY ANSWER THIS OPTION DO NOT PROVIDE HEADING OR FEEDBACK FOR THIS QUESTION}
C. Nutrition & Exercise

Your Weight

1a, b, c. Calculation of body mass index.

First, if the respondent answers “a. yes” to question c: Do you body build or strength train to the degree that your. body mass index is high because of muscle, not because of fat?, then provide the following feedback, and DO NOT provide feedback according to their calculated BMI.

(a) yes (+10)

Excellent! Exercise in general, and specifically building muscle (but not at the expense of hurting your joints) is so good for you. Muscle is an exceptionally efficient burner of fat, particularly the fat that collects in the muscles themselves (called intramuscular fat). Getting rid of fat, particularly the type that collects in your muscles and in your belly (called visceral fat) is key to preventing or delaying age-related diseases. Fat produces cytokines and other harmful substances that directly lead to blockages in the blood vessels that feed your heart, brain and other vital organs (which in turn means a greater likelihood of heart attack, stroke, dementia and most other problems that can become more prevalent at older ages). So, if you feel you are lean, with very little fat, fantastic! If on the other hand you still have a significant amount of fat to lose, to be honest, you should take a few years off the helathspan that was calculated for you (sorry about that!).
According to Harvard's Mr. Fit Study, building muscle is possible at any age, even among people who are at first very frail and who are very old. And, the benefits of building the muscle back up are especially evident and rapid in these cases. The benefits are surprising beyond the expected, such as improved balance, gait and decreased risk of falls; they also include improved sleep and cognitive function! In other words, muscle building that leads to a lean body mass is likely the most important single intervention a person can do to slow down their aging and delay or perhaps even escape age-related illnesses.

If respondent answers “(b) no” (no body building), then provide feedback according to the calculated BMI.

Provide feedback according to the following calculated BMIs . . .

BMI <17, (−5).

You may be too thin. Some people who are this thin have significant problems with their health, particularly if this is related to an eating disorder like anorexia or bulimia or because they for some other reason don't have an adequate diet. On the other hand, you may be very healthy . . . this is actually a tough call for the calculator and brings up the issue that tools such as these are pretty good at giving general advice, but there are always people who are exceptions to the rule. Thus, if you feel healthy at this weight, great and when you should add a few years to the healthspan that was calculated for you (sorry about the miscalculation!). If on the other hand, your weight loss is relatively new and unexplained, you should talk with your doctor about it.

BMI 17 to <25 (+10)

Excellent! You are a lean, mean fighting machine! Maintaining a lean body mass is one of the most important things you can do to maximize your healthspan. Keep doing what you are doing.
As you age, you may find it more challenging to keep the weight off. You will find, if you aren't doing it already, that getting into a regular regimen of weight training to maintain or build muscle mass will be very effective in keeping the weight off. Of course diet is also important and lowering your consumption of carbohydrates is helpful. Diet advice is covered by the questions related to carbohydrates and sweets.

BMI 25 to <26 (−5)

Your calculated body mass index would indicate that you are likely a bit overweight. If you are in fact lean and this calculation is off because of your unique build and greater than expected amount of muscle, then this assumption is incorrect and you should add a few years to your calculated healthspan (with apologies). However, if, to be honest you are a bit overweight, then you should do what you can to get down to a lean (as little fat as possible) body weight. Even being just a bit overweight significantly increases your risk for diseases that markedly impact upon your healthspan including heart trouble, diabetes, high blood pressure, cancer, stroke and dementia. So, you are almost at a good weight. Please set aside the 30 minutes a day to exercise and be on a diet that will lead to your losing some weight (burn more calories than you take in) and eventually maintaining a healthspan maximizing weight.
As you age, you may find it more challenging to keep the weight off. You will find, if you aren't doing it already, that getting into a regular regimen of weight training to maintain or build muscle mass will be very effective in keeping the weight off. Of course diet is also important and lowering your consumption of carbohydrates is helpful. Diet advice is covered by the questions related to carbohydrates and sweets.

BMI 26 to <29 (−7)

Your calculated body mass index indicates that you are likely overweight. If you are in fact lean (minimal amount of fat) and this calculation is off because of your unique build and greater than expected amount of muscle, then this assumption is incorrect and you should add a few years to your calculated healthspan (with apologies). Though, if this is the case, perhaps you should have answered “yes” the bodybuilding/strength training question. If, to be honest, you are a bit overweight, then you should do what you can to get down to a lean (as little fat as possible) body weight. Being overweight significantly increases your risk for diseases that markedly impact upon your healthspan including heart trouble, diabetes, high blood pressure, cancer, stroke and dementia. Set aside the 30 minutes a day to exercise and be on a diet that will lead to your losing some weight (burn more calories than you take in) and eventually maintaining a healthspan maximizing weight.
As you age, you may find it even more challenging to keep the weight off. You will find, if you aren't doing it already, that getting into a regular regimen of weight training to maintain or build muscle mass will be very effective in keeping the weight off. Of course diet is also important and lowering your consumption of carbohydrates is helpful. Diet advice is covered by the questions related to carbohydrates and sweets.

BMI 29 to <34 (−10)

Your calculated body mass index indicates that you are significantly overweight. If you are in fact lean (minimal amount of fat) and this calculation is off because of your unique build and greater than expected amount of muscle, perhaps it would have been better to answer yes to the body building/strength training question (and you should add a few years to your calculated healthspan).
If, to be honest, you are overweight, then you should do what you can to get down to a lean (as little fat as possible) body weight. Being overweight significantly increases your risk for diseases that markedly impact upon your healthspan including heart trouble, diabetes, high blood pressure, cancer, stroke and dementia. Set aside the 30 minutes a day to exercise and be on a diet that will lead to your losing some weight (burn more calories than you take in) and eventually maintaining a healthspan maximizing weight.
As you age, you may find it even more challenging to keep the weight off. You will find, if you aren't doing it already, that getting into a regular regimen of weight training to maintain or build muscle mass will be very effective in keeping the weight off. Of course diet is also important and lowering your consumption of carbohydrates is helpful. Diet advice is covered by the questions related to carbohydrates and sweets.

BMI 34 to <39 (−24)

Your calculated body mass index indicates that you are significantly overweight. If you are in fact lean (minimal amount of fat) and this calculation is off because of your unique build and greater than expected amount of muscle, perhaps it would have been better to answer yes to the body building/strength training question (and you should add a few years to your calculated healthspan).
If, to be honest, you are overweight, then you should do what you can to get down to a lean (as little fat as possible) body weight. Being overweight significantly increases your risk for diseases that markedly impact upon your healthspan including heart trouble, diabetes, high blood pressure, cancer, stroke and dementia. Set aside the 30 minutes a day to exercise and be on a diet that will lead to your losing some weight (burn more calories than you take in) and eventually maintaining a healthspan maximizing weight.
As you age, you may find it even more challenging to keep the weight off. You will find, if you aren't doing it already, that getting into a regular regimen of weight training to maintain or build muscle mass will be very effective in keeping the weight off. Of course diet is also important and lowering your consumption of carbohydrates is helpful. Diet advice is covered by the questions related to carbohydrates and sweets.

BMI >39 (−60)

Your calculated body mass index indicates that you are significantly overweight. If you are in fact lean (minimal amount of fat) and this calculation is off because of your unique build and greater than expected amount of muscle, perhaps it would have been better to answer yes to the body building/strength training question (and you should add a few years to your calculated healthspan).
If, to be honest, you are overweight, then you should do what you can to get down to a lean (as little fat as possible) body weight. Being overweight significantly increases your risk for diseases that markedly impact upon your healthspan including heart trouble, diabetes, high blood pressure, cancer, stroke and dementia. Set aside the 30 minutes a day to exercise and be on a diet that will lead to your losing some weight (burn more calories than you take in) and eventually maintaining a healthspan maximizing weight.
As you age, you may find it even more challenging to keep the weight off. You will find, if you aren't doing it already, that getting into a regular regimen of weight training to maintain or build muscle mass will be very effective in keeping the weight off. Of course diet is also important and lowering your consumption of carbohydrates is helpful. Diet advice is covered by the questions related to carbohydrates and sweets.

2a. Fast Food

a. None (0)

Your answer suggests that you avoid fast food restaurants and preserved meats. You are already going a long way towards a healthy diet. Keep it up!
Fast food, generally fried foods and hamburgers, are high in calories and saturated fats. These will make you gain weight and they increase your risk for heart disease, stroke and perhaps cancer. Some studies suggest that 90% of all human cancers are environmentally induced, 30-40% of these by diet. Preserved and cured meats (bacon, sausage, lunch meats, etc.) are the largest source of nitrites in our diet. Nitrites lead to the formation in our bodies of nitrosoamines, which are important environmental oxidants and probable carcinogens. For instance, there is a suggestive association between nitrosamines and stomach cancer.

b. 1-2 servings (−1)

Fast food, generally fried foods and hamburgers, are high in calories and saturated fats. These will make you gain weight and they increase your risk for heart disease, stroke and perhaps cancer. Another potential risk factor for cancer is preserved meats.
Fast food, generally fried foods and hamburgers, are high in calories and saturated fats. These will make you gain weight and they increase your risk for heart disease, stroke and perhaps cancer. Some studies suggest that 90% of all human cancers are environmentally induced, 30-40% of these by diet. Preserved and cured meats (bacon, sausage, lunch meats, etc.) are the largest source of nitrites in our diet. Nitrites lead to the formation in our bodies of nitrosoamines, which are important environmental oxidants and probable carcinogens. For instance, there is a suggestive association between nitrosamines and stomach cancer.

c. 3-4 servings (−3)

You go to the fast food restaurants way too much. Please consider what a weekly intake of this unhealthy diet will do to you over the course of a year. Fast food, generally fried foods and hamburgers, are high in calories and saturated fats. These will make you gain weight and they increase your risk for heart disease, stroke and perhaps cancer. Another potential risk factor for cancer is preserved meats. Do whatever you can to make your diet more healthy.
Fast food, generally fried foods and hamburgers, are high in calories and saturated fats. These will make you gain weight and they increase your risk for heart disease, stroke and perhaps cancer. Some studies suggest that 90% of all human cancers are environmentally induced, 30-40% of these by diet. Preserved and cured meats (bacon, sausage, lunch meats, etc.) are the largest source of nitrites in our diet. Nitrites lead to the formation in our bodies of nitrosoamines, which are important environmental oxidants and probable carcinogens. For instance, there is a suggestive association between nitrosamines and stomach cancer.

d. Over 5 servings (−8)

You go to the fast food restaurants way too much. Please consider what a weekly intake of this unhealthy diet will do to you over the course of a year. Fast food, generally fried foods and hamburgers, are high in calories and saturated fats. These will make you gain weight and they increase your risk for heart disease, stroke and perhaps cancer. Another potential risk factor for cancer is preserved meats. Do whatever you can to make your diet more healthy.
Fast food, generally fried foods and hamburgers, are high in calories and saturated fats. These will make you gain weight and they increase your risk for heart disease, stroke and perhaps cancer. Some studies suggest that 90% of all human cancers are environmentally induced, 30-40% of these by diet. Preserved and cured meats (bacon, sausage, lunch meats, etc.) are the largest source of nitrites in our diet. Nitrites lead to the formation in our bodies of nitrosoamines, which are important environmental oxidants and probable carcinogens. For instance, there is a suggestive association between nitrosamines and stomach cancer.
2b. Grilling Food

a. I am a vegetarian. (0)*

It is great that you are a vegetarian. That is fantastic news. Clearly, such a diet will add healthy years to your life.

b. I never barbecue. (0)

Not barbecuing your food helps you avoid the carcinogens that can develop as a result of cooking meat and fish at very high heat. If you would like to barbecue, using a sheet of aluminum foil on the grill will help decrease the food's exposure to such protein-altering high heat.

c. I put aluminum foil on the grill. (0)

Not barbecuing your food helps you avoid the carcinogens that can develop as a result of cooking meat and fish at very high heat. If you would like to barbecue, using a sheet of aluminum foil on the grill will help decrease the food's exposure to such protein-altering high heat.

d. Lightly grilled (0)

Good, you are being careful with your grilling and barbecuing by not exposing your food to extremely high temperatures. Such high temperatures can alter proteins to produce carcinogens known as heterocyclic amines. On the other hand, be careful to cook enough foods such as poultry and hamburger so that harmful bacteria such as E coli and Salmonella are killed.

e. Almost charred or charred. (−4)

The very high heat that can be present when barbecuing or grilling food can alter the protein present in meat, poultry and fish to produce carcinogens. One way to avoid this is to use aluminum foil on the grill.
* [If the person answers “a” to question 2b, they are likely to have answered “a” to question 2a. Thus if they answer yes to 2a and/or 2b, then they should only get a total of +16 points, not +32.]

3. Calcium Intake

a. 0-1 servings (−2)

Most adult men and women fall short of optimal recommended calcium intake. The bones of the human skeleton contain 99.5% of the total calcium in the body. Thus if your body needs calcium for purposes other than making bone, and there is not enough calcium in your diet, then the body will take the calcium away from your bones, thus making them weaker. Insuring that you have enough calcium and vitamin D (which facilitates the body's ability to use calcium) in your diet will help prevent this.
Adequate calcium intake in later life can slow the bone loss associated with aging. In addition to dairy products, calcium-fortified juices, breads and cereals are also excellent sources, as are calcium supplements like TUMS and over the counter calcium supplements. Vitamin D is necessary for the body to absorb the calcium we get in our diets. Milk is fortified with vitamin D and our bodies also make it when our skin is exposed to 15 to 20 minutes a day of sunlight. People who rarely go outside are prone to vitamin D deficiency. Large quantities of salty foods and meat can significantly increase the amount of calcium lost in the urine. Adequate calcium intake may not prevent the accelerated bone loss in women during and for several years after menopause, caused by estrogen deficiency. Some foods high in calcium also contain oxalic acid, which interferes with calcium absorption. Spinach is such a food.

b. 2-3 servings (+3)

Continue to do your best to insure that you are getting plenty of calcium from your diet and if needed, from a vitamin or calcium supplement. The recommended calcium intake is generally about 1,000-1,500 mg per day. In addition, be sure that you have enough vitamin D in your diet (fortified in milk, often added to calcium supplements, made by our bodies with 15-20 minutes of exposure to the sun}.

Adequate calcium intake in later life can slow the bone loss associated with aging. In addition to dairy products, calcium-fortified juices, breads and cereals are also excellent sources, as are calcium supplements like TUMS and over the counter calcium supplements. Vitamin D is necessary for the body to absorb the calcium we get in our diets. Milk is fortified with vitamin D and our bodies also make it when our skin is exposed to 15 to 20 minutes a day of sunlight. People who rarely go outside are prone to vitamin D deficiency. Large quantities of salty foods and meat can significantly increase the amount of calcium lost in the urine. Adequate calcium intake may not prevent the accelerated bone loss in women during and for several years after menopause, caused by estrogen deficiency. Some foods high in calcium also contain oxalic acid, which interferes with calcium absorption. Spinach is such a food.

c. More than 3 servings (+3)

Continue to do your best to insure that you are getting plenty of calcium from your diet and if needed, from a vitamin or calcium supplement. The recommended calcium intake is generally about 1,000-1,500 mg per day. In addition, be sure that you have enough vitamin D in your diet (fortified in milk, often added to calcium supplements, made by our bodies with 15-20 minutes of exposure to the sun}.

Adequate calcium intake in later life can slow the bone loss associated with aging. In addition to dairy products, calcium-fortified juices, breads and cereals are also excellent sources, as are calcium supplements like TUMS and over the counter calcium supplements. Vitamin D is necessary for the body to absorb the calcium we get in our diets. Milk is fortified with vitamin D and our bodies also make it when our skin is exposed to 15 to 20 minutes a day of sunlight. People who rarely go outside are prone to vitamin D deficiency. Large quantities of salty foods and meat can significantly increase the amount of calcium lost in the urine. Adequate calcium intake may not prevent the accelerated bone loss in women during and for several years after menopause, caused by estrogen deficiency. Some foods high in calcium also contain oxalic acid, which interferes with calcium absorption. Spinach is such a food.

d. I also take supplemental calcium (+3)

Excellent that you are taking supplemental calcium. The recommended calcium intake is generally 1,000-1,500 mg per day. In addition, be sure that you have enough vitamin D in your diet (fortified in milk, often added to calcium supplements).

Adequate calcium intake in later life can slow the bone loss associated with aging. In addition to dairy products, calcium-fortified juices, breads and cereals are also excellent sources, as are calcium supplements like TUMS and over the counter calcium supplements. Vitamin D is necessary for the body to absorb the calcium we get in our diets. Milk is fortified with vitamin D and our bodies also make it when our skin is exposed to 15 to 20 minutes a day of sunlight. People who rarely go outside are prone to vitamin D deficiency. Large quantities of salty foods and meat can significantly increase the amount of calcium lost in the urine. Adequate calcium intake may not prevent the accelerated bone loss in women during and for several years after menopause, caused by estrogen deficiency. Some foods high in calcium also contain oxalic acid, which interferes with calcium absorption. Spinach is such a food.

IT IS OK FOR THEM TO CHOOSE BOTH C AND D BUT WHEN IT IS SCORED, THEY SHOULD RECEIVE ONLY 3 POINTS NOT 6. If they choose d and any other answer, they should get +3.
4a. Heading for 4a and b combined: Snacking
a. Yes=0
b. No=0
4b. If you do snack between meals, generally which of the following are your snacks? Choose all that apply!
a. Fruit (+5)
b. Yogurt (+5)
c. Veggies (+5)
d. sweets (−5)
e. Bagels/chips (−5)
f. Crackers (−3)
g. Popcorn (+2)
h. “Healthy” drinks (e.g. fruit based, teas, etc.) (+3)
i. “Unhealthy” drinks (e.g. popular sodas) (−1)

They should be able to choose all that apply. Add the highest negative score to the highest positive score to come up with a net score. [After summing the above answers, the maximum negative score can be −8 (even if they score −20) and the maximum positive score can be +8 (even if they score +20)].

Resultant scores:

a. 0 to +20

Good job . . . the snacks you are eating are at least healthy. Be careful that you don't eat too much though, leading to weight gain. Be mindful of the number of calories in the foods you eat and try to not go above a healthy number of calories per day.
Nutrition experts vary on the benefits of snacking. Some say that 6 small meals a day is better than three big meals. Others indicate the opposite. You should choose a dietary habit that works best for you with the goal of not being overweight. Certainly if you do snack, be sure they are healthy snacks. Most sweets have little if any nutritional value and in the end, because they are so calorie rich from fats, they will certainly predispose you to being overweight or even heavier. Switch from those unhealthy snacks to healthy ones. Then, consider if the snacks are helping you maintain a lean body mass or not.

b. −1 to −20

Snacks are a common cause of excess calories that lead to being overweight or even obese. Add on top of that the poor nutritional value of the snacks that you are eating and you could really be doing yourself some harm. Do your best to change this habit. At least change the type of snacks you eat. Try fruit, vegetables like carrot sticks, popcorn, healthy fruit drinks etc. Even with some of these healthy foods, such as fruits and fruit drinks, you can take in significant calories so be aware of not only what you are eating but how much.
Nutrition experts vary on the benefits of snacking. Some say that 6 small meals a day is better than three big meals. Others indicate the opposite. You should choose a dietary habit that works best for you with the goal of not being overweight. Certainly if you do snack, be sure they are healthy snacks. Most sweets have little if any nutritional value and in the end, because they are so calorie rich from fats, they will certainly predispose you to being overweight or even heavier. Switch from those unhealthy snacks to healthy ones. Then, consider if the snacks are helping you maintain a lean body mass or not.
We all need to see the forest through the trees, and that it is these daily habits over time that can really impact on a person. If you can just reduce these bad habits by even one day a week, then two or three, it would make a big difference over the course of 6 months or year!
5. Red Meat Intake

a. I don't eat red meat (+8)

Good for you. A vegetarian, or at least a meatless diet is a healthy diet. Less meat in the diet is conducive to less heart disease and risk for heart attack and stroke. Furthermore, there are nutritional sources of antioxidants in foods that replace meat in a person's diet especially the polyphenols present in certain vegetables and fruits and the omega-3 fatty acids in fish that help lower LDL cholesterol and raise HDL cholesterol.
Weighing meat against vegetables and fish or skinless poultry, red meat loses out when it comes to your health. The American Heart Association recommends a diet that minimizes meat in the diet and emphasizes these alternatives. Vegetarian dishes, in addition to being an alterative to meat, also have antioxidants that protect the heart and brain. Fish contains omega-3 fatty acids, which help a person raise their good cholesterol (HDL) and lower their bad cholesterol (LDL) thus decreasing their risk for heart attack and stroke.
Interestingly, the vascular risk associated with red meat may be related to the fact that it is the major source of iron in our diet. The iron present in vegetables, like spinach, is relatively bio-unavailable. As you will read in greater detail, under the topic of iron supplements, iron plays a critical role in our cells' ability to produce harmful free radicals that likely potentiate aging and age-related illnesses.

b. I eat red meat 1-2 days per week (+4)
Good for you. A diet that minimizes meat is healthier. Less meat in the diet is conducive to less heart disease and risk for heart attack and stroke. Furthermore, there are nutritional sources of antioxidants in foods that replace meat in a person's diet especially the polyphenols present in certain vegetables and fruits and the omega-3 fatty acids in fish that help lower LDL cholesterol and raise HDL cholesterol.
Weighing meat against vegetables and fish or skinless poultry, red meat loses out when it comes to your health. The American Heart Association recommends a diet that minimizes meat in the diet and emphasizes these alternatives. Vegetarian dishes, in addition to being an alterative to meat, also have antioxidants that protect the heart and brain. Fish contains omega-3 fatty acids, which help a person raise their good cholesterol (HDL) and lower their bad cholesterol (LDL) thus decreasing their risk for heart attack and stroke.
   Interestingly, the vascular risk associated with red meat may be related to the fact that it is the major source of iron in our diet. The iron present in vegetables, like spinach, is relatively bio-unavailable. As you will read in greater detail, under the topic of iron supplements, iron plays a critical role in our cells' ability to produce harmful free radicals that likely potentiate aging and age-related illnesses.

c. I eat red meat 3-5 days per week (−12)

You are eating too much meat. Those who eat meat as often as you do are more at risk of heart disease and stroke than those who eat it less often. You are also missing out on alternatives to meat that actually can protect you from these and other diseases. For example, polyphenols present in certain vegetables and fruits, and the omega-3 fatty acids in fish that help lower LDL cholesterol and raise HDL cholesterol contribute to a more healthy cardiovascular system.
Weighing meat against vegetables and fish or skinless poultry, red meat loses out when it comes to your health. The American Heart Association recommends a diet that minimizes meat in the diet and emphasizes these alternatives. Vegetarian dishes, in addition to being an alterative to meat, also have antioxidants that protect the heart and brain. Fish contains omega-3 fatty acids, which help a person raise their good cholesterol (HDL) and lower their bad cholesterol (LDL) thus decreasing their risk for heart attack and stroke.
Interestingly, the vascular risk associated with red meat may be related to the fact that it is the major source of iron in our diet. The iron present in vegetables, like spinach, is relatively bio-unavailable. As you will read in greater detail, under the topic of iron supplements, iron plays a critical role in our cells' ability to produce harmful free radicals that likely potentiate aging and age-related illnesses.

d. I eat red meat 6-7 days per week (−24)

You are eating way too much meat. Those who eat meat as often as you do are more at risk of heart disease and stroke than those who eat it less often. You are also missing out on alternatives to meat that actually can protect you from these and other diseases. For example, polyphenols present in certain vegetables and fruits, and the omega-3 fatty acids in fish that help lower LDL cholesterol and raise HDL cholesterol contribute to a more healthy cardiovascular system.
Weighing meat against vegetables and fish or skinless poultry, red meat loses out when it comes to your health. The American Heart Association recommends a diet that minimizes meat in the diet and emphasizes these alternatives. Vegetarian dishes, in addition to being an alterative to meat, also have antioxidants that protect the heart and brain. Fish contains omega-3 fatty acids, which help a person raise their good cholesterol (HDL) and lower their bad cholesterol (LDL) thus decreasing their risk for heart attack and stroke.

6. How often to you eat sweets such as ice cream, cake/pie/pastry, or candy bars?

a. I avoid sweets (0)

Wow, such restraint! You should be proud of yourself and keep it up. Certainly, it is understandable if you have diabetes or significant heart or cerebrovascular disease (stroke and dementia) that you are staying away from sweets. By staying away from these foods that have little in the way of nutritional value and yet significantly increase propensity for obesity and therefore heart attack, stroke, cancer and diabetes, anyone will make great gains in their life expectancy and the proportion of their lives spent in good health.
Most deserts and certainly candy bars are high in saturated fats and calories. Both are terrible for you—predisposing for weight gain, heart disease, stroke and diabetes. By helping you become obese, they increase your risk of various cancers.

b. 1-2 days per week (0)

You are doing much better than the average person in demonstrating restraint from eating those deserts and candy bars. By staying away from these foods that have little in the way of nutritional value and yet significantly increase propensity for obesity and therefore heart attack, stroke, cancer and diabetes, anyone will make great gains in their life expectancy and the proportion of their lives spent in good health.
Most deserts and certainly candy bars are high in saturated fats and calories. Both are terrible for you—predisposing for weight gain, heart disease, stroke and diabetes. By helping you become obese, they increase your risk of various cancers.

c. 3-5 days per week (−6)

Avoid those checkout lines at the grocery store that sell candy bars! You are eating too many sweets and/or candy bars. By staying away from these foods that have little in the way of nutritional value and yet significantly increase propensity for obesity and therefore heart attack, stroke, cancer and diabetes, anyone will make great gains in their life expectancy and the proportion of their lives spent in good health.
Most deserts and certainly candy bars are high in saturated fats and calories. Both are terrible for you—predisposing for weight gain, heart disease, stroke and diabetes. By helping you become obese, they increase your risk of various cancers.
We all need to see the forest through the trees, and that it is these daily habits over time that can really impact on a person. If you can just reduce these bad your sweet intake by even one day a week, then two or three, it would make a big difference over the course of 6 months or year! Some people find it helpful to not eat sweets days a week and then reward themselves on two designated days, like Tuesdays and Saturdays to have a favorite dessert.

d. Once a day (−15)

It is time for you to see the forest and not the trees! You are eating too many sweets and/or candy bars. All those sweets or just one candy bar a day means 30 or more candy bars a month. Or what about 365 a year? Such a habit is very unhealthy and will shorten your life. Cutting down on sweets and doing your best to avoid them altogether will make a big difference in the health of your heart and brain. If you are overweight or obese, cutting these high cholesterol, high fat foods out of your diet will be a big step in your attempts to lose weight. By staying away from these foods that have little in the way of nutritional value and yet significantly increase propensity for obesity and therefore heart attack, stroke, cancer and diabetes, anyone will make great gains in their life expectancy and the proportion of their lives spent in good health.
Most deserts and certainly candy bars are high in saturated fats and calories. Both are terrible for you—predisposing for weight gain, heart disease, stroke and diabetes. By helping you become obese, they increase your risk of various cancers.
We all need to see the forest through the trees, and that it is these daily habits over time that can really impact on a person. If you can just reduce these bad your sweet intake by even one day a week, then two or three, it would make a big difference over the course of 6 months or year! Some people find it helpful to not eat sweets days a week and then reward themselves on two designated days, like Tuesdays and Saturdays to have a favorite dessert.
e. More than once per day (−20)

It is time for you to see the forest and not the trees! You are eating too many sweets and/or candy bars. All those sweets or just one candy bar a day means 30 or more candy bars a month. Or what about 365 a year? Such a habit is very unhealthy and will shorten your life. Cutting down on sweets and doing your best to avoid them altogether will make a big difference in the health of your heart and brain. If you are overweight or obese, cutting these high cholesterol, high fat foods out of your diet will be a big step in your attempts to lose weight. By staying away from these foods that have little in the way of nutritional value and yet significantly increase propensity for obesity and therefore heart attack, stroke, cancer and diabetes, anyone will make great gains in their life expectancy and the proportion of their lives spent in good health.
Most deserts and certainly candy bars are high in saturated fats and calories. Both are terrible for you—predisposing for weight gain, heart disease, stroke and diabetes. By helping you become obese, they increase your risk of various cancers.
We all need to see the forest through the trees, and that it is these daily habits over time that can really impact on a person. If you can just reduce these bad your sweet intake by even one day a week, then two or three, it would make a big difference over the course of 6 months or year! Some people find it helpful to not eat sweets days a week and then reward themselves on two designated days, like Tuesdays and Saturdays to have a favorite dessert.

7. Carbs

a. 3 or more servings a day (−6)

Not only are the number of calories you eat important, but the type of calories are important as well. Grains, pastas, fruits, and starchy vegetables like potatoes are the most common carbohydrate foods. Simple carbohydrates like white bread, potatoes (especially French fries), pasta, white rice and sugar as well, cause the body to produce insulin in response to elevated levels of glucose in the blood. The insulin in turn induces the storage, instead of burning, of fat. Other foods like fats, protein, and more complex carbohydrates like whole grain foods and fiber are less prone to turn on the production of insulin.

The glycemic index of food is a ranking of foods based on their immediate effect on blood glucose (blood sugar) levels and thus the production of insulin. Carbohydrate foods that breakdown quickly during digestion have the highest glycemic indexes causing blood sugar and insulin to rise fast and high. Carbohydrates that breakdown slowly, releasing glucose gradually into the blood stream, have low glycemic indexes. The lower the glycemic index, the less likely that food is going to contribute to the production of fat. There are numerous books and websites that provide the glycemic indices for foods and drinks. However the general food groups noted above are a good start in your education.

b. 1-2 servings a day (−3)

Not only are the number of calories you eat important, but the type of calories are important as well. Grains, pastas, fruits, and starchy vegetables like potatoes are the most common carbohydrate foods. Simple carbohydrates like white bread, potatoes (especially French fries), pasta, white rice and sugar as well, cause the body to produce insulin in response to elevated levels of glucose in the blood. The insulin in turn induces the storage, instead of burning, of fat. Other foods like fats, protein, and more complex carbohydrates like whole grain foods and fiber are less prone to turn on the production of insulin.

The glycemic index of food is a ranking of foods based on their immediate effect on blood glucose (blood sugar) levels and thus the production of insulin. Carbohydrate foods that breakdown quickly during digestion have the highest glycemic indexes causing blood sugar and insulin to rise fast and high. Carbohydrates that breakdown slowly, releasing glucose gradually into the blood stream, have low glycemic indexes. The lower the glycemic index, the less likely that food is going to contribute to the production of fat. There are numerous books and websites that provide the glycemic indices for foods and drinks. However the general food groups noted above are a good start in your education.

c. 1 serving every other day (0)

Not only are the number of calories you eat important, but the type of calories are important as well. Grains, pastas, fruits, and starchy vegetables like potatoes are the most common carbohydrate foods. Simple carbohydrates like white bread, potatoes (especially French fries), pasta, white rice and sugar as well, cause the body to produce insulin in response to elevated levels of glucose in the blood. The insulin in turn induces the storage, instead of burning, of fat. Other foods like fats, protein, and more complex carbohydrates like whole grain foods and fiber are less prone to turn on the production of insulin.

The glycemic index of food is a ranking of foods based on their immediate effect on blood glucose (blood sugar) levels and thus the production of insulin. Carbohydrate foods that breakdown quickly during digestion have the highest glycemic indexes causing blood sugar and insulin to rise fast and high. Carbohydrates that breakdown slowly, releasing glucose gradually into the blood stream, have low glycemic indexes. The lower the glycemic index, the less likely that food is going to contribute to the production of fat. There are numerous books and websites that provide the glycemic indices for foods and drinks. However the general food groups noted above are a good start in your education.

d. One serving twice a week (+2)

Not only are the number of calories you eat important, but the type of calories are important as well. Grains, pastas, fruits, and starchy vegetables like potatoes are the most common carbohydrate foods. Simple carbohydrates like white bread, potatoes (especially French fries), pasta, white rice and sugar as well, cause the body to produce insulin in response to elevated levels of glucose in the blood. The insulin in turn induces the storage, instead of burning, of fat. Other foods like fats, protein, and more complex carbohydrates like whole grain foods and fiber are less prone to turn on the production of insulin.

The glycemic index of food is a ranking of foods based on their immediate effect on blood glucose (blood sugar) levels and thus the production of insulin. Carbohydrate foods that breakdown quickly during digestion have the highest glycemic indexes causing blood sugar and insulin to rise fast and high. Carbohydrates that breakdown slowly, releasing glucose gradually into the blood stream, have low glycemic indexes. The lower the glycemic index, the less likely that food is going to contribute to the production of fat. There are numerous books and websites that provide the glycemic indices for foods and drinks. However the general food groups noted above are a good start in your education.

e. 1or fewer servings a week (+5)

Not only are the number of calories you eat important, but the type of calories are important as well. Grains, pastas, fruits, and starchy vegetables like potatoes are the most common carbohydrate foods. Simple carbohydrates like white bread, potatoes (especially French fries), pasta, white rice and sugar as well, cause the body to produce insulin in response to elevated levels of glucose in the blood. The insulin in turn induces the storage, instead of burning, of fat. Other foods like fats, protein, and more complex carbohydrates like whole grain foods and fiber are less prone to turn on the production of insulin.

The glycemic index of food is a ranking of foods based on their immediate effect on blood glucose (blood sugar) levels and thus the production of insulin. Carbohydrate foods that breakdown quickly during digestion have the highest glycemic indexes causing blood sugar and insulin to rise fast and high. Carbohydrates that breakdown slowly, releasing glucose gradually into the blood stream, have low glycemic indexes. The lower the glycemic index, the less likely that food is going to contribute to the production of fat. There are numerous books and websites that provide the glycemic indices for foods and drinks. However the general food groups noted above are a good start in your education.
8. Diet and Your Weight

a. I eat too much everyday, making it easy for me to stay overweight or to gain even more weight. (−15)

Cutting down on the amount you eat is one of the most important interventions you can do have a dramatic impact on your health. For many people, eating too much is the main reason they are overweight.

For other's, it is more difficult than just not eating so much and a consultation with a diet expert may be warranted. Be conscientious about how much you eat. Become a calorie counter. Don't eat till you are full. Stop before you get there. There are also several helpful websites on the internet that are dedicated to helping people lose weight and then maintain a healthy weight. See: ShapeUp America! http://www.shapeup.org/
Obesity is associated with inefficient energy production and an increased production of oxygen radicals within cells, therefore leading to increased risk of various cancers, heart disease and accelerated aging. It may also lead to diabetes.

b. I have a diet in which I am loosing weight with a target of a healthy weight. (+10)

Good for you! You are doing better or at least aiming for a goal better than more than 60% of the country who are at least overweight. Being overweight is a significant risk factor for many age-related diseases as well as various cancers. Keep up the good work. Stay lean!
Obesity is associated with inefficient energy production and an increased production of oxygen radicals within cells, therefore leading to increased risk of various cancers, heart disease and accelerated aging. It may also lead to diabetes.

c. I am maintaining a healthy weight with the diet I currently have. (+10)

Good for you! You are doing better or at least aiming for a goal better than more than 60% of the country who are at least overweight. Being overweight is a significant risk factor for many age-related diseases as well as various cancers. Keep up the good work. Stay lean!
Obesity is associated with inefficient energy production and an increased production of oxygen radicals within cells, therefore leading to increased risk of various cancers, heart disease and accelerated aging. It may also lead to diabetes.

9. Iron Intake

a. Yes (−8)

Taking an iron supplement might potentiate your aging and risk for age-related diseases. There is growing evidence from animal and human studies that iron levels are related to aging and age-associated diseases. As a critical component of mitochondrial free radical generation, iron has been proposed by some to be a key modulator of rate of aging and susceptibility to age-related diseases. Some data support the potentiating role of iron in lipid peroxidation, the first step in the formation of atherosclerotic (arterial plaque) lesions. The available epidemiological evidence suggests that elevated iron levels are involved in the pathogenesis of atherosclerosis.
Menstrual blood loss and resultant iron deficiency might be protective against vascular disease and even contribute to the premenopausal survival advantage of women over men. Further studies are needed to determine whether there are cardiovascular benefits or risks associated with blood donation.
Men may have the opportunity to be more female-like in their risk for vascular diseases by regularly donating blood, which could induce an iron deficiency. Blood donation has actually been associated with a decreased risk of atherosclerosis.

b. No (0)

It is probably good to continue to stay away from iron supplements and foods that are high in iron content, specifically red meat, if you can. Taking an iron supplement might potentiate your aging and risk for age-related diseases. There is growing evidence from animal and human studies that iron levels are related to aging and age-associated diseases. As a critical component of mitochondrial free radical generation, iron has been proposed by some to be a key modulator of rate of aging and susceptibility to age-related diseases. Some data support the potentiating role of iron in lipid peroxidation, the first step in the formation of atherosclerotic (arterial plaque) lesions. The available epidemiological evidence suggests that elevated iron levels are involved in the pathogenesis of atherosclerosis.
Menstrual blood loss and resultant iron deficiency might be protective against vascular disease and even contribute to the premenopausal survival advantage of women over men. Further studies are needed to determine whether there are cardiovascular benefits or risks associated with blood donation. Men may have the opportunity to be more female-like in their risk for vascular diseases by regularly donating blood, which could induce an iron deficiency. Blood donation has actually been associated with a decreased risk of atherosclerosis.

c. Yes, but it relieves symptoms related to my anemia (low blood count) or I am taking it temporarily after surgery (0)

If taking supplemental iron is making you feel better, than you probably should continue it. If you have symptomatic iron deficiency anemia, be sure that your physician has told you what the cause is. Unexplained iron deficiency anemia requires investigation to determine the cause. Treating the anemia with iron is not enough. Some causes of iron deficiency anemia can be very serious including cancer somewhere in the gastrointestinal tract or a stomach ulcer. The finding of iron deficiency anemia can be a very important clue to detecting and treating these and other problems effectively.
Sometimes, people have surgery in which there has been blood loss and they are told to take iron to help get their blood count back up post-surgery. Sometimes though, people are under the impression that they need to be on iron forever-after, and this is not the case. If you fit into this group, be sure to address this issue with your doctor.
Do not be on an iron supplement unnecessarily. There is growing evidence from animal and human studies that iron levels are related to aging and age-associated diseases. As a critical component of mitochondrial free radical generation, iron has been proposed by some to be a key modulator of rate of aging and susceptibility to age-related diseases. Some data support the potentiating role of iron in lipid peroxidation, the first step in the formation of atherosclerotic (arterial plaque) lesions. The available epidemiological evidence suggests that elevated iron levels are involved in the pathogenesis of atherosclerosis.

10. Exercise
a. 7 days per week=(+25)

HOORAY! You are being fantastic about your exercise. Don't forget to be balanced in how much aerobic exercise you do versus strength training. Both are very important. Also, if you are doing a lot of high impact workouts, gauge how much wear and tear you are putting on your weight-bearing joints so that you don't set yourself up for premature osteoarthritis.
Exercise leads to more efficient energy production by your cells and less oxygen radical formation (which speeds up aging and increases your risk for heart disease, stroke and cancer). Muscle is a tremendously efficient burner of fat and maintaining muscle mass has many benefits. Therefore, strength training is important. Depending upon how much one weighs, we generally loose a third of a pound of muscle every year after age 30, which is replaced by fat. Muscle loss can be completely reversed no matter what your age, by regular strengthening
b. 6 days per week=(+18)

Exercise leads to more efficient energy production by your cells and less oxygen radical formation (which speeds up aging and increases your risk for heart disease, stroke and cancer). Muscle is a tremendously efficient burner of fat and maintaining muscle mass has many benefits. Therefore, strength training is important. Depending upon how much one weighs, we generally loose a third of a pound of muscle every year after age 30, which is replaced by fat. Muscle loss can be completely reversed no matter what your age, by regular strengthening
c. 5 days per week=(+15)

Wow, you are being terrific about exercising. Don't forget to be balanced in how much aerobic exercise you do versus strength training. Both are very important. Also, if you are doing a lot of high impact workouts, gauge how much wear and tear you are putting on your weight-bearing joints so that you don't set yourself up for premature osteoarthritis.
Exercise leads to more efficient energy production by your cells and less oxygen radical formation (which speeds up aging and increases your risk for heart disease, stroke and cancer). Muscle is a tremendously efficient burner of fat and maintaining muscle mass has many benefits. Therefore, strength training is important. Depending upon how much one weighs, we generally loose a third of a pound of muscle every year after age 30, which is replaced by fat. Muscle loss can be completely reversed no matter what your age, by regular strengthening

d. 3-4 days per week=(+10)

Good job! It is great you are making the time to get that exercise in at least three times a week. Don't forget to be balanced in how much aerobic exercise you do versus strength training. Both are very important. Also, if you are doing a lot of high impact workouts, gauge how much wear and tear you are putting on your weight-bearing joints so that you don't set yourself up for premature osteoarthritis.
Exercise leads to more efficient energy production by your cells and less oxygen radical formation (which speeds up aging and increases your risk for heart disease, stroke and cancer). Muscle is a tremendously efficient burner of fat and maintaining muscle mass has many benefits. Therefore, strength training is important. Depending upon how much one weighs, we generally loose a third of a pound of muscle every year after age 30, which is replaced by fat. Muscle loss can be completely reversed no matter what your age, by regular strengthening
e. 2 days per week=(+4)

Good Job. You are already making a great effort at getting that exercise into your daily routine. If you can increase the frequency even one day to three times a week, the added benefits to your physical and mental health could be substantial. Don't forget to be balanced in how much aerobic exercise you do versus strength training. Both are very important. Also, if you are doing a lot of high impact workouts, gauge how much wear and tear you are putting on your weight-bearing joints so that you don't set yourself up for premature osteoarthritis.
Exercise leads to more efficient energy production by your cells and less oxygen radical formation (which speeds up aging and increases your risk for heart disease, stroke and cancer). Muscle is a tremendously efficient burner of fat and maintaining muscle mass has many benefits. Therefore, strength training is important. Depending upon how much one weighs, we generally loose a third of a pound of muscle every year after age 30, which is replaced by fat. Muscle loss can be completely reversed no matter what your age, by regular strengthening
f. 1 day per week=(+1)

Though exercise once a week is laudable, it is not enough. Do what you can to crank up the frequency of sustained exercise to at least three times a week. The added benefits to your physical and mental health could be substantial. Don't forget to be balanced in how much aerobic exercise you do versus strength training. Both are very important. Also, if you are doing a lot of high impact workouts, gauge how much wear and tear you are putting on your weight-bearing joints so that you don't set yourself up for premature osteoarthritis.
Exercise leads to more efficient energy production by your cells and less oxygen radical formation (which speeds up aging and increases your risk for heart disease, stroke and cancer). Muscle is a tremendously efficient burner of fat and maintaining muscle mass has many benefits. Therefore, strength training is important. Depending upon how much one weighs, we generally loose a third of a pound of muscle every year after age 30, which is replaced by fat. Muscle loss can be completely reversed no matter what your age, by regular strengthening
g. I don't exercise=(−12)

No regular exercise at all? You may have a good reason, but is it better than the reasons for why exercise would benefit you? Beginning an exercise routine and sticking to it can be as hard as changing your diet, stopping smoking etc. etc. But once you start the habit (or as in the case of smoking, stopping it), it becomes easier and easier.
Exercise leads to more efficient energy production by your cells and less oxygen radical formation (which speeds up aging and increases your risk for heart disease, stroke and cancer). Muscle is a tremendously efficient burner of fat and maintaining muscle mass has many benefits. Therefore, strength training is important. Depending upon how much one weighs, we generally loose a third of a pound of muscle every year after age 30, which is replaced by fat. Muscle loss can be completely reversed no matter what your age, by regular strengthening

D. Medical Check-up

1. Bowel Movements

a. Yes (0)

Having a bowel movement at least once every two days may be at least associated with decreased risk of colon cancer.
Keeping gut transit time under 20 hours seems to decrease the incidence of colon cancer, probably by decreasing the contact time between the gut lining and cancer-potentiating substances in the diet. These substances influence DNA damage and repair and therefore probably also influence the rate of aging as well. Epidemiological studies in humans and animal studies suggest that increasing dietary fiber will reduce the risk of certain cancers perhaps by increasing the frequency of bowel movements. On the other hand, recent reports indicate that the association may not be as clear as once believed. In addition to increased transit time and therefore less contact between carcinogens and the bowel wall, perhaps other factors that increase transit time such as regular exercise might be the real reason for decreased cancer risk.

b. No, I have a bowel movement less frequently then every two days (−4)

Having a bowel movement at least once every two days may be at least associated with decreased risk of colon cancer. Conversely, it is not clear if a decreased rate means an increased risk. Recent research has shown that determining the risk is quite complicated. None-the-less, theoretically it makes sense that an increased risk might be associated with the slower rate or something else that makes the rate slower, such as dehydration or lack of exercise.
Keeping gut transit time under 20 hours seems to decrease the incidence of colon cancer, probably by decreasing the contact time between the gut lining and cancer-potentiating substances in the diet. These substances influence DNA damage and repair and therefore probably also influence the rate of aging as well. Epidemiological studies in humans and animal studies suggest that increasing dietary fiber will reduce the risk of certain cancers perhaps by increasing the frequency of bowel movements. On the other hand, recent reports indicate that the association may not be as clear as once believed. In addition to increased transit time and therefore less contact between carcinogens and the bowel wall, perhaps other factors that increase transit time such as regular exercise might be the real reason for decreased cancer risk.
2. Female: Self-Examination for Cancer

a. Yes (+8)

Excellent. Early detection of breast cancer is so critical and monthly self-examination is an important step in catching the disease early enough.

Please refer to the American Cancer Society's website on breast cancer which discusses many aspects of the disease including how to detect it early with monthly self examination (they have a very clear set of instructions on how to do the exam) and other steps women need to take including an annual mammogram from age 40 on (or earlier, if you are at increased risk). Go to The American Cancer Society at: http://www3.cancer.org/cancerinfo/load cont.asp?ct=5&doc=15&Language=English
b. No (−8)
   A very disturbing one out of nine women get breast cancer. Don't tempt fate by ignoring this statistic. Early detection of breast cancer is so critical and monthly self-examination is an important step in catching the disease early enough. Go to the American Cancer Society's informative page on breast cancer to be sure you are examining yourself properly and that you are taking the other necessary steps to catch the disease before it catches you. The website discusses many aspects of breast cancer including how to detect it early with monthly self examination (they have a very clear set of instructions on how to do the exam) and other steps women need to take including annual mammograms. Go to The American Cancer Society at: http://www3.cancer.org/cancerinfo/load cont.asp?ct=5&doc=15&Language=English
c. I am 20 years old or younger (0)
   If your mother, aunts, or sisters have had breast cancer, even if you thought you were too young to begin self-exams and other ways of detecting breast cancer early, you should discuss this with a breast cancer specialist. Certain forms of breast cancer clearly run in families and thus starting screening at a younger age is important. Otherwise, if breast cancer doesn't run in your family, still begin monthly self-exams by age 20 and undergo an examination by a health professional at age 20 and then every three years thereafter till age 40. From age 40 on, such an exam should be performed every year.

Breast cancer is a commonly diagnosed cancer in women. A shocking one out of nine women will be diagnosed with this potentially lethal disease that when detected early can be cured. Please refer to the American Cancer Society's website on breast cancer which discusses many aspects of the disease including how to detect it early with monthly self examination (they have a very clear set of instructions on how to do the exam) and other steps women need to take including mammogram. Go to The American Cancer Society at: http://www3.cancer.org/cancerinfo/load cont.asp?ct=5&doc=15&Language=English
2. Male:
a. Yes (+8)

Good for you. Self-examination is critical in detecting this cancer before it is too late. Most testicular cancers occur between the ages of 15 and 40. But, this cancer can affect males of any age, including infants and elderly men. In about 90% of cases, men have a painless or an uncomfortable lump on a testicle, or they may notice testicular enlargement or swelling. Men with testicular cancer often report a sensation of heaviness or aching in the lower abdomen or scrotum. Make sure you are performing the exam correctly by visiting the American Cancer Society's Testicular Cancer resource Center.

http://www3.cancer.org/cancerinfo/load cont.asp?st=ds&ct=41&language=english
b. No (−8)

Self-examination is critical in detecting this cancer before it is too late. Most testicular cancers occur between the ages of 15 and 40. But, this cancer can affect males of any age, including infants and elderly men. In about 90% of cases, men have a painless or an uncomfortable lump on a testicle, or they may notice testicular enlargement or swelling. Men with testicular cancer often report a sensation of heaviness or aching in the lower abdomen or scrotum. Make sure you are performing the exam correctly by visiting the American Cancer Society's Testicular Cancer resource Center.

http://www3.cancer.org/cancerinfo/load cont.asp?st=ds&ct=41&language=english
c. I am 20 years old or younger (0)

Testicular cancer actually hits younger men most often in the range of age 15 to 40. Therefore, teenagers should be performing a testicular self-examination. Self-examination is critical in detecting this cancer before it is too late.
Most testicular cancers occur between the ages of 15 and 40. But, this cancer can affect males of any age, including infants and elderly men. In about 90% of cases, men have a painless or an uncomfortable lump on a testicle, or they may notice testicular enlargement or swelling. Men with testicular cancer often report a sensation of heaviness or aching in the lower abdomen or scrotum. Make sure you are performing the exam correctly by visiting the American,Cancer Society's Testicular Cancer resource Center.

http://www3.cancer.org/cancerinfo/load cont.asp?st=ds&ct=41&language=english
3. Cholesterol Tests
a. HDL cholesterol (good cholesterol):

I. Lower than 40 mg/dl (−10)

The first thing to do is congratulate you on knowing what your HDL level is. And second, it is good to know now, rather than later that your HDL level is too low. The sooner you can correct the situation the better. There are a number of steps that you can take to lower your LDL level, including diet, exercise, not smoking, weight loss and certain medications.
HDL cholesterol is known as the “good” cholesterol because a high level of HDL cholesterol appears to protect against heart attack. Medical experts think that HDL tends to carry cholesterol away from the arteries and back to the liver, where it is processed, dumped in the intestine and then passed from the body. Some experts believe that excess cholesterol is removed from atherosclerotic plaque by HDL, thus slowing the build-up. However, low HDL cholesterol levels (lower than 35 mg/dL) may result in a greater risk for heart disease and stroke. For more information about cholesterol, other risk factors and treatment, go to the American Heart Association's website at: http://www.americanheart.org/

II. Higher than 40 mg/dl (+10)

Good job on knowing what your level is! And, congratulations on having a level that places you at lower risk for developing atherosclerosis or plaque in the arteries. However, there are other risk factors as well.
HDL cholesterol is known as the “good” cholesterol because a high level of HDL cholesterol appears to protect against heart attack. Medical experts think that HDL tends to carry cholesterol away from the arteries and back to the liver, where it is processed, dumped in the intestine and then passed from the body. Some experts believe that excess cholesterol is removed from atherosclerotic plaque by HDL, thus slowing the build-up. However, low HDL cholesterol levels (lower than 35 mg/dL) may result in a greater risk for heart disease and stroke. For more information about cholesterol, other risk factors and treatment, go to the American Heart Association's website at: http://www.americanheart.org/

III. I haven't checked it in the last 3 years (−2)

Not knowing what your HDL level should be considered a risk factor for heart disease and stroke. It means you are not availing yourself of some key information to decrease your risk for these diseases.
HDL cholesterol is known as the “good” cholesterol because a high level of HDL cholesterol appears to protect against heart attack. Medical experts think that HDL tends to carry cholesterol away from the arteries and back to the liver, where it is processed, dumped in the intestine and then passed from the body. Some experts believe that excess cholesterol is removed from atherosclerotic plaque by HDL, thus slowing the build-up. However, low HDL cholesterol levels (lower than 35 mg/dL) may result in a greater risk for heart disease and stroke. For more information about cholesterol, other risk factors and treatment, go to the American Heart Association's website at: http://www.americanheart.org/

IV. I have had the test done within the past 3 years but don't remember the results (−1)

Not knowing what your HDL level should be considered a risk factor for heart disease and stroke. It means you are not availing yourself of some key information to decrease your risk for these diseases.
HDL cholesterol is known as the “good” cholesterol because a high level of HDL cholesterol appears to protect against heart attack. Medical experts think that HDL tends to carry cholesterol away from the arteries and back to the liver, where it is processed, dumped in the intestine and then passed from the body. Some experts believe that excess cholesterol is removed from atherosclerotic plaque by HDL, thus slowing the build-up. However, low HDL cholesterol levels (lower than 35 mg/dL) may result in a greater risk for heart disease and stroke. For more information about cholesterol, other risk factors and treatment, go to the American Heart Association's website at: http://www.americanheart.org/

b. LDL cholesterol (bad cholesterol):

I. Lower than 130 mg/dl (+7)

Good job on knowing what your level is! And, congratulations on having a level that places you at lower risk for developing atherosclerosis or plaque in the arteries. However, there are other risk factors as well.
A high level (higher than 130 mg/dL) of low-density lipoprotein, or LDL cholesterol, reflects an increased risk of heart attack and stroke. That's why LDL cholesterol is often called “bad” cholesterol. When too much LDL cholesterol circulates in the blood, it can lead to the formation of plaque in the blood vessels that feed the heart and the brain. When enough of this blockage occurs, a heart attack or stroke can occur. To learn more about risk factors you can modify, go to the American Heart Association's website at: http://www.americanheart.org/catalog/Health catpage12.html

II. Higher than 130 mg/dl (−7)

Good job on knowing what your level is! That is a first important step. But then you might also know that people with a LDL level 135 or higher are at increased risk of developing plaque in the blood vessels that lead to the heart and brain, thus making them at higher risk of developing heart attack or stroke. There are a number of steps that you can take to lower your LDL level, including diet, exercise, not smoking, weight loss and certain medications.
A high level (higher than 130 mg/dL) of low-density lipoprotein, or LDL cholesterol, reflects an increased risk of heart attack and stroke. That's why LDL cholesterol is often called “bad” cholesterol. When too much LDL cholesterol circulates in the blood, it can lead to the formation of plaque in the blood vessels that feed the heart and the brain. When enough of this blockage occurs, a heart attack or stroke can occur. To learn more about risk factors you can modify, go to the American Heart Association's website at: http://www.americanheart.org/catalog/Health catpage12.html

III. I haven't checked it in the last 3 years (−2)

It is apparent you are interested in your health and future well-being because you are taking this questionnaire. Now you need to take the next steps that include diligent screening. Get your cardiovascular disease risk factors, including your LDL cholesterol level, checked.
A high level (higher than 130 mg/dL) of low-density lipoprotein, or LDL cholesterol, reflects an increased risk of heart attack and stroke. That's why LDL cholesterol is often called “bad” cholesterol. When too much LDL cholesterol circulates in the blood, it can lead to the formation of plaque in the blood vessels that feed the heart and the brain. When enough of this blockage occurs, a heart attack or stroke can occur. To learn more about risk factors you can modify, go to the American Heart Association's website at: http://www.americanheart.org/catalog/Health catpage12.html

IV. I have had the test done within the past 3 years but don't remember the results (−1)

Well at least you had your level checked—that's good. And maybe you don't remember the level because all you do recall is that the level was “normal”. That's good news! But if you don't remember at all, give your health professional a call and find out what the level was and when you are due for another assessment.
A high level (higher than 130 mg/dL) of low-density lipoprotein, or LDL cholesterol, reflects an increased risk of heart attack and stroke. That's why LDL cholesterol is often called “bad” cholesterol. When too much LDL cholesterol circulates in the blood, it can lead to the formation of plaque in the blood vessels that feed the heart and the brain. When enough of this blockage occurs, a heart attack or stroke can occur. To learn more about risk factors you can modify, go to the American Heart Association's website at: http://www.americanheart.org/catalog/Health catpage12.html

c. Cholesterol level:

I. Lower than 180 mg/dl (+3)

Good job on knowing what your level is! And, congratulations on having a level that places you at lower risk for developing atherosclerosis or plaque in the arteries.

II. Higher than 180 mg/dl (−8)

Well at least you know your level. That is definitely a step in the right direction. Now you need to take steps to lower your triglyceride level. Many can do this with diet, exercise, and medications. Some people are born with an inheritable condition that causes very high and dangerous levels.
For more information about cholesterol go to the American Heart Association's Website at: http://www.americanheart.org/presenter.jhtml?identifier=1516

III. I haven't checked it in the last 3 years (−2)

Knowing your risk for cardiovascular disease and stroke is important if you are going to positively impact your health span. Some of these factors that increase your risk could be high even if you think you have a healthy diet and you exercise regularly. Thus, you need to see your health professional and get your blood tested. For more information about cholesterol go to the American Heart Association's Website at: http://www.americanheart.org/presenter.jhtml?identifier=1516

IV. I have had the test done within the past 3 years but don't remember the results (−1)

Maybe all you remember is that your doctor said your level was normal. That would be great except that “normal” varies according to whom you listen to. “Normal” for the American diet might be 200 mg/dl. However, as we all know, the normal American diet leads to heart disease at near epidemic rates. Find out what your level was and find out when you need to have it checked again. Then come back and retake this quiz. For more information about cholesterol go to the American Heart Association's Website at: http://www.americanheart.org/presenter.jhtml?identifier=1516

For more information about cholesterol go to the American Heart Association's Website at: http://www.americanheart.org/presenter.jhtml?identifier=1516
4. Blood Pressure
a. Systolic blood pressure (higher value):

I. Lower than 85 (−5) A systolic pressure less than 85 is likely to be a mistake or an isolated reading when you were not feeling well. Usually, a sustained reading that low would indicate that a person is in shock and very sick. If you are on blood pressure medications and you truly have a systolic this low, then you need urgently adjustments in your medications.
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

II. 86-100 (+6) You have what many would call a low blood pressure. Usually it gets too low if you are on blood pressure medication (you may be getting over-medicated). On the other hand, if you are not on blood pressure medications, this level is probably conducive to longevity. Centenarians generally have a history of a low blood pressure for most of their lives.
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

III. 101-129 (0) You have a normal systolic blood pressure. Exercise, stress management and, if you are overweight, getting your weight down will probably lower it . . . the closer to 100-110 the better.
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

IV. 130-139 (−5) A systolic of 130 to 139 is borderline elevated. You and your health professional need to keep on eye on this. Try to get your pressure down with weight loss if you are overweight, a healthy diet, stress management and exercise. If your pressure continues to increase, you may need to bring it down with medication.
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

V. 140-189 (−12)

If repeated measurements of your systolic blood pressure are this high than you need to get your blood pressure down to a healthy range, at least less than 140 and optimally in the 110 range. Discuss this with your health care professional, but certainly getting your weight down if you are overweight, exercise and stress management are important. Depending upon your success with these measures, you may need a medication.
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

VI. 190-210 (−24)

Your systolic blood pressure is too high and you need to get your blood pressure down to a healthy range, at least less than 140. You hopefully have already discussed this with your health care professional; certainly getting your weight down if you are overweight, exercise and stress management are important. Depending upon your success with these measures, you may well need a medication(s).
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

VII. 211-230 (−48)

Your systolic blood pressure is much too high and you need to get your blood pressure down to a healthy range, to begin with at least less than 190 and eventually/hopefully less than 140. You hopefully have already discussed this with your health care professional; certainly getting your weight down if you are overweight, exercise and stress management are important. Medications will probably be necessary if you are not on them already.
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

VIII. Higher than 230 (−72)

If you truly have a systolic blood pressure this high (230 or greater), stop taking this quiz and get yourself to an emergency room or be seen by your health care professional today, ASAP. No kidding . . . You more than likely need to be on blood pressure lowering medication. Currently your pressure puts you at significant and immediate risk of heart attack and stroke.
Blood pressure results from two forces. The first force is created as your heart pumps blood into the arteries and through the circulatory system and this is the systolic pressure. The second is created as the arteries resist the blood flow and this is the diastolic pressure. A systolic blood pressure greater than 140 is considered high, meaning that the person is at increased risk for heart attack and stroke. The risk climbs thereafter in proportion to the pressure.

IX. I don't know (0) [Here, they should have gone to question C, where the person says they don't know . . . they should receive the feedback noted there and not receive any feedback for question a]
b. Diastolic blood pressure (lower value):

I. Lower than 80 (0)

You have a good diastolic blood pressure. Centenarians generally have a history of a diastolic blood pressure in this range for most of their lives.

II. 80-89: (−1)

You have a normal diastolic blood pressure. Exercise, stress management and, if you are overweight, getting your weight down will probably lower it . . . the closer to the 70-80 range the better.

III. 90-105: (−6)

If repeated measurements of your diastolic blood pressure are this high than you need to do something about it. If your diastolic is above 100, many health practitioners would elect to treat with medication if other non-medication (stop smoking, lose weight if you are overweight, exercise) strategies don't work.

IV. 106-115: (−12)

Your diastolic blood pressure is too high and you need to get it down to a healthy range, at least less than 90. You hopefully have already discussed this with your health care professional; certainly getting your weight down if you are overweight, exercise and stress management are important. Depending upon your success with these measures, you may well need a medication(s).

V. Higher than 116: (−24)

If you truly have a diastolic blood pressure this high (116 or greater), stop taking this quiz and get yourself to an emergency room or be seen by your health care professional today, ASAP. No kidding . . . You more than likely need to be on blood pressure lowering medication. Currently your pressure puts you at significant and immediate risk of heart attack and stroke.

VI. I don't know (0) [Here, they should have gone to question C, where the person says they don't know . . . they should receive the feedback noted there and not receive any feedback for question b]
c. I have not had my blood pressure checked in the past year (−6)

High blood pressure, otherwise known as hypertension is a silent killer. Unless the pressure is very, very high, you may not have any symptoms. Hypertension if caught early and before it gets to high is curable in many cases through pharmacological and non-pharmacological means. Thus, it behooves you to get your blood pressure checked. Please don't put this off.
5. Diabetes

a. I have not had it checked in the past 3 years) (−2)

Like high blood pressure, a person can have diabetes for a long time and not know it, thus it is very important that children and adults are regularly screened. Get your blood sugar checked at least every three years and watch for some of the more common symptoms of diabetes including excessive and persistent thirst, voiding large amounts of urine frequently with normal amounts of fluid intake, unexplained weight loss.

b. No diabetes (<120) (0)

Of course, it is great that you don't have diabetes. Lower your risk by loosing weight if you are overweight. Get your blood sugar checked at least every three years and watch for some of the more common symptoms of diabetes including excessive and persistent thirst, voiding large amounts of urine frequently with normal amounts of fluid intake, unexplained weight loss.
Diabetes occurs because a person's body does not make enough insulin and/or because the cells and tissues in their body are relatively resistant to the insulin they produce (and so the insulin is less effective). As a result, diabetics can have large amounts of glucose in their circulation. By far and away, the more common form of diabetes is Adult Onset Diabetes and this is often associated with obesity. This form is often well controlled (meaning the blood glucose level is kept normal) with weight loss, diet and/or oral medication. Sometimes insulin injections are necessary. The other form of diabetes is called juvenile diabetes, which more often occurs in children and requires insulin injections.
Good control has been shown to decrease risk of heart and kidney disease and diabetic eye problems, all unfortunate problems associated with diabetes. Like high blood pressure, a person can have diabetes for a long time and not know it, thus it is very important that children and adults are regularly screened for it. The good news is that people are leaving long, productive and basically otherwise normal lives given the excellent treatments available. Watch for some of the more common symptoms of diabetes including excessive and persistent thirst, voiding large amounts of urine frequently with normal amounts of fluid intake, unexplained weight loss. Take a diabetes risk test: American Diabetes Association: http://www.diabetes.org/ada/risktest.asp

c. 120-200 (−6)

It is important that you are controlling your blood sugars. Of course sugars in the 120-150 range are preferable to greater than 150. Studies have shown that tighter control of glucose levels approaching normal levels lowers risk of complications due to diabetes such as heart attack and kidney problems.
Diabetes occurs because a person's body does not make enough insulin and/or because the cells and tissues in their body are relatively resistant to the insulin they produce (and so the insulin is less effective). As a result, diabetics can have large amounts of glucose in their circulation. By far and away, the more common form of diabetes is Adult Onset Diabetes and this is often associated with obesity. This form is often well controlled (meaning the blood glucose level is kept normal) with weight loss, diet and/or oral medication. Sometimes insulin injections are necessary. The other form of diabetes is called juvenile diabetes, which more often occurs in children and requires insulin injections.
Good control has been shown to decrease risk of heart and kidney disease and diabetic eye problems, all unfortunate problems associated with diabetes. Like high blood pressure, a person can have diabetes for a long time and not know it, thus it is very important that children and adults are regularly screened for it. The good news is that people are leaving long, productive and basically otherwise normal lives given the excellent treatments available. Watch for some of the more common symptoms of diabetes including excessive and persistent thirst, voiding large amounts of urine frequently with normal amounts of fluid intake, unexplained weight loss. Take a diabetes risk test: American Diabetes Association: http://www.diabetes.org/ada/risktest.asp

d. >200 (−18)

It is important for you to better control your blood sugars. Of course sugars in the 120-150 range are preferable to greater than 150. Studies have shown that tighter control of glucose levels approaching normal levels lowers risk of complications due to diabetes such as heart attack and kidney problems.
Diabetes occurs because a person's body does not make enough insulin and/or because the cells and tissues in their body are relatively resistant to the insulin they produce (and so the insulin is less effective). As a result, diabetics can have large amounts of glucose in their circulation. By far and away, the more common form of diabetes is Adult Onset Diabetes and this is often associated with obesity. This form is often well controlled (meaning the blood glucose level is kept normal) with weight loss, diet and/or oral medication. Sometimes insulin injections are necessary. The other form of diabetes is called juvenile diabetes, which more often occurs in children and requires insulin injections.
Good control has been shown to decrease risk of heart and kidney disease and diabetic eye problems, all unfortunate problems associated with diabetes. Like high blood pressure, a person can have diabetes for a long time and not know it, thus it is very important that children and adults are regularly screened for it. The good news is that people are leaving long, productive and basically otherwise normal lives given the excellent treatments available. Watch for some of the more common symptoms of diabetes including excessive and persistent thirst, voiding large amounts of urine frequently with normal amounts of fluid intake, unexplained weight loss. Take a diabetes risk test: American Diabetes Association: http://www.diabetes.org/ada/risktest.asp

6. Heart Attack

a. No heart attack(O)

Great news that you have not had a heart attack. If you have been avoiding or minimizing the risk factors for heart attack (also called heart disease or coronary artery disease). If you have some of the risk factors, then you have been lucky thus far that you haven't had a heart attack. If you are young, then you should know that 90% of heart attack victims have at least one of the following risk factos so you might be setting yourself up for a heart attack in the future. Here are the typical risk factors that you can modify: high cholesterol, high blood pressure, diabetes (improve sugar control), being overweight or cigarette smoking

b. Yes, I had a heart attack more than 2 years ago, and I took action to reduce my risk factors for another one (regular exercise, stop smoking, lose weight, changed diet) (−4)

Good for you that you took action to reduce your risk factors for heart disease. It is reassuring that you are two years out from your heart attack without another one. Here are the typical risk factors that you can modify: high cholesterol, high blood pressure, diabetes (improve sugar control), being overweight, or cigarette smoking.

c. Yes, I had a heart attack within the past 2 years, and I took action to reduce my risk factors for another one (regular exercise, stop smoking, lose weight, changed diet) (−7)

Good for you that you took action to reduce your risk factors for heart disease. It is reassuring that you are two years out from your heart attack without another one. Here are the typical risk factors that you can modify: high cholesterol, high blood pressure, diabetes (improve sugar control), being overweight or cigarette smoking.

d. Yes, I had a heart attack within the past 2 years, and I HAVE NOT taken action to reduce my risk factors for another one (regular exercise, stop smoking, lose weight, changed diet) (−15)

Please don't tempt fate. You have already had a heart attack and without modifying your risk factors, even if you have had bypass surgery or angioplasty, you continue to be at increased risk for another heart attack. Here are the typical risk factors that you can modify: high cholesterol, high blood pressure, diabetes (improve sugar control), being overweight or cigarette smoking.

7. Check-Ups

a. My last preventive care visit was over 3 years ago. −5

Regular checkups are so important. The frequency of checkups depends on your age and what if any medical or other clinically relevant problems you might have. Generally, anyone age 40 or older should be having annual checkups. Please set up an appointment for a checkup as soon as possible. A conscientious program of screening and prevention are key to living into old age in good health.
b. My last preventive care visit was between 1-3 years ago. −1

Regular checkups are so important. The frequency of checkups depends on your age and what if any medical or other clinically relevant problems you might have. Generally, anyone age 40 or older should be having annual checkups.
c. My last preventive care visit was between within the past year. +2

Regular checkups are so important. The frequency of checkups depends on your age and what if any medical or other clinically relevant problems you might have. Generally, anyone age 40 or older should be having annual checkups.

E. FAMILY HISTORY
1. FAMILY HISTORY OF HEART ATTACK OR DIABETES

a. None (0)

You are lucky to not have heart attack or diabetes running in your family. Now do a good job of taking advantage, of those genes and don't do things that could predispose you to the disease nonetheless, such as being obese, smoking and not exercising regularly.

b. One (−2)

Heart attack and/or diabetes appears to run in your family. You should be extra diligent about screening for and minimizing risk factors that make a heat attack or stroke more likely. Modifiable risk factors for heart attack include: high cholesterol, high blood pressure, diabetes (improve sugar control), being overweight or cigarette smoking.

### C. TWO (−4)

Heart attack and/or diabetes appears to run in your family. You should be extra diligent about screening for and minimizing risk factors that make a heat attack or stroke more likely. Modifiable risk factors for heart attack include: high cholesterol, high blood pressure, diabetes (improve sugar control), being overweight or cigarette smoking.

### D. THREE AND MORE: (−6)

Heart attack and/or diabetes appears to run strongly in your family. You should be extra diligent about screening for and minimizing risk factors that make a heat attack or stroke more likely. Modifiable risk factors for heart attack include: high cholesterol, high blood pressure, diabetes (improve sugar control), being overweight or cigarette smoking.

e. Don't know (0)

If you don't know your family's health history, you should, just in case, be extra-diligent about minimizing modifiable risk factors for heart disease and stroke. The modifiable risk factors include high cholesterol, high blood pressure, diabetes (improve sugar control), being overweight or cigarette smoking.
2. Family History of Cancer

### A. NONE (+5)

You are lucky to apparently not have cancer running in your family. Now do a good job of taking advantage of those genes and don't do things that could predispose you to cancer nonetheless, such as being obese, smoking, etc.

### B. ONE (−4)

Your family history is warning you to be extra diligent about screening for various cancers and to do what you can to prevent it. Speak with your health care provider about what you can do to screen for cancer and catch it before it catches you.

### C. TWO (−6)

Your family history is warning you to be extra diligent about screening for various cancers and to do what you can to prevent it. Speak with your health care provider about what you can do to screen for cancer and catch it before it catches you.

### D. THREE AND MORE: (−12)

Your family history is warning you to be extra diligent about screening for various cancers and to do what you can to prevent it. Speak with your health care provider about what you can do to screen for cancer and catch it before it catches you.

e. don't know (0) If you don't know your family's health history, you should, just in case, be extra-diligent about minimizing modifiable risk factors for cancer. The modifiable risk factors include being overweight or cigarette smoking. Be sure to screen for cancer in conjunction with your health care provider.
3. Family history for longevity
a) Mother:
   1. Under the age of 80/healthy (0)

It is great that your mom is healthy. Given that she is less than 80, it is still a bit hard to predict her longevity and therefore form some idea of her contribution to your longevity
2. Age 80-89 and healthy (+6)

It is great news to learn that your mom is doing well in her 80's. This could be indicative of longevity running in your family. This would be even more the case of course if she was/is healthy in her nineties or even older!
3. AGE 90-94 AND HEALTHY (+12)

It is great news to learn that your mom is doing well in her early 90's. This will likely contribute to your own longevity.
4. AGE 95-99, NO MATTER WHAT HEALTH (+24)

### IT IS GREAT NEWS TO LEARN THAT YOUR MOM IS DOING WELL IN HER LATE 90'S, YOU, AS A RESULT, GET A SUBSTANTIAL BOOST TO YOUR LONGEVITY POTENTIAL. YOU OWE YOUR MOM A BIG HUG AND KISS!

5. AGE 100+, NO MATTER WHAT HEALTH (+60)

### IT IS GREAT NEWS TO LEARN THAT YOUR MOM IS A CENTENARIAN!!! THIS IS PRACTICALLY THE BIGGEST BOOST ONE CAN GET TO INCREASE THEIR LONGEVITY, YOU OWE YOUR MOM A BIG HUG AND KISS!

6. MOTHER HAS OR DIED OF A SMOKING RELATED (CANCER, HEART ATTACK, STROKE,
   EMPHYSEMA) ILLNESS OR TRAUMA PRIOR TO AGE 80 (−1)

Unfortunately, it is difficult to gauge the longevity in your family, at least regarding your mother, because she smoked or died prematurely because of trauma. One concern might be if she did smoke, whether or not her smoking harmed you because of your exposure to second-hand smoke.
7. Under the age of 80 and dependent on others (−8)

Because your mother is not doing so well before the age of 80, there is a concern that health problems can occur prematurely in your family. This is information to take advantage of. Be extra diligent about prevention and screening.
8. Age 81-89 and dependent on others (0)

Though your mom is not doing so well after age 80, understanding what degree of longevity runs in your family depends upon how well she has taken care of herself. Diet, exercise, stress management and other factors play an important role. Nonetheless, this is not as good news (for many reasons) as if she was doing well after age 80 or even older age.
9. Age 90-94, dependent on others (+5)

I'm sorry that your mom isn't doing so well now that she is in her early 90's. Though she was probably in pretty good health up through her mid-80s. She has demonstrated some longevity that has likely been passed down to you. Take good care of yourself and you will likely get to even older age than her and in better health and with better function.
10. Died before age 80 (not from trauma or accident, see option 6) (−12)

Because your mother died before age 80, there is a concern that health problems can occur prematurely in your family. This is information to take advantage of. Be extra diligent about prevention and screening.
11. Died age 80-89 (0)

Your mother lived to the average potential life expectancy thus it is likely she took good care of herself, or she had bad habits but also had some genes or other behaviors that made up for those habits. Given her average longevity, this makes it more likely that you also can get to your mid to late eighties if you take good care of yourself.
12. Died age 90-94 (+5)

Your mother lived to a bit more than the average potential life expectancy thus it is likely she took good care of herself, or she had bad habits but also had some genes or other behaviors that made up for those habits. Given her better than average longevity, this makes it more likely that you also can get to your mid to late eighties nineties if you take good care of yourself.
13. Died age 95-99 (+24)

Wow, your mom got pretty far up there in terms of longevity. This is likely going to give you a substantial advantage in terms of your own potential of becoming a nonagenarian or even centenarian! If she got to her age despite bad health habits than this is even better news for you.
14. Died age 100 or older (+60)

Ahhhh, your mom was a centenarian! Of course you can imagine how we love to hear about centenarians around here. That is of course great for you. This is likely going to give you a substantial advantage in terms of your own potential of becoming a centenarian! If she got to her age despite bad health habits than this is even better news for you. The children of a centenarian have a 60% reduced risk, compared to others born around the same time, for heart disease, stroke, diabetes and a 20% reduced overall mortality.
15. Adopted, don't know (0)

Unfortunately, it is not possible to gauge the longevity in your family because you were adopted.
b) Father:
   4. Under the age of 80/healthy (0)

It is great that your dad is healthy. Given that he is less than 80, it is still a bit hard to predict his longevity and therefore form some idea of his contribution to your longevity
5. Age 80-89 and healthy (+6)

It is great news to learn that your dad is doing well in his 80's. This could be indicative of longevity running in your family. This would be even more the case of course if he was/is healthy in his nineties or even older!
6. AGE 90-94 AND HEALTHY (+12)

It is great news to learn that your dad is doing well in his early 90's. This will likely contribute to your own longevity.
4. AGE 95-99, NO MATTER WHAT HEALTH (+24)

### IT IS GREAT NEWS TO LEARN THAT YOUR DAD IS DOING WELL IN HIS LATE 90'S, YOU, AS A RESULT, GET A SUBSTANTIAL BOOST TO YOUR LONGEVITY POTENTIAL. YOU OWE YOUR DAD A BIG HUG AND KISS!

5. AGE 100+, NO MATTER WHAT HEALTH (+60)

### IT IS GREAT NEWS TO LEARN THAT YOUR DAD IS A CENTENARIAN!!! THIS IS PRACTICALLY THE BIGGEST BOOST ONE CAN GET TO INCREASE THEIR LONGEVITY. YOU OWE YOUR DAD A BIG HUG AND KISS!

6. FATHER HAS OR DIED OF A SMOKING RELATED (CANCER, HEART ATTACK, STROKE,
   EMPHYSEMA) ILLNESS OR TRAUMA PRIOR TO AGE 80 (−1)

Unfortunately, it is difficult to gauge the longevity in your family, at least regarding your father, because he smoked or died prematurely because of trauma. One concern might be if he did smoke, whether or not his smoking harmed you because of your exposure to second-hand smoke.
7. Under the age of 80 and dependent on others (−8)

Because your father is not doing so well before the age of 80, there is a concern that health problems can occur prematurely in your family. This is information to take advantage of. Be extra diligent about prevention and screening.
8. Age 81-89 and dependent on others (0)

Though your dad is not doing so well after age 80, understanding what degree of longevity runs in your family depends upon how well he has taken care of himself. Diet, exercise, stress management and other factors play an important role. Nonetheless, this is not as good news (for many reasons) as if he was doing well after age 80 or even older age.
9. Age 90-94, dependent on others (+5)

I'm sorry that your dad isn't doing so well now that he is in his early 90's. Though he was probably in pretty good health up through his mid-80s. He has demonstrated some longevity that has likely been passed down to you. Take good care of yourself and you will likely get to even older age than his and in better health and with better function.
10. Died before age 80 (not from trauma or accident, see option 6) (−12)

Because your father died before age 80, there is a concern that health problems can occur prematurely in your family. This is information to take advantage of. Be extra diligent about prevention and screening.
11. Died age 80-89 (0)

Your father lived to the average potential life expectancy thus it is likely he took good care of himself, or he had bad habits but also had some genes or other behaviors that made up for those habits. Given his average longevity, this makes it more likely that you also can get to your mid to late eighties if you take good care of yourself.
12. Died age 90-94 (+5)

Your father lived to a bit more than the average potential life expectancy thus it is likely he took good care of himself, or he had bad habits but also had some genes or other behaviors that made up for those habits. Given his better than average longevity, this makes it more likely that you also can get to your mid to late eighties nineties if you take good care of yourself.
13. Died age 95-99 (+24)

Wow, your dad got pretty far up there in terms of longevity. This is likely going to give you a substantial advantage in terms of your own potential of becoming a nonagenarian or even centenarian! If he got to his age despite bad health habits than this is even better news for you.
14. Died age 100 or older (+60)

Ahhhh, your dad was a centenarian! Of course you can imagine how we love to hear about centenarians around here. That is of course great for you. This is likely going to give you a substantial advantage in terms of your own potential of becoming a centenarian! If he got to his age despite bad health habits than this is even better news for you. The children of a centenarian have a 6.0% reduced risk, compared to others born around the same time, for heart disease, stroke, diabetes and a 20% reduced overall mortality.
15. Adopted, don't know (0)

Unfortunately, it is not possible to gauge the longevity in your family because you were adopted.

4. Grandparents: Did any of your grandparents or great-grandparents live to age 98 years or older?
a. Yes (+36)
Exceptional longevity runs strongly in families. Some of that may be genes and some may be health and cultural habits that family members have in common. It is not known yet how much a grandparent's longevity contributes to their grandchild's longevity, but it is very likely that the contribution is enough to improve your own chances of getting to very old age, much of it in good health.
b. No (0)
A grandparent's longevity probably is a survival advantage for their grandparents. How much of an advantage though, is unclear. Perhaps your grandparents are alive and are heading in the direction of age 98 and older . . . that is great! But you can't get credit for this till they get there . . . so cheer them on! If your grandparents are no longer alive, at least continue to take as good care of yourself as you can to enhance your chances of at least getting to your mid to late eighties in good health and with good function,

NOTE!!! THE MAXIMUM SCORE ATTAINABLE FOR THE SUM OF QUESTIONS 2 through 4 is +120 (even if they got 161!) THE MAXIMUM NEGATIVE SCORE IS −12

[Please show the following question only if the user is a woman and age 35 or older.]
5. Maternal Age
a. I have not had any children and am still having my periods (0)
We use maternal age (when you last had a child) to gauge how slowly and well your reproductive system is aging, which tells us something about how the rest of you is doing. If you don't have children in the future, then perhaps the age at which you go through menopause will be a good marker, though there have been no studies thus far linking older age at menopause with exceptional longevity.
b. How old were you when you last had a child without fertility technology assistance? ______ years

RESULTS:

If they answer age 35-39 or older, give (+5) Having a child without fertility assistance after age 35 probably means that your reproductive system and therefore the rest of you is aging relatively slowly and that you are less prone to age related diseases that could impact upon fertility (such as diabetes).
In a study of over 100 centenarians, the Harvard based New England Centenarian Study found that 20% of the women had at least one of their children after the age of 40. This was compared to about 5% of women who lived to just average life expectancy. Thus, a woman who has a child after the age of 40 (without fertility assistance) is about 5 times more likely to achieve exceptional old age. There is also an increased probability in the case of having a child between the ages of 35 and 40, though it is less. It is probably not the act of having a child in her forties that helps get a woman to 100. Rather, this is probably a sign that her reproductive system is very health and aging very slowly and therefore the rest of her is probably aging slowly as well. Furthermore, she probably has an increased resistance to diseases associated with aging that might also impact upon fertility, such as heart disease and diabetes. Thus, do not purposefully put off having a child until you are forty or older because you think it will help you live to 100. In actuality, for most women, fertility markedly falls after age 35 and the risk to both mother and child increases after that age.

If they answer age 40-45, give (+10)

Having a child without fertility assistance after age 40 probably means that your reproductive system and therefore the rest of you is aging relatively slowly and that you are significantly less prone to age related diseases that could impact upon fertility (such as diabetes). Your chances of getting to exceptional old age are actually five times greater than someone who was infertile before age 35.
In a study of over 100 centenarians, the Harvard based New England Centenarian Study found that 20% of the women had at least one of their children after the age of 40. This was compared to about 5% of women who lived to just average life expectancy. Thus, a woman who has a child after the age of 40 (without fertility assistance) is about 5 times more likely to achieve exceptional old age. There is also an increased probability in the case of having a child between the ages of 35 and 40, though it is less. It is probably not the act of having a child in her forties that helps get a woman to 100. Rather, this is probably a sign that her reproductive system is very health and aging very slowly and therefore the rest of her is probably aging slowly as well. Furthermore, she probably has an increased resistance to diseases associated with aging that might also impact upon fertility, such as heart disease and diabetes. Thus, do not purposefully put off having a child until you are forty or older because you think it will help you live to 100. In actuality, for most women, fertility markedly falls after age 35 and the risk to both mother and child increases after that age.

If they answer age >45, give (+15)

Having a child without fertility assistance after age 45 is truly remarkable and means that your reproductive system and therefore the rest of you is aging exceptionally slowly and that you are significantly less prone to age related diseases that could impact upon fertility (such as diabetes). Your chances of getting to exceptional old age are actually five times greater (at least!) than someone who was infertile before age 35.
In a study of over 100 centenarians, the Harvard based New England Centenarian Study found that 20% of the women had at least one of their children after the age of 40. This was compared to about 5% of women who lived to just average life expectancy. Thus, a woman who has a child after the age of 40 (without fertility assistance) is about 5 times more likely to achieve exceptional old age. There is also an increased probability in the case of having a child between the ages of 35 and 40, though it is less. It is probably not the act of having a child in her forties that helps get a woman to 100. Rather, this is probably a sign that her reproductive system is very health and aging very slowly and therefore the rest of her is probably aging slowly as well. Furthermore, she probably has an increased resistance to diseases associated with aging that might also impact upon fertility, such as heart disease and diabetes. Thus, do not purposefully put off having a child until you are forty or older because you think it will help you live to 100. In actuality, for most women, fertility markedly falls after age 35 and the risk to both mother and child increases after that age.

If they answer age 34 or younger, give (0)

c. I had no children, but I had my last period (or had a hysterectomy) at age ______ years

RESULTS:

If they answer age <40, give (−5)

You stopped having your period at a relatively young age. One reason women live longer than men may be because women have periods. With that regular blood loss, they are relatively iron deficient. Iron may play an important role in the production of oxygen free radicles that contribute to aging and age-related diseases and perhaps by having less iron, fewer of these damaging molecules are generated in our bodies. The least you should do is not take supplemental iron unless your health care provider gives you a good reason for doing so. The other thing to do is avoid red meat which is the most important source of iron in our diet.

If they answer age 40-55, give (0)

You stopped having your period at a bit younger than average age or at the average age women go through menopause. One reason women live longer than men may be because women have periods. With that regular blood loss, they are relatively iron deficient. Iron may play an important role in the production of oxygen free radicles that contribute to aging and age-related diseases and perhaps by having less iron, fewer of these damaging molecules are generated in our bodies. The least you should do is not take supplemental iron unless your health care provider gives you a good reason for doing so. The other thing to do is avoid red meat which is the most important source of iron in our diet.

If they answer age 56-59, give (+10)

You went through menopause at an older than average age for menopause (about 52 years). One reason women live longer than men may be because women have periods. With that regular blood loss, they are relatively iron deficient. Iron may play an important role in the production of oxygen free radicles that contribute to aging and age-related diseases and perhaps by having less iron, fewer of these damaging molecules are generated in our bodies. Thus, you were likely iron deficient for a longer period of time than most, which may be a big advantage and which might bode well for you ability to achieve exceptional longevity. In the mean time, you should not take supplemental iron unless your health care provider gives you a good reason for doing so. The other thing to do is avoid red meat which is the most important source of iron in our diet.

If they answer age 60 or greater, give (+15)

You went through menopause at a substantially older than average age for menopause (about 52 years). One reason women live longer than men may be because women have periods. With that regular blood loss, they are relatively iron deficient. Iron may play an important role in the production of oxygen free radicles that contribute to aging and age-related diseases and perhaps by having less iron, fewer of these damaging molecules are generated in our bodies. Thus, you were likely iron deficient for a longer period of time than most, which may be a big advantage and which might bode well for you ability to achieve exceptional longevity. In the mean time, you should not take supplemental iron unless your health care provider gives you a good reason for doing so. The other thing to do is avoid red meat which is the most important source of iron in our diet.

What is claimed is:

1. A computer-implemented method comprising:
(A) providing health-related questions to a user;
(B) receiving answers to the questions from the user;
(C) identifying an estimate of a life expectancy of the user based on the received answers without charging the user a fee; and
(D) providing advice to the user for increasing the life expectancy based on the received answers in exchange for a fee paid by the user.

2. The method of claim 1, wherein the advice includes information descriptive of factors related to increasing the life expectancy of the user and information descriptive of factors related to decreasing the life expectancy of the user.

3. The method of claim 1, wherein the fee is a one-time fee charged to the user in exchange for the advice.

4. The method of claim 1, wherein the fee is a recurring fee charged to the user over a period of time, and wherein the advice includes advice provided to the user over the period of time.

5. A computer-implemented method comprising:
(A) providing health-related questions to a user;
(B) receiving answers to the questions from the user;
(C) identifying an estimate of a life expectancy of the user based on the received answers;
(D) identifying an age of the user;
(E) identifying a gender of the user; and
(F) based on the received answers, the identified age, and the identified gender, providing advice to the user for increasing the life expectancy by following a plan of prevention to be carried out with a health-care provider of the user.

6. The method of claim 5, wherein (F) includes charging a fee to the user in exchange for providing the user with the advice.

7. The method of claim 5, wherein the advice includes information descriptive of factors related to increasing the life expectancy of the user and information descriptive of factors related to decreasing the life expectancy of the user.

8. A method comprising:
(A) charging an organization a fee to obtain access to a computer-implemented life expectancy system;
(B) providing the organization with access to the life expectancy system, wherein the life expectancy system:
(1) provides health-related questions to a member of the organization;
(2) receives answers to the questions from the member;
(3) identifies an estimate of a life expectancy of the member based on the received answers; and
(4) provides advice to the member for increasing the life expectancy based on the received answers;
wherein (B)(1)-(B)(4) are performed without charging an additional fee to the member.

9. A method comprising:
(A) charging an entity a fee to obtain access to a computer-implemented life expectancy system;
(B) providing the entity with access to the life expectancy system, wherein the life expectancy system:
(1) provides health-related questions to a person selected by the entity;
(2) receives answers to the questions from the person;
(3) identifies an estimate of a life expectancy of the person based on the received answers; and
(4) recommends advice to provide to the person for increasing the life expectancy based on the received answers.

10. The method of claim 9, wherein (A) comprises charging the entity the fee to provide the recommended advice to the person.
